(19) United States
(12) Patent Application Publication
(10) Pub. No.: US 2022/0054509 A1
(43) Pub. Date: Feb. 24, 2022

(54) METHODS OF TREATMENT FOR DISEASE FROM CORONAVIRUS EXPOSURE

(71) Applicant: Ensemble Group Holdings, Scottsdale, AZ (US)

(72) Inventors: Michael David KUO, Scottsdale, AZ (US); Clifford SCHLECHT, Lookout Mountain, GA (US)

(21) Appl. No.: 17/516,449

(22) Filed: Nov. 1, 2021

Related U.S. Application Data
(63) Continuation-in-part of application No. 17/194,931, filed on Mar. 8, 2021, now Pat. No. 11,160,814.
(60) Provisional application No. 63/063,149, filed on Aug. 7, 2020.
(60) Provisional application No. 63/021,544, filed on May 7, 2020.

(51) Int. Cl. A61K 31/58; A61K 9/00; A61K 31/522; A61K 31/404; A61K 31/47; A61K 31/381; A61K 31/192

(52) U.S. Cl. CPC A61K 31/58; A61K 9/0075; A61K 31/522; A61K 31/192; A61K 31/47; A61K 31/381; A61K 31/404

(57) ABSTRACT

Disclosed herein are methods of treating a disease in a subject which is the consequence of previous exposure of the subject to a virus, particularly SARS-CoV-2, the method comprising administration of an effective amount of one or more agents to the subject.

This application claims the benefit of the filing date as a continuation-in-part of the U.S. patent application Ser. No. 17/194,931, filed on Mar. 8, 2021, now allowed, which claims the benefit of priority of U.S. Provisional Patent Application Ser. No. 63/021,544 filed May 7, 2020, and also claims the benefit of priority of the U.S. Provisional Patent Application Ser. No. 63/063,149 filed Aug. 7, 2020, the disclosures of which are each incorporated by reference in their entireties for all purposes.

The worldwide COVID-19 pandemic rivals the 1918 influenza outbreak as the most significant health crisis in modern society. Although COVID-19, caused by the SARS-CoV-2 virus, has been observed to impact the heart, kidneys, and nervous and digestive systems, the disease predominantly impacts the respiratory system. Although the disease can remain undetected after infection for days or weeks before presenting symptoms, its effects on morbidity and mortality generally manifest quickly once the disease takes hold. Subjects will often present with gastrointestinal symptoms; however, fever, cough, and shortness of breath are the most common symptoms. Outcomes from infection span a wide range: an unknown fraction of subjects exposed to the virus do not present with any symptoms, some people experience mild to moderate symptoms akin to a cold or the flu, and a fraction of subjects experience a crisis that requires hospitalization, with oxygen treatment and often intubation often being necessary to overcome damage to the lungs.

Because the virus has only recently been encountered, very little is known about the effects of infection by the SARS-CoV-2 virus on survivors' long-term health. In the United Kingdom, up to 20% of patients reported persistent symptoms five weeks after COVID-19. This finding suggests that intervention with inhaled glucocorticoids might impact the rate of persistent long-term symptoms in COVID-19 (“long COVID”). Long-term pulmonary effects, blood disorders, and neurological damage are possible; however, both the newness of the disease and the lack of knowledge of the particular impact of the virus on the body and the survivor population's demographics make definitive predictions impossible.

Due to the widespread penetration of this disease and the concomitant number of survivors from all degrees of illness, many sequelae will likely emerge over the coming months and years. Understandably, attention has been mostly directed at compounds and methods for treating acute COVID-19. However, treating the long-term ailments derived from exposure to SARS-CoV-2 will increasingly be required.

### BRIEF DESCRIPTION

Provided herein is a method of treating a pulmonary disease which is the consequence of previous exposure of a subject to a severe acute respiratory syndrome coronavirus-2 (SARS-CoV-2), the method comprising administering to the subject of an effective amount of one or more corticosteroids.

The present disclosure further provides a method of treating dyspnea, shortness of breath, fatigue, cough, chest pain, and combinations thereof, which is a pulmonary sequela who had COVID-19, the method comprising administering to the subject in need thereof an effective amount of one or more corticosteroids.

In certain embodiments, the one or more corticosteroids are chosen from hydrocortisone, methylprednisolone, budesonide, beclomethasone, fluticasone, fluticasone furoate, fluticasone propionate, mometasone, mometasone furoate, and vamorolone, such as budesonide, any of which can be administered twice daily.

In certain embodiments, the one or more corticosteroids are administered immediately after exposure to SARS-CoV-2 before initial recovery. In certain embodiments, the one or more corticosteroids are administered immediately after exposure to SARS-CoV-2 after peak disease severity during recovery. In certain embodiments, the one or more corticosteroids are administered after viral recovery. In certain embodiments, the one or more corticosteroids are administered or maintained continuously. In certain embodiments, the one or more corticosteroids are administered when the subject expresses a symptom which is a consequence of the previous exposure of the SARS-CoV-2 or is about to partake in an activity that may precipitate the symptom. In certain embodiments, the one or more corticosteroids are administered in any combination of these aforementioned dosing schedules.

In certain embodiments, the method further comprises administering one or more leukotriene antagonists, such as zafirlukast, montelukast, and zileuton. In certain embodiments, the method further comprises administering one or more beta-agonists. In certain embodiments, the method further comprises administering one or more methylxanthines, such as theophylline, dyphylline, and aminophylline. In certain embodiments, the method further comprises administering one or more bronchodilators, for example, an anticholinergic or an antimuscarinic, such as tiotropium, ipratropium, aclidinium, glycopyrronium, and/or salts thereof. In certain embodiments, the one or more bronchodilators comprises a beta-2 agonist, such as salbutamol, albuterol, salmeterol, and formoterol.

In certain embodiments, the exposure of the subject to the SARS-CoV-2 virus is confirmed by a positive titer for the SARS-CoV-2, by a positive titer for antibodies to the SARS-CoV-2, or by one or more recognized clinical symptoms associated with infection by SARS-CoV-2. In certain embodiments, the one or more recognized clinical symptoms are chosen from cough, shortness of breath, difficulty breathing, asthma, fever, chills, repeated shaking, muscle pain, headache, sore throat, loss of taste, loss of smell, blood clotting, stroke, pernio, and chilblain (so-called “covid toes”).

The present disclosure provides a method of treating dyspnea, shortness of breath, fatigue, cough, chest pain, and combinations thereof, which is a pulmonary sequela who had COVID-19, the method comprising administering to the subject in need thereof an effective amount of budesonide and/or a salt thereof. In certain embodiments, the effective amount of budesonide is 0.64 mg per day administered via inhalation. In certain embodiments, the effective amount of budesonide 32 μg per nostril is administered nasally, for example, as needed every one to two hours.

The present disclosure provides a method of treating dyspnea, shortness of breath, fatigue, cough, chest pain, and combinations thereof, which is a pulmonary sequela who had COVID-19, the method comprising administering to the subject in need thereof an effective amount of fluticasone and/or a salt thereof. In certain embodiments, the effective amount of fluticasone is 200 μg per day administered via inhalation. In certain embodiments, the effective amount of fluticasone 50 μg per nostril administered nasally, for example, as needed every one to two hours.

The present disclosure also provides a method of reducing, delaying the onset of, or preventing symptoms which are the consequence of previous exposure of a subject to a severe acute respiratory syndrome coronavirus-2 (SARS-CoV-2), the method comprising administering to the subject of an effective amount of one or more corticosteroids.

### DETAILED DESCRIPTION

SARS-CoV-2 (hereafter referenced as “SARS2”, “COVID-19” or “COVID”) was first reported in December 2019 in Wuhan, China, with first published reports emerging in January of 2020. While it is a member of the coronavirus family and initially known to cause viral pneumonia, the art initially taught that it was generally similar to other pulmonary-tropic coronaviruses, except less contagious and less deadly than other recent coronavirus outbreaks, namely SARS-CoV-1 and MERS-CoV (hereafter referenced as “SARS1” and “MERS” respectively).

It is now becoming increasingly clear that COVID infections are very distinct from other coronavirus infections. As described herein, SARS-CoV-2's viral transmission kinetics, patterns, duration, and time to peak of viral shedding, R naught (abbreviated Ro), number of viral particles in different body compartments, and clinical natural history, among many other factors, are profoundly different from SARS and MERS, and other viruses in general, such as influenza, which SARS-CoV-2 was also initially compared to. Critically, although SARS1 and MERS are members of the same viral family as COVID, all infecting the lungs, any attempt to project acute, intermediate, and long-term consequences from SARS2 infection, as has been described herein, are complete guesswork. For example, the clinical and laboratory manifestations of SARS2 are far different from SARS1, MERS, and other viral pneumonia, with symptoms of headaches, loss of smell, multi-system inflammatory syndromes in children, myocarditis, pulmonary hypertension, thromboembolic disorders, stroke, renal failure, high serum levels of ferritin, LDH, and C-reactive protein, distinct serum immune signature as well as metabolite profiles, among a host of other phenomena that all clearly distinguish SARS2 from SARS2, MERS, and other viral pneumonia, and thus reinforcing the fact that forecasting of acute, intermediate and long term sequelae of the disease without any comparable precedent, complete guesswork at this time.

To date, almost all studies have focused on investigating and treating severe and hospitalized COVID-19 infection. However, there is currently little knowledge on therapeutic targets in early COVID-19 infection or during or after recovery to subsequently prevent or mitigate progression and clinical deterioration. Early reports describing COVID-19 infection from China, Italy, and the United States significantly underrepresented the number of patients with asthma and chronic obstructive pulmonary disease (COPD). In-vitro studies have shown that inhaled glucocorticoids reduce SARS-CoV-2 replication in airway epithelial cells and downregulated the expression of angiotensin-converting enzyme-2 (ACE2) and transmembrane protease serine 2 (TMPRSS2) genes, which are critical for viral cell entry.

Herein is disclosed that, contrary to the art, SARS2 has unique and unprecedented immediate recovery and convalescent-phase, intermediate phase, and long-term phase clinical sequelae upon previously SARS2 infected patients that affect the lungs both physically and structurally as well as functionally and physiologically. These effects can be mitigated with both specific treatments and formulations and delivery methods and the timing of their delivery and in which patient populations said such treatments are tailored for delivery to.

As disclosed herein, in a cohort of clinically recovered SARS2 patients, with follow-up from as little as 1-week post medically deemed clinical recovery to as far as 6 months out, greater than 15% of these patients presented evidence by medical imaging-chest x-ray and CT of long-lasting structural lung damage, including but not limited: to volume loss and fibrosis and scarring affecting the pulmonary parenchyma as well as small, medium and large airways, as well as the pleura, pulmonary interstitium and secondary lobules (including centrilobular and perilymphatic areas). Additionally, varying degrees of ground-glass opacities, traction on airways, distorted architectural interfaces, consolidation, and interstitial thickening was observed over time, and this was independent of clinical severity of the SARS2 active phase infection, length of hospital duration, and available treatments administered at that time (oxygen support—including ECMO, positive pressure air support, or mechanical ventilation, or medical/pharmacologic treatment including antiviral therapies or steroids or broad or targeted anti-inflammatory or immune-modulating agents).

Further, similar changes were observed in objective pulmonary function as measured by objective testing measures as described below, and clinical symptoms with patients demonstrating persistent and unresolved fatigue and airway reactivity that was both generalized as well as activity mediated (including exercise mediated or exertional) as well as hypersensitized to previously tolerated stimuli or antigens. Further, patients demonstrated persistent, slowly resolving, or progressively worsening dyspnea, shortness of breath, persistent or intermittent dry and wet or productive cough as well as drops in arterial blood gases, oxygen content, and saturation, oxygen diffusivity, or gas exchange.

As disclosed below, in a cohort of clinically recovered SARS2 patients, with follow-up from as little as 1-week post medically deemed clinical recovery to as far as 6 months out, greater than 30% of these patients had evidence of long-lasting functional lung damage or loss in pulmonary capacity or reserve which was observed over time and which was independent of clinical severity of the SARS2 active phase infection, length of hospital duration, and available treatments administered at that time (oxygen support—including ECMO, positive pressure air support, or mechanical ventilation, or medical/pharmacologic treatment including antiviral therapies or steroids or broad or targeted anti-inflammatory or immune-modulating agents). Testing measures to assess this included the methods described below.

As discussed by Gandhi, M., et al., Asymptomatic transmission, the Achilles' heel of current strategies to control COVID-19, 382 NEW ENG. J. MED., 2158-2160 (2020), a key factor in the transmissibility of COVID-19 is the high level of SARS-CoV-2 shedding in the upper respiratory tract (versus SARS-CoV1 and MERS-CoV, which are primarily lower respiratory tract tropic) even among presymptomatic patients, which distinguishes it from SARS-CoV-1, where replication occurs mainly in the lower respiratory tract. See

Wölfel, R., et al., Virological assessment of hospitalized patients with COVID-2019, 581 NATURE 465-469 (2020); and Cheng, P. K., et al., Viral shedding patterns of coronavirus in patients with probable severe acute respiratory syndrome, 363 LANCET 1699, 1699-1700 (2004). Viral loads with SARS-CoV-1, which are associated with symptom onset, peak a median of 5 days later than viral loads with SARS-CoV-2, which makes symptom-based detection of infection more effective in the case of SARS CoV-1. To, K. K. W., et al., Temporal profiles of viral load in posterior oropharyngeal saliva samples and serum antibody responses during infection by SARS-CoV-2: an observational cohort study, 20 LANCET INFECTIOUS DISEASES, P565-574 (2020), available at https://www.thelancet.com/journals/laninf/article/PIIS1473-3099(20)30196-1/fulltext. With influenza, persons with the asymptomatic disease generally have lower quantitative viral loads in secretions from the upper respiratory tract than from the lower respiratory tract and a shorter duration of viral shedding than persons with symptoms which decreases the risk of transmission from paucisymptomatic persons (i.e., those with few symptoms).

Ip, D. K., et al., Viral shedding and transmission potential of asymptomatic and paucisymptomatic influenza virus infections in the community, 64 CLINICAL INFECTIOUS DISEASES, 736-742 (2017) reported that more than half the residents of this skilled nursing facility (27 of 48) who had positive tests were asymptomatic at testing. Moreover, live coronavirus sheds at high concentrations from the nasal cavity even before symptom development.

Unlike SARS1 and MERS, COVID has a large proportion of completely asymptomatic patients prone to a high transmission before developing symptoms, viral loads peak earlier and taper slower and later, has a much smaller proportion of patients who require treatment or hospitalization, and a far lower fatality rate. Below are additional data that highlight characteristics of SAR2.

### SARS2 Variants

The SARS2 virus continues to mutate and present new variants in the human population. Examples of clinically concerning strains include B1.526, also called the New York variant, which includes LSF, T95I, D253G, E484K, S477N, D614G, and A701V among other mutations, B.1.427/B.1.429/20C/L452R, also called CAL.20, in California, B.1.1.7 in the United Kingdom (UK), B.1.351 in South Africa, and P.1 in Brazil. See SARS-CoV-2 Variants, US CENTERS FOR DISEASE CONTROL AND PREVENTION, https://www.cdc.gov/coronavirus/2019-ncov/cases-updates/variant-surveillance/variant-info.html (last updated Jan. 31, 2021). At least four large databases have sequenced the SARS-CoV-2 virus and listed variants by time, clade, country, etc. See GISAID, https://www.gisaid.org (last visited Mar. 4, 2021); Covid-Miner, https://covid-miner.ifo.gov.it/app/variants (last visited Mar. 4, 2021); Genomic epidemiology of novel coronavirus—Global subsampling, https://nextstrain.org/ncov/global (last visited Mar. 4, 2021); and Global.health, https://global.health (last visited Mar. 4, 2021).

In the UK, a variant of SARS-CoV-2 known as B.1.1.7 or the “alpha variant” emerged. This variant carries many mutations and has since been detected around the world, including in the United States (US). This variant was first detected in the US at the end of December 2020. In January 2021, scientists from the UK reported early evidence that suggests the B.1.1.7 variant may be associated with an increased risk of death compared with other variants.

In South Africa, another variant of SARS-CoV-2 known as B.1.351 or the “beta variant” emerged independently of B.1.1.7. According to a non-peer-reviewed preprint article, this variant shares some mutations with B.1.1.7. Cases attributed to B.1.351 have been detected outside of South Africa, and this variant was first detected in the US at the end of January 2021. Preliminary evidence from non-peer-reviewed publications suggests that the Moderna mRNA-1273 vaccine currently used in the US may be less effective against this variant.

In Brazil, a variant of SARS-CoV-2 known as P.1 or the “gamma variant” emerged; it was first identified in November 2020 in travelers from Brazil who arrived in Japan and designated in January 2021. This variant was detected in the US at the end of January 2021. The P.1 variant has 17 unique mutations, including three in the receptor-binding domain of the spike protein (K417T, E484K, and N501Y), according to non-peer-reviewed preprint articles. There is evidence to suggest that some of the mutations in the P.1 variant may affect the ability of antibodies (from natural infection or vaccination) to recognize and neutralize the virus.

In India, a variant of SARS-CoV-2 known as B.1.617.2 or the “delta variant” emerged in India. It was first identified in October 2020 and designated in May 2021. Reinfections happened, with smaller occurrence rate than vaccinated infections. Vaccination efficacy is reduced for non-severe disease. The delta variant has mutation at L452R, T478K, and P681R.

In certain embodiments, the subject was previously exposed to the B1.526 variant of SARS-CoV-2. In certain embodiments, the subject was previously exposed to the B.1.427/B.1.429/20C/L452R (CAL.20) variant of SARS-CoV-2. In certain embodiments, the subject was previously exposed to the B.1.1.7 variant of SARS-CoV-2. In certain embodiments, the subject was previously exposed to the B.1.351 variant of SARS-CoV-2. In certain embodiments, the subject was previously exposed to the P.1 variant of SARS-CoV-2.

In certain embodiments, the subject was previously exposed to the delta variant of SARS-CoV-2.

Concerning mutations in the B.1.17 variant are the 69/70 deletion, 144Y deletion, N501Y, A570D, D614G, and P681H. Concerning mutations in the P.1 variant are E484K, K417N/T, N501Y, and D614G. Concerning mutations in the B.1.351 variant are K417N, E484K, N501Y, and D614G.

One specific mutation, called D614G, is shared by these three variants. It gives the variants the ability to spread more quickly than the predominant viruses. There also is epidemiologic evidence that variants with this specific mutation spread more quickly than viruses without the mutation. This mutation was one of the first documented in the US in the pandemic's initial stages, after having first circulated in Europe. See McCarthy et al., Recurrent deletions in the SARS-CoV-2 spike glycoprotein drive antibody escape, SCIENCE 10.1126/science.abf6950 (2021), available at https://science.sciencemag.org/content/early/2021/02/02/science.abf6950.

In certain embodiments, the subject was previously exposed to a variant of SARS-CoV-2 having one or more mutations chosen from 69/70 deletion, 144Y deletion, N501Y, A570D, D614G, P681H, E484K, K417N/T, K417N, and D614G. In certain embodiments, the subject was previously exposed to a variant of SARS-CoV-2 having one or more mutations chosen from N501Y, E484K, K417N/T, and D614G. In certain embodiments, the subject was previously exposed to a variant of SARS-CoV-2 having a D614G mutation.

### Exemplary Embodiments

Provided herein are the following specific embodiments:

Embodiment 1. A method of treating a pulmonary disease which is the consequence of previous exposure of a subject to a severe acute respiratory syndrome coronavirus-2 (SARS-CoV-2), the method comprising administration to the subject of an effective amount of one or more agents chosen from antiasthmatics, bronchodilators, and tyrosine kinase inhibitors.

Embodiment 2. The method as recited in Embodiment 1, wherein the one or more agents comprises one or more antiasthmatics.

Embodiment 3. The method as recited in Embodiment 2, wherein the one or more antiasthmatics comprises a corticosteroid.

Embodiment 4. The method as recited in Embodiment 3, wherein the corticosteroid is chosen from hydrocortisone, methylprednisolone, budesonide (PULMICORT®, RHINOCORT®, ENTOCORT®), beclomethasone (QVAR®), fluticasone, fluticasone furoate (ARNUITY® ELLIPTA®), fluticasone propionate (FLOVENT®, FLONASE®, FLIXOTIDE), mometasone, mometasone furoate (NASONEX®, ASMANEX®, ELOCON®), and vamorolone.

Embodiment 5. The method as recited in any one of Embodiments 2-4, wherein the one or more antiasthmatics comprises a leukotriene antagonist.

Embodiment 6. The method as recited in Embodiment 5, wherein the leukotriene antagonist is chosen from zafirlukast (ACCOLATE®), montelukast (SINGULAIR®), and zileuton (ZYFLO®).

Embodiment 7. The method as recited in any one of Embodiments 2-6, wherein the one or more antiasthmatics comprises a beta agonist.

Embodiment 8. The method as recited in any one of Embodiments 2-7, wherein the one or more antiasthmatics comprises a methylxanthine.

Embodiment 9. The method as recited in Embodiment 8, wherein the methylxanthine is chosen from theophylline, dyphylline, and aminophylline.

Embodiment 10. The method as recited in any one of Embodiments 1-9, wherein the one or more agents comprises one or more bronchodilators.

Embodiment 11. The method as recited in Embodiment 10, wherein the one or more bronchodilators comprises an anticholinergic.

Embodiment 12. The method as recited in either one of Embodiments 10 and 11, wherein the one or more bronchodilators comprises an antimuscarinic.

Embodiment 13. The method as recited in Embodiment 12, wherein the antimuscarinic is chosen from tiotropium (SPIRIVA®), ipratropium (ATROVENT®), aclidinium (BRETARIS® GENUAIR, EKLIRA GENUAIR, TUDORZA® PRESSAIR®), and glycopyrronium (ROBINUL, CUVPOSA®, SEEBRI®).

Embodiment 14. The method as recited in any one of Embodiments 10-13, wherein the one or more bronchodilators comprises a beta-2 agonist.

Embodiment 15. The method as recited in Embodiment 14, wherein the beta-2 agonist is chosen from salbutamol (albuterol, VENTOLIN®) and salmeterol (SEREVENT®, AEROMAX).

Embodiment 16. The method as recited in Embodiment 1, wherein the one or more agents comprises a tyrosine kinase inhibitor.

Embodiment 17. The method as recited in Embodiment 16, wherein the tyrosine kinase inhibitor has inhibitory activity towards one or more receptors chosen from platelet-derived growth factor (PDGF), fibroblast growth factor receptor 1 (FGFR1), fibroblast growth factor receptor 2 (FGFR2), fibroblast growth factor receptor 3 (FGFR3), vascular endothelial growth factor receptor 1 (VEGFR1), vascular endothelial growth factor receptor 2 (VEGFR2), and vascular endothelial growth factor receptor 3 (VEGFR3).

Embodiment 18. The method as recited in Embodiment 16, wherein the tyrosine kinase inhibitor is nintedanib (OFEV®).

Embodiment 19. The method as recited in Embodiment 16, wherein the tyrosine kinase inhibitor is pirfenidone (ESBRIET®, PIRESPA®, ETUARY®).

Embodiment 20. A method of reducing pulmonary symptoms which are the consequence of previous exposure of a subject to a severe acute respiratory syndrome coronavirus-2 (SARS-CoV-2), the method comprising administration to the subject of an effective amount of one or more agents chosen from antiasthmatics, bronchodilators, and tyrosine kinase inhibitors.

Embodiment 21. A method of delaying the onset of pulmonary symptoms which are the consequence of previous exposure of a subject to a severe acute respiratory syndrome coronavirus-2 (SARS-CoV-2), the method comprising administration to the subject of an effective amount of one or more agents chosen from antiasthmatics, bronchodilators, and tyrosine kinase inhibitors.

Embodiment 22. A method of preventing pulmonary symptoms which are the consequence of previous exposure of a subject to a severe acute respiratory syndrome coronavirus-2 (SARS-CoV-2), the method comprising administration to the subject of an effective amount of one or more agents chosen from antiasthmatics, bronchodilators, and tyrosine kinase inhibitors.

Embodiment 23. The method as recited in any one of Embodiments 20-21, wherein the symptoms are chosen from cough, shortness of breath, difficulty breathing, asthma, fever, chills, repeated shaking, muscle pain, headache, sore throat, loss of taste, loss of smell, bloodclotting, stroke, pernio, and chilblain.

Embodiment 24. The method as recited in any one of Embodiments 1-23, wherein the exposure of the subject to the SARS-CoV-2 virus is confirmed by a positive titer for the SARS-CoV-2.

Embodiment 25. The method as recited in any one of Embodiments 1-23, wherein the exposure of the subject to the SARS-CoV-2 virus is confirmed by a positive titer for antibodies to the SARS-CoV-2.

Embodiment 26. The method as recited in any one of Embodiments 1-23, wherein the exposure of the subject to the SARS-CoV-2 virus is confirmed by one or more recognized clinical symptoms associated with infection by SARS-CoV-2.

### Agents

In certain embodiments, the one or more agents comprise one or more antiasthmatics. In certain embodiments, the one or more antiasthmatics comprises a corticosteroid. In certain embodiments, the corticosteroid is chosen from hydrocortisone, methylprednisolone, budesonide (Pulmicort®, Rhinocort®, Entocort®), beclomethasone (Qvar®), fluticasone, fluticasone furoate (Arnuity® Ellipta®), fluticasone propionate (Flovent®, Flonase®, Flixotide), mometasone, mometasone furoate (Nasonex®, Asmanex®, Elocon®), and vamorolone.

Budesonide is a potent corticosteroid available for intranasal, inhaled, rectal, and oral administration. The nasal form has been used for allergic rhinitis, the respiratory inhalation used for asthma or COPD, rectal form to treat ulcerative colitis, and orally to treat Crohn's disease and ulcerative colitis.

For patients 12 years or older, the US Food and Drug Administration (FDA) has recommended budesonide at 2 sprays (32 μg per spray) in each nostril once daily. After clinical response has been obtained, decrease to 1 spray in each nostril once daily. If no response after 2 weeks, consult a health care provider.

For children 6 to 11 years, the recommended initial dose is 1 spray (32 μg/spray) in each nostril once daily. If symptoms do not improve, the dose may be increased to 2 sprays in each nostril once daily. After clinical response has been obtained, decrease to 1 spray in each nostril once daily. Non-prescription use in younger children is intended to be assisted by an adult. If the child uses this product for longer than 2 months per year, or, if there is no response to treatment after 2 weeks, consult a pediatrician.

If the patient was dosed previously on bronchodilators alone, give 0.5 mg once daily or 0.25 mg twice daily via jet nebulizer. In symptomatic pediatric patients not responding to non-steroidal therapy, a starting dose of 0.25 mg once daily may be considered. The max for these patients is 0.5 mg/day. If the patient was previously on inhaled corticosteroids, give 0.5 mg once daily or 0.25 mg twice daily; one may give 0.5 mg twice daily via jet nebulizer. The max for these patients is 1 mg/day. If the patient was previously taking oral corticosteroids, give 0.5 mg twice daily or 1 mg once daily via jet nebulizer. The max for these patients is 1 mg/day.

If once-daily treatment does not provide adequate control, the total daily dose can be increased and/or administered as a divided dose. In these embodiments, titrate to the lowest effective dose once sequalae stability is achieved. The National Asthma Education and Prevention Program (NAEPP) Expert Panel defines low dose therapy as 0.5 mg/day, medium dose therapy as 1 mg/day, and high dose therapy as 2 mg/day for children 5 to 11 years. The NAEPP defines low dose therapy as 0.25 to 0.5 mg/day, medium dose as 0.5 to 1 mg/day, and high dose therapy as more than 1 mg/day for children 1 to 4 years. Short-term, high-dose nebulized budesonide can have an early clinical effect by day 2 of treatment.

For adults using the oral inhalation dosage (Pulmicort Flexhaler inhalation powder) the recommended starting dosage is 360 μg inhaled orally twice daily. In some adult patients, a starting dose of 180 μg twice daily may be adequate. The maximum dose in these patients is 720 μg inhaled twice daily. Titrate to the lowest effective dose once sequalae stability is achieved.

For children and adolescents 6 years and older, the recommended starting dosage is 180 μg inhaled orally twice daily. In some patients, a starting dosage of 360 μg twice daily may be appropriate. The maximum dose in these patients is 360 μg inhaled twice daily. Titrate to the lowest effective dose once sequalae stability is achieved. The NAEPP Expert Panel defines low dose therapy for budesonide dry powder inhalers as 180 to 400 μg/day, medium dose therapy as greater than 400 to 800 μg/day, and high dose therapy as greater than 800 μg/day for children ages 5 to 11 years.

For children 5 years and younger, the recommended starting dosage is 180 μg inhaled orally twice daily. In some patients, a starting dosage of 360 μg twice daily may be appropriate. The maximum dose in these patients is 360 μg inhaled twice daily. Titrate to the lowest effective dose once asthma stability is achieved. The NAEPP Expert Panel allows for dry powder inhalation use in children as young as 5 years of age.

Fluticasone is a highly selective agonist at the glucocorticoid receptor with negligible activity at androgen, estrogen, or mineralocorticoid receptors, thereby producing anti-inflammatory and vasoconstriction effects. In certain embodiments, fluticasone is a salt, such as fluticasone furoate (Arnuity® Ellipta®) or fluticasone propionate (Flovent®, Flonase®, Flixotide).

The recommended starting dosage of inhaled fluticasone for patients aged 4 years and older who are not on an inhaled corticosteroid is 88 μg (2 inhalations of 44 μg fluticasone propionate) twice daily by oral inhalation, about 12 hours apart. The maximum dosage for these patients is 880 μg twice daily. The starting dosage is based on previous COVID therapy and sequalae severity. If symptoms arise between doses, an inhaled short-acting beta2-agonist should be used for immediate relief.

Individual patients can experience a variable time to onset and degree of symptom relief. Maximum benefit may not be achieved for 1 to 2 weeks or longer after starting treatment. For other patients, and for patients who do not respond adequately to the starting dosage after 2 weeks of therapy, higher dosages may provide additional sequalae control.

If a dosage regimen fails to provide adequate control of the sequalae, the therapeutic regimen should be re-evaluated and additional therapeutic options, e.g., replacing the current strength with a higher strength, initiating an inhaled corticosteroid and long-acting beta2-agonist (LABA) combination product, or initiating oral corticosteroids, should be considered. After sequalae stability has been achieved, one can titrate to the lowest effective dosage to reduce the possibility of side effects.

Fluticasone is available as an inhalation aerosol, an inhaler containing a pressurized metered-dose aerosol canister containing 120 metered inhalations, and fitted with a counter. The doses are 44 μg, 110 μg, and 220 μg fluticasone propionate from the mouthpiece per actuation.

For nasal administration, the recommended dose of fluticasone in week 1 is two sprays in each nostril once daily (50 μg). Week 2 through 6 months—use 1 or 2 sprays in each nostril once daily, as needed to treat symptoms. In certain embodiments, fluticasone is administered one spray (50 μg) per nostril as needed to treat sequalae, such as cough.

Hydrocortisone is the name for the hormone cortisol when supplied as a medication. The initial dosage of hydrocortisone may vary from 20 mg to 240 mg hydrocortisone per day, depending on the severity of the sequalae.

Methylprednisolone (Depo-Medrol, Medrol, Solu-Medrol) is a synthetic glucocorticoid, primarily prescribed for its anti-inflammatory and immunosuppressive effects. Chemically, methylprednisolone is a synthetic pregnane steroid hormone derived from hydrocortisone and prednisolone. Depending on the sequelae being treated, the initial methylprednisolone dosage may vary from 4 mg to 48 mg per day.

Mometasone (Nasonex®, Asmanex®, Elocon®), also known as mometasone furoate, is a steroid medication used to treat certain skin conditions, hay fever, and asthma. Specifically, it is used to prevent rather than treat asthma attacks. It can be applied to the skin, inhaled, or used in the nose. For intranasal treatment in adults and adolescents 12 years and older, the recommended dose is two sprays (50 μg per spray) in each nostril once daily, for a total daily dose of 200 μg. The recommended dose for children of 2 to 11 years is one spray (50 μg per spray) in each nostril once daily, for a total daily dose of 100 μg.

Vamorolone is being investigated as a first-in-class dissociative steroid with a lower incidence of corticosteroid-associated adverse effects. The pivotal VISION-DMD study met its primary endpoint of superiority in the change of time to stand from supine positioning to standing (TTSTAND) velocity with vamorolone 6 mg/kg/day versus placebo (p=0.002) with a treatment difference of 0.06 [95% CI: 0.02-0.10] rises/second from baseline at 24 weeks. The study also demonstrated the superiority of both vamorolone dose levels (2 and 6 mg/kg/day) versus placebo across multiple secondary endpoints. Importantly, vamorolone showed a favorable safety and tolerability profile compared to prednisone. Vamorolone did not stunt growth, as validated in the current 24-week study, in which vamorolone 6 mg/kg/day versus prednisone 0.75 mg/kg/day showed a significant difference in growth velocity (p=0.02). Furthermore, statistically significant differences between vamorolone (2 and 6 mg/kg/day) and prednisone groups were seen at week 24 in biomarkers assessing bone health: osteocalcin, Procollagen 1 N-Terminal Propeptide (P1NP), and Type I Collagen C-Telopeptides (CTX) (p<0.001 for vamorolone both doses vs. prednisone for all three parameters). Based on the available data, vamorolone could emerge as a promising alternative to existing corticosteroids.

Lower doses generally suffice in situations of less severity, while in selected patients, higher initial doses may be required. The initial dosage should be maintained or adjusted until a satisfactory response is noted. If the clinical response is not satisfactory after a reasonable time, one or more corticosteroids may be discontinued and the patient transferred to other appropriate therapy. Dosages are variable and must be individualized based on the disease under treatment and the patient's response. After a favorable response is noted, the proper maintenance dosage should be determined by decreasing the initial drug dosage in small decrements at appropriate time intervals until the lowest dosage, which will maintain an adequate clinical response is reached.

Constant monitoring is needed for drug dosage, including changes in clinical status secondary to remissions or exacerbations in the disease process, the patient's drug responsiveness, and the effect of patient exposure to stressful situations not directly related to the disease entity under treatment. In this latter situation, the dosage of one or more corticosteroids may need to be adjusted for a time consistent with the patient's condition. If, after long-term therapy, the drug is to be stopped, it is recommended that it be withdrawn gradually rather than abruptly.

In certain embodiments, post-Covid airway inflammation and/or airflow obstruction is treated in in patients 12 years of age and older.

In certain embodiments, the patient is treated with Symbicort Dry powder inhaler (Turbohaler) or with MDI with a spacer (Rapihaler).

In certain embodiments, the method further comprises administering 3.5-4 μg formoterol. In certain embodiments, the method further comprises administering 3.5 μg formoterol. In certain embodiments, the method further comprises administering 3.75 μg formoterol. In certain embodiments, the method further comprises administering 4 μg formoterol. In certain embodiments, after the patient attains a stable dosage, the formoterol is down titrated.

In certain embodiments, two puffs of 140-180 μg budesonide are administered twice daily. In certain embodiments, two puffs of 140 μg budesonide are administered twice daily. In certain embodiments, two puffs of 150 μg budesonide are administered twice daily. In certain embodiments, two puffs of 160 μg budesonide are administered twice daily. In certain embodiments, two puffs of 175 μg budesonide are administered twice daily. In certain embodiments, two puffs of 180 μg budesonide are administered twice daily.

In certain embodiments, the one or more corticosteroids are administered as a dry powder via MDI spacer (tugboater).

In certain embodiments, one or two puffs of a composition comprising 150 μg or 175 μg budesonide and 3.5 μg or 4 μg or 5 μg formoterol is administered twice daily.

In certain embodiments, one puff of a composition comprising 150 μg budesonide and 3.5 μg formoterol is administered twice daily.

In certain embodiments, one puff of a composition comprising 150 μg budesonide and 4 μg formoterol is administered twice daily.

In certain embodiments, one puff of a composition comprising 150 μg budesonide and 5 μg formoterol is administered twice daily.

In certain embodiments, one puff of a composition comprising 175 μg budesonide and 3.5 μg formoterol is administered twice daily.

In certain embodiments, one puff of a composition comprising 175 μg budesonide and 4 μg formoterol is administered twice daily.

In certain embodiments, one puff of a composition comprising 175 μg budesonide and 5 μg formoterol is administered twice daily.

In certain embodiments, two puffs of a composition comprising 150 μg budesonide and 3.5 μg formoterol is administered twice daily.

In certain embodiments, two puffs of a composition comprising 150 μg budesonide and 4 μg formoterol is administered twice daily.

In certain embodiments, two puffs of a composition comprising 150 μg budesonide and 5 μg formoterol is administered twice daily.

In certain embodiments, two puffs of a composition comprising 175 μg budesonide and 3.5 μg formoterol is administered twice daily.

In certain embodiments, two puffs of a composition comprising 175 μg budesonide and 4 μg formoterol is administered twice daily.

In certain embodiments, two puffs of a composition comprising 175 μg budesonide and 5 μg formoterol is administered twice daily.

In certain embodiments, the one or more antiasthmatics comprises a leukotriene antagonist. In certain embodiments, the leukotriene antagonist is chosen from zafirlukast (Accolate®), montelukast (Singular®), and zileuton (Zyflo®). In certain embodiments, the one or more antiasthmatics comprises a beta-agonist, such as a long-acting beta-agonist. In certain embodiments, the one or more antiasthmatics comprise a methylxanthine. In certain embodiments, the methylxanthine is chosen from theophylline, dyphylline, and aminophylline.

In certain embodiments, the one or more agents comprises one or more bronchodilators. In certain embodiments, the one or more bronchodilators comprises an anticholinergic. In certain embodiments, the one or more bronchodilators comprises an antimuscarinic. In certain embodiments, the muscarinic is chosen from tiotropium (Spiriva®), ipratropium (Atrovent®), aclidinium (Bretaris® Genuair, Eklira Genuair, Tudorza® Pressair®), and glycopyrronium (Robinul, Cuvposa®, Seebri®). In certain embodiments, the one or more bronchodilators comprises a beta-2 agonist. In certain embodiments, the beta-2 agonist is chosen from salbutamol (albuterol, Ventolin®) and salmeterol (Serevent®, Aeromax).

In certain embodiments, the one or more agents comprise a tyrosine kinase inhibitor. In certain embodiments, the tyrosine kinase inhibitor has inhibitory activity towards one or more receptors chosen from platelet-derived growth factor (PDGF), fibroblast growth factor receptor 1 (FGFR1), fibroblast growth factor receptor 2 (FGFR2), fibroblast growth factor receptor 3 (FGFR3), vascular endothelial growth factor receptor 1 (VEGFR1), vascular endothelial growth factor receptor 2 (VEGFR2), and vascular endothelial growth factor receptor 3 (VEGFR3). In certain embodiments, the tyrosine kinase inhibitor is Nintedanib (OFEV®). In certain embodiments, the tyrosine kinase inhibitor is pirfenidone (ESBRIET®, PIRESPA®, ETUARY®).

In certain embodiments, the medicament is a corticosteroid. In certain embodiments, the corticosteroid is chosen from beclomethasone, budesonide, fluticasone, and mometasone.

In certain embodiments, the corticosteroid is administered alone. In certain embodiments, the corticosteroid is administered in combination with any other number of corticosteroids at any of the doses disclosed and can be administered up to 8 times per day. Any of the aforementioned Beta2 adrenoreceptor agonists (including the aforementioned LABAs and ultraLABAs) can be administered alone or in combination with any other number of Beta2 adrenoreceptor agonists at any of the doses disclosed and can be administered up to 8 times per day. Any of the aforementioned corticosteroids, beta2 adrenoreceptor agonists, and tyrosine kinase antagonists listed can be administered or delivered in any combination at any of the doses disclosed. They can be administered up to 8 times per day.

Without wishing to be bound by theory, post-acute SARS-CoV2 syndrome and long COVID are most visibly diseases of small airways. Beta2 agonists do not seem to affect the disease much, at least from a physiological perspective. So, the Beta2 agonist is not reaching the receptors, long COVID is mostly inflammatory, or both.

Median aerodynamic diameter (MAD) is one of two parameters influencing the deposition of inhaled particles, the other being the geometric standard deviation of the particle size distribution. The MAD is the value of aerodynamic diameter for which 50% of some quantity in a given aerosol is associated with particles smaller than the MAD, and 50% of the quantity is associated with particles larger than the MAD. It simplifies the true distribution of aerodynamic diameters of a given aerosol as a single value. It is used to describe those particle sizes for which deposition depends chiefly on inertial impaction and sedimentation. Mass median aerodynamic diameter (MMAD) is the MAD for mass. In certain embodiments, the particle size of the drug is extra fine (e.g., less than 2.1 μg MMAD), thus allowing it to distribute into peripheral small airways disease. The small particle size is often attained via the delivery device.

In certain embodiments, the device is chosen from metered-dose inhalers (MDI) with and without a spacer, dry power inhalers (DPI), and nebulizers. In certain embodiments, the device is chosen from Turbohaler, evohaler, Respimat, Adaptive aerosol delivery (Phillips Respironics), AKITA (Activaero), digihaler (Teva).

Albuterol (Accuneb, Proair HFA, Proair Respiclick, Proventil HFA, Ventolin HFA) is a SABA. It comes as a nebulizer solution and inhalers you use for fast symptom relief.

Beclomethasone (Beclovent, QVAR) is an inhaled corticosteroid. The usual dose is twice daily for long-term control.

Budesonide/formoterol (Symbicort) is a combination of an inhaled corticosteroid and a LABA. It comes as an inhaler for twice-daily use.

Breztri aerosphere (budesonide 160 μg, glycopyrrolate 9 μg, formoterol fumarate 4.8 μg). The formoterol component was modified from 4.5 μg in Symbicort to 4.8 μg in the Breztri formulation.

Fluticasone/salmeterol (Advair Diskus, Advair HFA) combines an inhaled corticosteroid and a LABA. This inhaler is used once a day.

Fluticasone/vilanterol (Breo Ellipta) combines an inhaled corticosteroid and a LABA. You use this inhaler once a day.

Ipratropium (Atrovent, Atrovent HFA) is a short-acting anticholinergic. It comes as a nebulizer solution and an inhaler. The dose is usually four times a day.

Ipratropium/Albuterol (Combivent Respimat, Duoneb) combines two bronchodilators in the form of a short-acting anticholinergic and a SABA. The usual dose for both the nebulizer solution and the inhaler is four times a day.

Tiotropium (Spiriva HandiHaler) is a LAMA. It is an inhaler you use once daily. Tiotropium is also available in combination with olodaterol, a LAB A, under the brand name, Stiolto.

GSK's Trelegy Ellipta is the first FDA-cleared drug approved for asthma and COPD. This triple therapy combines a steroid with a Beta2 agonist and an anti-muscarinic agent—fluticasone furoate 100 μg, vilanterol trifenatate 25 μg, and umeclidinium bromide 62.5 μg.

In certain embodiments, the medicament is beclomethasone. In certain embodiments, the dosage of beclomethasone is delivered by any one of the following methods: aerosol, nebulized, powder, metered, and inhalation. In certain embodiments, the dosage of beclomethasone is delivered via inhalation. In certain embodiments, the dosage of beclomethasone is any one of 20, 30, 40, 50, 60, 70, 80, 90, 100, 110, 120, 130, 140, 150, 160, 170, 180, 190, 200 mg. In certain embodiments, the dosage of beclomethasone is 40 mg. In certain embodiments, the dosage of beclomethasone is 80 mg.

In certain embodiments, the medicament is budesonide. In certain embodiments, the dosage of budesonide is delivered by any one of the following methods: aerosol, nebulized, powder, metered, and inhalation. In certain embodiments, the dosage of budesonide is delivered via inhalation. In certain embodiments, the dosage of budesonide is any one of 0.040, 0.045, 0.050, 0.055, 0.060, 0.065, 0.070, 0.075, 0.080, 0.085, 0.090, 0.095, 0.100, 0.105, 0.110, 0.115, 0.120, 0.125, 0.130, 0.135, 0.140, 0.145, 0.150, 0.155, 0.160, 0.165, 0.170, 0.175, 0.180, 0.185, 0.190, 0.195, 0.200, 0.205, 0.210, 0.215, 0.220, 0.225, 0.230, 0.235, 0.240, 0.245, 0.250, 0.255, 0.260, 0.265, 0.270, 0.275, 0.280, 0.285, 0.290, 0.295, 0.300, 0.305, 0.310, 0.315, 0.320, 0.325, 0.330, 0.335, 0.340, 0.345, 0.350, 0.355, 0.360, 0.365, 0.370, 0.375, 0.380, 0.385, 0.390, 0.395, 0.400, 0.405, 0.410, 0.415, 0.420, 0.425, 0.430, 0.435, 0.440, 0.445, 0.450, 0.455, 0.460, 0.465, 0.470, 0.475, 0.480, 0.485, 0.490, 0.495, and 0.500 mg. In certain embodiments, the dosage of budesonide is 0.080 mg. In certain embodiments, the dosage of budesonide is 0.160 mg. In certain embodiments, the dosage of budesonide is 0.320 mg.

In certain embodiments, the medicament is fluticasone propionate. In certain embodiments, the dosage of fluticasone propionate is delivered by any one of the following methods: aerosol, nebulized, powder, metered, and inhalation. In certain further embodiments, the dosage is delivered via inhalation. In yet further embodiments, the inhaled dosage is any one of 0.1, 0.15, 0.175, 0.2, 0.225, 0.25, 0.275, 0.3, 0.325, 0.35, 0.375, 0.4, 0.425, 0.45, 0.475, 0.5. 0.525, 0.55, 0.575, 0.6, 0.625, 0.65, 0.675, 0.7, 0.725, 0.75. 0.775, or 0.8 mg.

In certain embodiments, the medicament is mometasone. In certain embodiments, the dosage of mometasone is delivered by any one of the following methods: aerosol, nebulized, powder, metered, and inhalation. In certain embodiments, the dosage of mometasone is delivered via inhalation. In certain embodiments, the dosage of mometasone is any one of 50, 60, 70, 80, 90, 100, 110, 120, 130, 140, and 150 mg. In certain embodiments, the dosage of mometasone is 100 mg.

In certain embodiments, the medicament is fluticasone furoate.

In certain embodiments, the medicament is ciclesonide. In certain embodiments, the dosage of ciclesonide is delivered by any one of the following methods: aerosol, nebulized, powder, metered, and inhalation. In certain embodiments, the dosage of ciclesonide is delivered via inhalation. In certain embodiments, the dosage of ciclesonide is any one of 40, 50, 60, 70, 80, 90, 100, 110, 120, 130, 140, 150, 160, 170, 180, 190, 200, 210, 220, 230, 240, 250, 260, 270, 280, 290, 300, 310, and 320 mg. In certain embodiments, the dosage of ciclesonide is 80 mg. In certain embodiments, the dosage of ciclesonide is 160 mg.

In certain embodiments, the medicament is flunisolide. In certain embodiments, the dosage of flunisolide is delivered by any one of the following methods: aerosol, nebulized, powder, metered, and inhalation. In certain embodiments, the dosage of flunisolide is delivered via inhalation. In certain embodiments, the dosage of flunisolide is any one of 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, and 500 mg. In certain embodiments, the dosage of flunisolide is 250 mg.

In certain embodiments, the medicament comprises a combined dosage of mometasone and formoterol fumarate. In certain embodiments, the combined dosage of mometasone and formoterol fumarate is delivered by any one of the following methods: aerosol, nebulized, powder, metered, and inhalation. In certain embodiments, the combined dosage of mometasone and formoterol fumarate is delivered via inhalation. In certain embodiments, the combined dosage of mometasone and formoterol fumarate contains any one of 50, 60, 70, 80, 90, 100, 110, 120, 130, 140, and 150 mg of mometasone. In certain embodiments, the combined dosage of mometasone and formoterol fumarate contains any one of 200, 250, 300, 350, 400, 450, 500, 550, 600, 650, 700, 750 mg of formoterol fumarate.

In certain embodiments, the medicament is a beta2 adrenoreceptor agonist/activator. In certain embodiments, the beta2 adrenoreceptor agonist/activator is chosen from salmeterol, salmeterol xinafoate, formoterol, formoterol fumarate, arformoterol, bambuterol, clenbuterol, protokylol, and albuterol.

In certain embodiments, the medicament is salmeterol xinafoate. In certain embodiments, the dosage of salmeterol xinafoate is delivered by any one of the following methods: aerosol, nebulized, powder, metered, and inhalation. In certain embodiments, the dosage of salmeterol xinafoate is delivered via inhalation. In certain embodiments, the dosage of salmeterol xinafoate is any one of 0.010, 0.020, 0.030, 0.040, 0.050, 0.060, 0.070, 0.080, 0.090, 0.100, 0.110, 0.120, 0.130, 0.140, 0.150, 0.160, 0.170, 0.180, 0.190, 0.200, 0.210, 0.220, 0.230, 0.240, 0.250, 0.260, 0.270, 0.280, 0.290, and 0.300 mg.

In certain embodiments, the medicament is formoterol fumarate. In certain embodiments, the dosage of formoterol fumarate is delivered by any one of the following methods: aerosol, nebulized, powder, metered, and inhalation. In certain embodiments, the dosage of formoterol fumarate is delivered via inhalation. In certain embodiments, the dosage of formoterol fumarate is any one of 0.200, 0.250, 0.300, 0.350, 0.400, 0.450, 0.500, 0.550, 0.600, 0.650, 0.700, 0.750 mg. In certain embodiments, the dosage of formoterol fumarate is 0.450 mg.

In certain embodiments, the medicament is arformoterol. In certain embodiments, the dosage of arformoterol is delivered by any one of the following methods: aerosol, nebulized, powder, metered, and inhalation. In certain embodiments, the dosage of arformoterol is delivered via inhalation. In certain embodiments, the dosage of arformoterol is any one of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, and 75 mg. In certain embodiments, the dosage of arformoterol is 15 mg.

In certain embodiments, the medicament is bambuterol. In certain embodiments, the dosage of bambuterol is delivered by any one of the following methods: aerosol, nebulized, powder, metered, and inhalation. In certain embodiments, the dosage of bambuterol is delivered via inhalation. In certain embodiments, the dosage of arformoterol is any one of 5, 10, 15, 20, 25, 30, 35, 40, 45, 50, 55, 60, 65, 70, 75, 80, 85, 90, 95, and 100 mg.

In certain embodiments, the medicament is clenbuterol. In certain embodiments, the dosage of clenbuterol is delivered by any one of the following methods: aerosol, nebulized, powder, metered, and inhalation.

In certain embodiments, the medicament is formoterol. In certain embodiments, the dosage of formoterol is delivered by any one of the following methods: aerosol, nebulized, powder, metered, and inhalation.

In certain embodiments, the medicament is salmeterol. In certain embodiments, the dosage of salmeterol is delivered by any one of the following methods: aerosol, nebulized, powder, metered, and inhalation.

In certain embodiments, the medicament is protokylol. In certain embodiments, the dosage of protokylol is delivered by any one of the following methods: aerosol, nebulized, powder, metered, and inhalation.

In certain embodiments, the medicament is albuterol. In certain embodiments, the dosage of albuterol is delivered by any one of the following methods: aerosol, nebulized, powder, metered, and inhalation.

In certain embodiments, the medicament is an ultra-long-acting (3 adrenoreceptor agonist (ultra-LABA). In certain embodiments, the ultra-LABA is chosen from indacaterol, olodaterol, and vilanterol.

In certain embodiments, the medicament is indacaterol. In certain embodiments, the dosage of indacaterol is delivered by any one of the following methods: aerosol, nebulized, powder, metered, and inhalation. In certain embodiments, the dosage of indacaterol is delivered via inhalation. In certain embodiments, the dosage of indacaterol is any one of 10, 15, 20, 25, 30, 35, 40, 45, 50, 55, 60, 65, 70, 75, 80, 85, 90, 95, 100, 105, 110, 115, 120, 125, 130, 135, 140, 145, and 150 mg. In certain embodiments, the dosage of indacaterol is 75 mg.

In certain embodiments, the medicament comprises a combined dosage of indacaterol and glycopyrrolate.

In certain embodiments, the medicament is vilanterol. In certain embodiments, the medicament comprises a combined dosage of umeclidinium bromide and vilanterol trifenatate. In certain embodiments, the combined dosage of umeclidinium bromide and vilanterol trifenatate is delivered by any one of the following methods: aerosol, nebulized, powder, metered, and inhalation. In certain embodiments, the combined dosage of umeclidinium bromide and vilanterol trifenatate is delivered via inhalation. In certain embodiments, the medicament comprises a combined dosage of fluticasone furoate and vilanterol trifenatate. In certain embodiments, the combined dosage of fluticasone furoate and vilanterol trifenatate is delivered by any one of the following methods: aerosol, nebulized, powder, metered, and inhalation. In certain embodiments, the combined dosage of fluticasone furoate and vilanterol trifenatate is delivered via inhalation. In certain embodiments, the combined dosage of fluticasone furoate and vilanterol trifenatate contains any one of 0.030, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, and 0.3 mg of fluticasone furoate. In certain embodiments, the combined dosage of fluticasone furoate and vilanterol trifenatate contains any one of 0.010, 0.011, 0.012, 0.013, 0.014, 0.015, 0.016, 0.017, 0.018, 0.019, 0.020, 0.021, 0.022, 0.023, 0.024, 0.025, 0.026, 0.027, 0.028, 0.029, 0.030, 0.031, 0.032, 0.033, 0.034, 0.035, 0.036, 0.037, 0.038, 0.039, 0.040, 0.041, 0.042, 0.043, 0.044, 0.045, 0.046, 0.047, 0.048, 0.049, and 0.050 mg vilanterol trifenatate.

In one aspect, the subject has been exposed to a virus that increases the likelihood or severity of respiratory damage. In one aspect, the subject has been exposed to a virus that increases the likelihood or severity of lung damage. In one aspect, the subject has a genetic predisposition to develop lung fibrosis.

In certain embodiments, the subject has been exposed to a coronavirus (“CoV”). In certain embodiments, the subject has been exposed to a severe acute respiratory syndrome coronavirus (“SARS-CoV”). In certain embodiments, the subject has been exposed to the severe acute respiratory syndrome coronavirus 2 (“SARS-CoV-2”).

In one aspect, the exposure of the subject to the virus is established by the proximity of the subject with a known carrier of the virus. In one aspect, the exposure of the subject to the virus is established by the proximity of the subject with a known carrier of the virus for a period of time.

In one aspect, the exposure of the subject to the virus is confirmed by a positive titer for the virus. In another aspect, the exposure of the subject to the virus is confirmed by a positive titer for antibodies to the virus. In yet another aspect, the exposure of the subject to the virus is confirmed by recognized clinical symptoms associated with the virus.

In certain embodiments, the subject has been exposed to SARS-CoV-2, and the exposure of the subject to the SARS-CoV-2 virus is confirmed by one or more recognized clinical symptoms associated with the SARS-CoV-2 virus. In certain further embodiments, the one or more recognized clinical symptoms associated with the SARS-CoV-2 virus include one or more of the following: fever, chills, shortness of breath, dyspnea, tremors, fatigue, myalgia, headache, taste dysfunction, and smell dysfunction. In a further aspect, the subject has experienced depressed pulmonary activity. The depressed pulmonary activity may be manifested by a decreased blood oxygenation level, the need for administering supplemental oxygen, or the need for a respirator.

In some aspects of the present disclosure, after treatment, the subject experiences a substantial reduction in the rate of pulmonary fibrosis progression compared to pre-treatment. This reduction can be quantified by objective measures (chest X-ray, pulmonary function tests, etc.).

In some aspects of the present disclosure, after treatment, the subject experiences a substantial reduction in the hospitalization rate based on improved breathlessness, dyspnea, or cough status. In some embodiments, after the treatment, the patient experiences a substantial reduction of cough characterized by at least a one-point reduction in the cough severity Numerical Rating Scale (NRS) value compared to before treatment. In some embodiments, the reduction of cough is characterized by a decline in NRS cough value ranging from about 1.0 to about 9.0 points, for example, about 1.0 point, about 2.0 point, about 3.0 points, about 4.0 points, about 5.0 points, about 6.0 points, about 7.0 points, about 8.0 points, about 9.0 points and about 10.0 points compared to before treatment.

In some aspects of the present disclosure, after treatment, the subject experiences a substantial improvement in health status related to a reduction in cough frequency characterized by at least about a 1.0-point improvement in the total score on the patient's Leicester Cough Questionnaire (LCQ) score compared to before treatment. In some embodiments, the improvement in health status related to a reduction of cough frequency is characterized by an improvement in Leicester Cough Questionnaire score ranging from about 0.5 to about 2.0 points, for example, about 0.5 points, about 1.0 point, about 1.5 points, and about 2.0 points compared to before treatment. In some embodiments, the improvement in health status related to the reduction of cough frequency is characterized by an improvement in any of the three Leicester Cough Questionnaire domains (physical, psychological or social) score ranging from about 0.5 to about 2.0 points, for example, about 0.5 points, about 1.0 point, about 1.5 points, and about 2.0 points compared to before treatment.

In some aspects of the present disclosure, after the treatment, the subject experiences a substantial reduction of fatigue compared to before treatment. In some embodiments, after said treatment, the patient experiences a reduction of fatigue characterized by at least one category change in at least one of the seven questions of the PROMIS Item Bank v1.0 Fatigue Short Form 7a Scale. In some embodiments, the reduction of fatigue is characterized by an improvement in at least one of the seven questions of the PROMIS Item Bank v1.0 Fatigue Short Form 7a Scale ranging from at least one category to about three categories, for example, about one category, about two categories, about three categories, about four categories, about five categories, about six categories, and about seven categories compared to before treatment.

In some aspects of the present disclosure, the subject experiences a substantial reduction in the frequency and/or severity of breathlessness episodes after the treatment. In some embodiments, after treatment, the patient experiences a reduction of breathlessness characterized by at least a 1.0-point reduction in the Evaluating Respiratory Symptoms (E-RS™) breathlessness subscale score compared to before treatment. In some embodiments, the reduction of breathlessness is characterized by a decline in Evaluating Respiratory Symptoms (E-RS™) breathlessness subscale score ranging from about 1.0 to about 23.0 points (Bacci E D, O'Quinn S, Leidy N K, Murray L, Vernon M. Evaluation of a respiratory symptom diary for clinical studies of idiopathic pulmonary fibrosis. Respir Med. 2018 January; 134:130-138), for example, about 1.0 point, about 3.0 points, about 5.0 points, about 7.0 points, about 9.0 points, about 11.0 points, about 13.0 points, about 15.0 points, about 17.0 points, about 19 points, about 21 points and about 23.0 points compared to before treatment.

In some aspects of the present disclosure, after the treatment, the subject experiences a substantial reduction in the frequency and/or severity of dyspnea episodes. In some embodiments, after said treatment, the patient experiences a reduction of dyspnea characterized by at least a one-point change in the Borg dyspnea scale value total score compared to before treatment. In some embodiments, the reduction of dyspnea is characterized by a decline in Borg dyspnea scale value total score ranging from about 0.5 to about 2.0 points, for example, about 0.5 points, about 1.0 point, about 1.5 points, and about 2.0 points compared to before treatment.

In some aspects of the present disclosure, after the treatment, the patient experiences a substantial reduction in the hospitalization rate based on improvement in breathlessness, dyspnea, or cough status.

### Abbreviations and Definitions

To aid understanding of the disclosure, many terms and abbreviations as used herein are defined below as follows:

When introducing elements of the present disclosure or the preferred embodiment(s) thereof, the articles “a,” “an,” “the,” and “said” are intended to mean that there are one or more of the elements. The terms “comprising,” “including,” and “having” are intended to be inclusive and mean that there may be additional elements other than the listed elements.

The term “and/or” when used in a list of two or more items means that any one of the listed items can be employed by itself or in combination with any one or more of the listed items.

For example, the expression “A and/or B” is intended to mean either or both of A and B, i.e., A alone, B alone, or A and B in combination. The expression “A, B, and/or C” means A alone, B alone, C alone, A and B in combination, A and C in combination, B and C in combination, or A, B, and C in combination.

When ranges of values are disclosed, and the notation “from n1 . . . to n2” or “between n1 . . . and n2” is used, where n1 and n2 are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein in relation to a numerical value x means x±10%.

The term “disease” as used herein is intended to be generally synonymous. It is used interchangeably with the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means administering two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. Also, such administration encompasses the use of each type of therapeutic agent in a sequential manner. In either case, the treatment regimen will benefit the drug combination in treating the conditions or disorders described herein.

The phrase “therapeutically effective” or “effective amount” is intended to qualify the amount of active ingredients used in treating a disease or disorder or on the effecting of a clinical endpoint.

The term “therapeutically acceptable” refers to those compounds (or salts, prodrugs, tautomers, zwitterionic forms, etc.) which are suitable for use in contact with the tissues of subjects without undue toxicity, irritation, and allergic response, are commensurate with a reasonable benefit/risk ratio and are effective for their intended use.

Reference to “treatment” of a subject is intended to include prevention, prophylaxis, attenuation, amelioration, and therapy. Treatment may also include the prevention of disease. Prevention of a disease may involve complete protection from disease, such as preventing infection with a pathogen, or may involve prevention of disease progression. For example, prevention of a disease may not mean complete foreclosure of any effect related to the disease at any level. Instead, it may mean preventing the symptoms of a disease to a clinically significant or detectable level. The prevention of diseases may also mean preventing disease progression to a later stage of the disease.

The terms “subject” or “patient” are used interchangeably herein to mean all mammals, including humans. In certain embodiments, the subject is a human. In certain embodiments, the subject is a human or mammal. In certain embodiments, the subject is chosen from a human and mammal. Examples of subjects include, but are not limited to, humans, monkeys, dogs, cats, horses, cows, goats, sheep, pigs, and rabbits.

The terms “affected with a disease or disorder,” “afflicted with a disease or disorder,” or “having a disease or disorder” are used interchangeably herein and refer to a subject with any disease, disorder, syndrome, or condition. One of the terms implies no increased or decreased level of severity of the disorder compared to the other.

The compounds disclosed herein can exist as therapeutically acceptable salts. The present disclosure includes compounds listed above in the form of salts, including acid addition salts. Suitable salts include those formed with both organic and inorganic acids. Such acid addition salts will normally be pharmaceutically acceptable. However, salts of non-pharmaceutically acceptable salts may be of utility in preparing and purifying the compound in question. Basic addition salts may also be formed and be pharmaceutically acceptable. For a complete discussion of the preparation and selection of salts, refer to Pharmaceutical Salts: Properties, Selection, and Use (Stahl, P. Heinrich. Wiley-VCHA, Zurich, Switzerland, 2002).

The term “pulmonary disease” refers to diseases affecting the organs involved in breathing, including but not limited to the nose, throat, larynx, Eustachian tubes, trachea, bronchi, lungs, related muscles, and nerves. Pulmonary diseases include, but are not limited to, asthma, adult respiratory distress syndrome, and allergic (extrinsic) asthma, non-allergic (intrinsic) asthma, acute severe asthma, chronic asthma, clinical asthma, nocturnal asthma, allergen-induced asthma, aspirin-sensitive asthma, exercise-induced asthma, isocapnic hyperventilation, child-onset asthma, adult-onset asthma, cough-variant asthma, occupational asthma, steroid-resistant asthma, seasonal asthma, seasonal allergic rhinitis, perennial allergic rhinitis, chronic obstructive pulmonary disease, including chronic bronchitis or emphysema, pulmonary hypertension, interstitial lung fibrosis and/or airway inflammation and cystic fibrosis, and hypoxia.

The term “sequela” (plural “sequelae”) refers to a disease or symptoms that affect a subject as a consequence of the subject having experienced a disease, infection, illness, or injury. In some aspects, a sequela is diagnosed no earlier than 1, 2, 3, 6, 12, 18, or 24 months after the disease, infection, illness, or injury. In some aspects, a sequela is diagnosed no later than 6, 12, 18, 24, or 30 months after the disease, infection, illness, or injury.

The terms “fibrosis” and “fibrosing disorder” refer to conditions that are associated with the abnormal accumulation of cells and/or fibronectin and/or collagen and/or increased fibroblast recruitment and include but are not limited to fibrosis of individual organs or tissues such as the heart, kidney, liver, joints, lung, pleural tissue, peritoneal tissue, skin, cornea, retina, musculoskeletal and digestive tract.

Exemplary diseases, disorders, or conditions that involve fibrosis include, but are not limited to lung diseases associated with fibrosis, including but not limited to idiopathic pulmonary fibrosis, pulmonary fibrosis secondary to systemic inflammatory disease such as rheumatoid arthritis, scleroderma, lupus, cryptogenic fibrosing alveolitis, radiation induced fibrosis, chronic obstructive pulmonary disease (COPD), scleroderma, chronic asthma, silicosis, asbestos induced pulmonary or pleural fibrosis, acute lung injury and acute respiratory distress (including bacterial pneumonia induced, trauma induced, viral pneumonia induced, ventilator induced, non-pulmonary sepsis induced, and aspiration induced); chronic nephropathies associated with injury/fibrosis (kidney fibrosis), including but not limited to glomerulonephritis secondary to systemic inflammatory diseases such as lupus and scleroderma, diabetes, glomerular nephritis, focal segmental glomerular sclerosis, IgA nephropathy, hypertension, allograft and Alport syndrome; gut fibrosis, including but not limited to scleroderma, and radiation induced gut fibrosis; liver fibrosis, including but not limited to cirrhosis, alcohol induced liver fibrosis, nonalcoholic steatohepatitis (NASH), biliary duct injury, primary biliary cirrhosis, infection or viral induced liver fibrosis (e.g., chronic HCV infection), and autoimmune hepatitis; head and neck fibrosis, e.g., radiation induced; corneal scarring, e.g., LASIK (laser-assisted in situ keratomileusis), corneal transplant, and trabeculectomy; hypertrophic scarring and keloids, e.g., burn induced or surgical; and other fibrotic diseases, e.g., sarcoidosis, scleroderma, spinal cord injury/fibrosis, myelofibrosis, vascular restenosis, atherosclerosis, arteriosclerosis, Wegener's granulomatosis, mixed connective tissue disease, and Peyronie's disease.

The term “therapeutically acceptable salt” represents salts or zwitterionic forms of the compounds disclosed herein, which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids that can be employed to form therapeutically acceptable addition salts include inorganic acids such as hydrochloric, hydrobromic, sulfuric, phosphoric, and organic acids such as oxalic, maleic, succinic, and citric. Salts can also be formed by coordinating the compounds with an alkali metal or alkaline earth ion. Hence, the present disclosure contemplates sodium, potassium, magnesium, and calcium salts of the compounds disclosed herein and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for forming base addition salts include ethylenediamine, ethanolamine, diethanolamine, piperidine, and piperazine.

A salt of a compound can be made by reacting the appropriate compound as the free base with the appropriate acid.

### Pharmaceutical Formulations

While it may be possible for the compounds disclosed herein to be administered as the raw chemical, it is also possible to present them as a pharmaceutical formulation. Accordingly, provided herein are pharmaceutical formulations which comprise one or more of certain compounds disclosed herein, or one or more pharmaceutically acceptable salts, esters, prodrugs, amides, or solvates thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and understood in the art, e.g., in Remington's Pharmaceutical Sciences. The pharmaceutical compositions disclosed herein may be manufactured in any manner known in the art, e.g., through conventional mixing, dissolving, granulating, dragee-making, levigating, emulsifying, encapsulating, entrapping, or compression processes.

The formulations include those suitable for oral, parenteral (including subcutaneous, intradermal, intramuscular, intravenous, intraarticular, and intramedullary), intraperitoneal, transmucosal, transdermal, rectal, and topical (including dermal, buccal, sublingual, and intraocular) administration. However, the most suitable route may depend upon, for example, the condition and disorder of the recipient. The formulations may conveniently be presented in unit dosage form and may be prepared by any of the methods well known in the art of pharmacy. Typically, these methods include the step of bringing into association a compound of the subject disclosure or a pharmaceutically acceptable salt, ester, amide, prodrug, or solvate thereof (“active ingredient”) with the carrier, which constitutes one or more accessory ingredients. In general, the formulations are prepared by uniformly and intimately bringing into association the active ingredient with liquid carriers or finely divided solid carriers or both and then, if necessary, shaping the product into the desired formulation.

Fillers include, but are not limited to, lactose, saccharose, glucose, starch, microcrystalline cellulose, microfine cellulose, mannitol, sorbitol, calcium hydrogen phosphate, aluminum silicate, amorphous silica, and sodium chloride, starch, and dibasic calcium phosphate dihydrate. In one embodiment, the filler is not water-soluble, although it may absorb water. In one embodiment, the filler is a spheronization aid. Spheronization aids can include one or more crospovidone, carrageenan, chitosan, pectinic acid, glycerides, β-cyclodextrin (β-CD), cellulose derivatives, microcrystalline cellulose, powdered cellulose, polyplasdone crospovidone, and polyethylene oxide.

Binders include, but are not limited to, cellulose ethers, methylcellulose, ethylcellulose, hydroxyethylcellulose, propyl cellulose, hydroxypropyl cellulose, lower-substituted hydroxypropyl cellulose, hydroxypropylmethylcellulose (hypromellose, e.g., hypromellose 2910, Methocel™ E), carboxymethyl cellulose, starch, pregelatinized starch, acacia, tragacanth, gelatin, polyvinyl pyrrolidone (povidone), cross-linked polyvinyl pyrrolidone, sodium alginate, microcrystalline cellulose, and lower-alkyl-substituted hydroxypropyl cellulose. In one embodiment, the binders are selected from wet binders.

Surfactants include, but are not limited to, anionic surfactants, including sodium lauryl sulfate, sodium deoxycholate, dioctyl sodium sulfosuccinate, and sodium stearyl fumarate, nonionic surfactants, including polyoxyethylene ethers and polysorbate 80, and cationic surfactants, including quaternary ammonium compounds. In one embodiment, the surfactant is selected from anionic surfactants, e.g., sodium lauryl sulfate.

Disintegrants include, but are not limited to, starch, sodium cross-linked carboxymethyl cellulose, carmellose sodium, carmellose calcium, cross-linked polyvinyl pyrrolidone, and sodium starch glycolate, low-substituted hydroxypropyl cellulose, and hydroxypropyl starch.

Glidants include, but are not limited to, polyethylene glycols of various molecular weights, magnesium stearate, calcium stearate, calcium silicate, fumed silicon dioxide, magnesium carbonate, magnesium lauryl sulfate, aluminum stearate, stearic acid, palmitic acid, cetanol, stearol, and talc.

Lubricants include, but are not limited to, stearic acid, magnesium stearate, calcium stearate, aluminum stearate, and siliconized talc

In certain embodiments, the formulation further comprises one or more antioxidants. Examples of pharmaceutically-acceptable antioxidants include (1) water-soluble antioxidants, such as ascorbic acid, cysteine hydrochloride, sodium bisulfate, sodium metabisulfite, sodium sulfite, and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediaminetetraacetic acid (EDTA), sorbitol, tartaric acid, phosphoric acid, and the like.

### Routes of Administration

Oral Administration

The compounds of the present disclosure may be administered orally, including swallowing, so the compound enters the gastrointestinal tract or is absorbed into the bloodstream directly from the mouth, including sublingual or buccal administration.

Suitable compositions for oral administration include solid formulations such as tablets, pills, cachets, lozenges, and hard or soft capsules, containing liquids, gels, powders, or granules.

In a tablet or capsule dosage form, the amount of drug present may be from about 0.05% to about 95% by weight, more typically from about 2% to about 50% by weight of the dosage form.

Also, tablets or capsules may contain a disintegrant, comprising from about 0.5% to about 35% by weight, more typically from about 2% to about 25% of the dosage form. Examples of disintegrants include methylcellulose, sodium or calcium carboxymethyl cellulose, croscarmellose sodium, polyvinylpyrrolidone, hydroxypropyl cellulose, starch, and the like.

Suitable binders for use in a tablet include gelatin, polyethylene glycol, sugars, gums, starch, hydroxypropyl cellulose, and the like. Suitable diluents for use in a tablet include mannitol, xylitol, lactose, dextrose, sucrose, sorbitol, and starch.

Suitable surface-active agents and glidants for use in a tablet or capsule may be present in amounts from about 0.1% to about 3% by weight and include polysorbate 80, sodium dodecyl sulfate, talc, and silicon dioxide.

Suitable lubricants for use in a tablet or capsule may be present in amounts from about 0.1% to about 5% by weight and include calcium, zinc or magnesium stearate, sodium stearyl fumarate, and the like.

Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, lubricating, surface active, or dispersing agents. Molded tablets may be made by molding a mixture of the powdered compound moistened with a liquid diluent in a suitable machine. Dyes or pigments may be added to tablets to identify or characterize different combinations of active compound doses.

Liquid formulations can include emulsions, solutions, syrups, elixirs, and suspensions, which can be used in soft or hard capsules. Such formulations may include a pharmaceutically acceptable carrier, for example, water, ethanol, polyethylene glycol, cellulose, or an oil. The formulation may also include one or more emulsifying agents and/or suspending agents.

Compositions for oral administration may be formulated as an immediate or modified release, including delayed or sustained release, optionally with enteric coating.

In another embodiment, a pharmaceutical composition comprises a therapeutically effective amount of a compound of Formula (I) or a pharmaceutically acceptable salt thereof and a pharmaceutically acceptable carrier.

Parenteral Administration

Compounds of the present disclosure may be administered directly into the bloodstream, muscle, or internal organs by injection, e.g., by bolus injection or continuous infusion. Suitable means for parenteral administration include intravenous, intramuscular, subcutaneous intraarterial, intraperitoneal, intrathecal, intracranial, and the like. Suitable devices for parenteral administration include injectors (including needle and needle-free injectors) and infusion methods. The formulations may be presented in unit-dose or multi-dose containers, for example, sealed ampoules and vials.

Most parenteral formulations are aqueous solutions containing excipients, including salts, buffering, suspending, stabilizing, and/or dispersing agents, antioxidants, bacteriostats, preservatives, and solutes which render the formulation isotonic with the blood of the intended recipient and carbohydrates.

Parenteral formulations may also be prepared in a dehydrated form (e.g., by lyophilization) or sterile non-aqueous solutions. These formulations can be used with a suitable vehicle, such as sterile water. Solubility-enhancing agents may also be used for preparing parenteral solutions.

Compositions for parenteral administration may be formulated as an immediate or modified release, including delayed or sustained release. Compounds may also be formulated as depot preparations. Such long-acting formulations may be administered by implantation (for example, subcutaneously or intramuscularly) or intramuscular injection. Thus, for example, the compounds may be formulated with suitable polymeric or hydrophobic materials (for example, as an emulsion in an acceptable oil) or ion exchange resins, or as sparingly soluble derivatives, for example, as a sparingly soluble salt.

Methods of Delivery

This disclosure contemplates the following modes of delivery:

PEG (polyethylene glycol (PEG) or PEGylated, or particulate form as used and defined nicotine or cannabis electronic cigarettes (e-cig or vaping), including but not limited to salts of the therapeutic components such as protonated forms of the therapeutics disclosed containing the pharmacologic agent of interested taken from any dose or combination of corticosteroid, beta2 adrenoreceptor agonist and tyrosine kinase antagonist listed above, in this disclosure.

Soft Mist inhaler, nebulizer, HFA (hydrofluoroalkane metered-dose inhaler with or without a spacer, Vaping device consisting of an atomizer, a power source such as a self-contained battery, and a container such as a cartridge or a tank, whereby instead of the user inhaling smoke, the user inhales a vapor form of the therapeutic and delivery vehicle contained or administered within said container.

A salt liquid formulation containing the pharmacologic agent of interest taken from any dose or combination of corticosteroid, beta2 adrenoreceptor agonist, and tyrosine kinase antagonist listed above, in this disclosure, for generating an inhalable aerosol in an electronic cigarette comprising a salt that forms about 0.5% to about 50% pharmacologic agent is provided. Provided herein is a method of delivering the pharmacologic agent to a user comprising operating an electronic cigarette to a user wherein the electronic cigarette comprises a salt of the pharmacologic agent formulation comprising a pharmacologic agent salt in a biologically acceptable liquid carrier wherein an acid used to form said pharmacologic agent salt is characterized by vapor pressure >20 mmHg at 200° C., and inhaling an aerosol generated from the pharmacologic agent salt formulation heated by the electronic cigarette.

A method of delivering a pharmacologic “agent” consisting of any dose of any combination of corticosteroid, beta2 adrenoreceptor agonist and tyrosine kinase antagonist listed above, in this disclosure, to a user comprising deploying an electronic cigarette comprising a formulation comprising said “agent” in a biologically acceptable liquid carrier, wherein the operation of the electronic cigarette generates an inhalable aerosol.

An aspect of the present invention relates to a substantially pure conjugate containing one or more polymer moieties, a protein moiety, and a linker to the pharmacologic agent of interest taken from any dose or combination of corticosteroid beta2 adrenoreceptor agonist and tyrosine kinase antagonist listed above, in this disclosure. In the conjugate, the polymer moiety or moieties are attached to the linker; the nitrogen atom of the N-terminus of the pharmacologic moiety is bonded to the linker; the linker is a covalent bond, C1-10 alkylene, C2-10 alkenylene, or C2-10 alkynylene; and the pharmacologic moiety consists of any combination of corticosteroid, beta2 adrenoreceptor agonist and tyrosine kinase antagonist listed above, in this disclosure. This conjugate has an unexpected long in vivo half-life.

Topical Administration

Compounds of the present disclosure may be administered topically (for example, to the skin, mucous membranes, ear, nose, or eye) or transdermally. Formulations for topical administration can include, but are not limited to, lotions, solutions, creams, gels, hydrogels, ointments, foams, implants, patches, and the like. Carriers that are pharmaceutically acceptable for topical administration formulations can include water, alcohol, mineral oil, glycerin, polyethylene glycol, and the like. Topical administration can also be performed by, for example, electroporation, iontophoresis, phonophoresis, and the like.

Typically, the active ingredient for topical administration may comprise from 0.001% to 10% w/w (by weight) of the formulation. In certain embodiments, the active ingredient may comprise as much as 10% w/w; less than 5% w/w; from 2% w/w to 5% w/w; or from 0.1% to 1% w/w of the formulation.

Compositions for topical administration may be formulated as an immediate or modified release, including delayed or sustained release.

Rectal, Buccal, and Sublingual Administration

Suppositories for rectal administration of the compounds of the present disclosure can be prepared by mixing the active agent with a suitable non-irritating excipient such as cocoa butter, synthetic mono-, di-, or triglycerides, fatty acids, or polyethylene glycols which are solid at ordinary temperatures but liquid at the rectal temperature, and which will therefore melt in the rectum and release the drug.

For buccal or sublingual administration, the compositions may take the form of tablets, lozenges, pastilles, or gels formulated conventionally. Such compositions may comprise the active ingredient in a flavored basis, such as sucrose and acacia or tragacanth.

Administration by Inhalation

For administration by inhalation, compounds may be conveniently delivered from an insufflator, nebulizer pressurized packs, or other convenient means of delivering an aerosol spray or powder. Pressurized packs may comprise a suitable propellant such as dichlorodifluoromethane, trichlorofluoromethane, dichlorotetrafluoroethane, carbon dioxide, or other suitable gas. In a pressurized aerosol, the dosage unit may be determined by providing a valve to deliver a metered amount. Alternatively, for administration by inhalation or insufflation, the compounds according to the disclosure may take the form of dry powder composition, for example, a powder mix of the compound and a suitable powder base such as lactose or starch. The powder composition may be presented in a unit dosage form, such as capsules, cartridges, gelatin, or blister packs from which the powder may be administered with the aid of an inhalator or insufflator.

Other carrier materials and modes of administration known in the pharmaceutical art may also be used. Pharmaceutical compositions of the disclosure may be prepared by any of the well-known techniques of pharmacy, such as effective formulation and administration procedures. Unit dosage formulations contain an effective dose, as herein recited, or an appropriate fraction thereof, of the active ingredient. The precise amount of compound administered to a subject will be the responsibility of the attendant physician. The specific dose level for any particular subject will depend upon a variety of factors, including the activity of the specific compound employed, the age, body weight, general health, sex, diets, time of administration, route of administration, rate of excretion, drug combination, the precise disorder being treated, and the severity of the indication or condition being treated. Also, the route of administration can and will vary depending on the condition and its severity. The above considerations concerning effective formulations and administration procedures are well known in the art and are described in standard textbooks. Formulation of drugs is discussed in, for example, Hoover, John E., Remington's Pharmaceutical Sciences, Mack Publishing Co., Easton, Pa., 1975; Liberman, et al., Eds., Pharmaceutical Dosage Forms, Marcel Decker, New York, N.Y., 1980; and Kibbe, et al., Eds., Handbook of Pharmaceutical Excipients (3^{rd} Ed.), American Pharmaceutical Association, Washington, 1999.

### Methods of Treatment

The methods of the disclosure can be used to treat any subject in need of treatment.

In various embodiments, the disclosed methods are used (1) to treat immediately (acutely) so that long term sequelae are reduced, (2) to treat continuously (maintenance therapy) for a fixed or defined time or for an extended and undefined time period, or (3) to treat symptomatically (as needed) after viral recovery. The severity of the disease has been correlated with the long-term outcomes, regardless of the SARS2 variant.

Examples of subjects include, but are not limited to, humans, monkeys, deer, camel, pets, and companion animals, including, but not limited to, dogs, cats, horses, rabbits, and guinea pigs; livestock, including, but not limited to, cows, buffalo, bison, mules, goats, sheep, and pigs. In one embodiment, the subject is a human.

Asthma

In certain embodiments, the methods disclosed herein are used to treat asthma in a subject which is a consequence of previous exposure of the subject to a virus, particularly SARS-CoV-2. The inflammation associated with asthma represents exaggerated repair processes which can, themselves, lead to airway damage.

In one aspect, in the asthmatic individual, the release of normal repair mediators, including lysophosphatidic acid (LPA), is exaggerated. The actions of the repair mediators are inappropriately prolonged, leading to airway damage. In some aspects, the airway damage associated with asthma may include a thickened lamina reticularis, increased count and activity of myofibroblasts, increased numbers and secretory activity of mucus glands, thickening of the smooth muscle layer, and altered morphology of both the connective tissue and capillary bed throughout the airway wall. Thickening of the airway wall can, in turn, constrict the lumen of the asthmatic airway, thus decreasing airflow.

Fibrosis

In certain embodiments, the methods disclosed herein are used to treat fibrosis in a subject, resulting from previous exposure of the subject to a virus, particularly SARS-CoV-2. Tissue injury initiates a complex series of host wound-healing responses. Failure of these responses, which otherwise would restore normal tissue structure and function, can lead to tissue fibrosis and loss of function. Several events and factors play roles in developing fibrosis in organs, including without limitation the lungs. Molecules involved in fibrosis development include proteins or peptides, such as cytokines, chemokines, metalloproteinases, and phospholipids.

In the lung, failure of the wound healing responses can contribute to the pathogenesis of fibrotic lung diseases. Fibrotic lung diseases, such as idiopathic pulmonary fibrosis (IPF), are associated with high morbidity and mortality.

Fibrosis can also manifest in related respiratory ailments. Peribronchiolar fibrosis can lead to chronic bronchitis, emphysema, and interstitial lung disease. Fibrosis of the alveolar wall is associated with emphysema. Other manifestations of fibrosis include fibrotic interstitial lung diseases and obliterative bronchiolitis, and chronic obstructive pulmonary disease.

Pulmonary fibrosis involves the hardening and scarring of lung tissue due to excess deposition of extracellular matrix components, including collagen, by fibroblasts. Fibroblasts participate in inflammation and immune cell recruitment to sites of tissue injury, and both produce and can respond to inflammatory cytokines.

The diagnosis of pulmonary fibrosis can usually be made by a careful history, including physical examination, chest radiography, including a high-resolution computer tomographic scan (HRCT), and open lung or transbronchial biopsies. Histologic examination of tissue obtained at open lung biopsy allows classification of these patients into several categories, including Usual Interstitial Pneumonia (UIP), Desquamative Interstitial Pneumonia (DIP), and Non-Specific Interstitial Pneumonia (NSIP).

In one aspect, a method of the present disclosure comprises the administration of a compound as disclosed herein or a pharmaceutically acceptable salt to a subject. In one aspect, the aforementioned method is directed at treating fibrosis of an organ or tissue in a subject, preferentially the lungs or pulmonary tissue. One aspect is a method for preventing a fibrosis condition in a subject. The method comprises administering to the subject at risk of developing one or more fibrosis conditions a therapeutically effective amount of a compound as disclosed herein or a pharmaceutically acceptable salt thereof.

### Combinations and Combination Therapy

The compounds of the present disclosure can be used, alone or in combination with other pharmaceutically active compounds, to treat conditions such as those disclosed hereinabove. The compound(s) of the present disclosure and other pharmaceutically active compound(s) can be administered simultaneously (either in the same dosage form or in separate dosage forms) or sequentially. Accordingly, in one embodiment, the present disclosure comprises methods for treating a condition by administering to the subject a therapeutically-effective amount of one or more compounds of the present disclosure and one or more additional pharmaceutically active compounds.

In some embodiments, the method of the current disclosure includes the coadministration of at least one antitussive, anti-breathlessness, and anti-dyspneic drug.

In some embodiments, the method of the current disclosure includes the coadministration to the subject with fibrosis, or with a predisposition of developing fibrosis, with one or more other agents that are used to treat fibrosis. In one aspect, the one or more agents include corticosteroids. In one aspect, the one or more agents include immunosuppressants. In one aspect, the one or more agents include B-cell antagonists. In one aspect, the one or more agents include uteroglobin.

In another embodiment, there is provided a pharmaceutical composition comprising one or more compounds of the present disclosure, one or more additional pharmaceutically active compounds, and a pharmaceutically acceptable carrier.

The methods disclosed herein can also include coadministration with other therapeutic reagents selected for their therapeutic value for the condition to be treated. In general, the compounds described herein and, in embodiments where combination therapy is employed, other agents do not have to be administered in the same pharmaceutical composition. Because of different physical and chemical characteristics, they are optionally administered by different routes. The initial administration is generally made according to established protocols and then, based upon the observed effects, the dosage, modes of administration, and times of administration subsequently modified. The subject's overall benefit is either simply additive of the two therapeutic agents, or the subject experiences an enhanced (i.e., synergistic) benefit. Alternatively, if a compound disclosed herein has a side effect, it may be appropriate to administer an agent to reduce the side effect; or the therapeutic effectiveness of a compound described herein may be enhanced by administration of an adjuvant.

Therapeutically effective dosages vary when the drugs are used in treatment combinations. Methods for experimentally determining therapeutically-effective dosages of drugs and other agents for use in combination treatment regimens are documented methodologies. Combination treatment further includes periodic treatments that start and stop at various times to assist with the clinical management of the subject. In any case, the multiple therapeutic may be administered in any order or simultaneously. If simultaneously, the multiple therapeutic agents are optionally provided in a single, unified form, or multiple forms (by way of example only, either as a single pill or as two separate pills).

In some embodiments, one of the therapeutic agents is given in multiple doses, or both are given multiple doses. If not simultaneous, the timing between the multiple doses optionally varies from more than zero weeks to less than twelve weeks.

Also, the combination methods, compositions, and formulations are not limited to the use of only two agents. The use of multiple therapeutic combinations is also envisioned. It is understood that the dosage regimen to treat, prevent, or ameliorate the condition(s) for which relief is sought is optionally modified per various factors. These factors include the disorder from which the subject suffers and the subject's age, weight, sex, diet, and medical condition. Thus, the dosage regimen actually employed varies widely, in some embodiments, and therefore deviates from the dosage regimens set forth herein.

The pharmaceutical agents which make up the combination therapy disclosed herein are optionally a combined dosage form or in separate dosage forms intended for substantially simultaneous administration. The pharmaceutical agents that make up the combination therapy are optionally also administered sequentially, with either agent being administered by a regimen calling for two-step administration. The two-step administration regimen optionally calls for sequential administration of the active agents or spaced-apart administration of the separate active agents. The time between the multiple administration steps ranges from a few minutes to several hours, depending upon the properties of each pharmaceutical agent, such as potency, solubility, bioavailability, plasma half-life, and kinetic profile of the pharmaceutical agent.

Where a subject is suffering from or at risk of suffering from an inflammatory condition, the methods as disclosed herein may include the coadministration of one or more agents or methods for treating an inflammatory condition in any combination. Therapeutic agents/treatments for treating an autoimmune and/or inflammatory condition include, but are not limited to any of the following examples: (1) corticosteroids, including but not limited to cortisone, dexamethasone, and methylprednisolone; (2) nonsteroidal anti-inflammatory drugs (NSAIDs), including but not limited to ibuprofen, naproxen, acetaminophen, aspirin, fenoprofen (NALFON), flurbiprofen (ANSAID), ketoprofen, oxaprozin (DAYPRO), diclofenac sodium (VOLTAREN), diclofenac potassium (CATAFLAM), etodolac (LODINE), indomethacin (INDOCIN), ketorolac (TORADOL), sulindac (CLINORIL), tolmetin (TOLECTIN), meclofenamate (MECLOMEN), mefenamic acid (PONSTEL), nabumetone (RELAFEN) and piroxicam (FELDENE); (3) immunosuppressants, including but not limited to methotrexate (RHEUMATREX), leflunomide (ARAVA), azathioprine (IMURAN), cyclosporine (NEORAL, SANDIMMUNE), tacrolimus and cyclophosphamide (CYTOXAN); (4) CD20 blockers, including but not limited to rituximab (RITUXAN); (5) Tumor Necrosis Factor (TNF) blockers, including but not limited to etanercept (ENBREL), infliximab (REMICADE) and adalimumab (HUMIRA); (6) interleukin-1 receptor antagonists, including but not limited to anakinra (KINERET); (7) interleukin-6 inhibitors, including but not limited to tocilizumab (ACTEMRA); (8) interleukin-17 inhibitors, including but not limited to AIN457; (9) Janus kinase inhibitors, including but not limited to tasocitinib; and (10) syk inhibitors, including but not limited to fostamatinib.

### Biological and Clinical Assays

The following are examples of biological and clinical assays useful with the methods of the disclosure. The assays provided herein are not limiting, and other assays are now known, or later discovered, by one of skill in the art can be used for the same purpose as the assays provided below.

Intratracheal inoculation of bleomycin Eight to ten-week-old male C57BL/6 mice weighing 24 to 28 g are used for the experiments. After measuring their body weight, the mice are anesthetized with an intraperitoneal injection of avertin. The mice's trachea is exposed by a 1.0 cm longitudinal incision in the neck and injected with 55 μL of a bleomycin hydrochloride solution containing 1.5 mg or 2.0 mg of bleomycin dissolved in a sterile phosphate-buffered saline solution per kilogram of body weight. The test mice are treated with 50 μg alloferon intraperitoneally daily from the day of bleomycin inoculation. All procedures are conducted in a sterile environment and are reviewed and approved by the appropriate Institutional Review Board.

Histopathological scoring Mice are euthanized with CO₂ asphyxiation. After thoracotomy, the lungs are perfused with saline via the right ventricle and inflated with 2 mL of phosphate-buffered 4% paraformaldehyde solution via the trachea and fixed for 24 hours. Routine light microscopic techniques are performed for paraffin embedding, and the sections are stained with H&E and Masson's trichrome.

Bronchoalveolar (BAL) cell counting Mice are sacrificed by asphyxiation in a CO₂ chamber. The mice are dampened with 70% ethanol in a biosafety cabinet. The mice are then placed front side up on a Styrofoam panel, and the arms and legs of the mice are fixed with needles or tape. Scissors are used to make an incision in the skin from the abdomen to the neck, and the skin retracted with forceps to expose the thoracic cage and neck. The muscle around the neck is gently removed to expose the trachea. Forceps are used to put an approximately 10 cm-long nylon string under the trachea. The ribs are then cut to expose the heart and the lungs without cutting the trachea and lungs. A 22G×1 in. Exel Safelet Catheter is inserted into the trachea, the stylet hub removed, and the catheter and the trachea are tied together firmly with the nylon string.

A 1-mL syringe is loaded with 0.8 ml of phosphate-buffered saline (PBS) and placed at the end of the catheter. The PBS is injected and aspirated four times. The syringe is then removed from the catheter, and the recovered lavage fluid is saved in 1.5 mL Eppendorf tubes on ice. The BAL volume is recorded according to the scales on the 1.5 mL Eppendorf tubes. The BAL fluid is centrifuged at 800 g for 10 min at 4° C. After centrifugation, the supernatant is transferred to a new 5 ml tube, with a protease inhibitor cocktail added to a final concentration of 1× and PMSF to a final concentration of 1 mM and mixed well. The BAL cell pellets are resuspended in 400 μL of PBS, and the cells are counted by taking about 20 μL of the cell sample to a hemocytometer and counting the cells under a microscope.

Collagen content measurement Lung tissue is homogenized in 100 μL ddH2O. To a 100 μL of sample homogenate, 100 μL concentrated HCl C12 M) is added in a pressure-tight Teflon capped vial. The samples are hydrolyzed at 120° C. for 3 hrs. After homogenization, the samples are clarified by adding 4 mg of activated charcoal. The samples are then vortexed and centrifuged at 10,000 g for 3 min to remove the precipitate and activated charcoal. 10-30 μL of each hydrolyzed sample is transferred to a 96-well plate and evaporated to dryness under vacuum/on a hot plate/in an oven. A 1.0 mg/ml Collagen I Standard is prepared by adding 50 μL of 2 mg/mL Type I Standard to 50 μL of 0.02 M AcOH and generating 0, 2, 4, 6, 8, and 10 μg of collagen/well. The volume is adjusted to 10μ/vial with 0.02 M AcOH. 10 μL of 12 M HCl is then added to the pressure-tight Teflon capped vial and hydrolyzed at 120° C. for 3 hrs. The vials are placed on ice, and the contents are spun down. Each vial's contents are transferred to a 96-well plate and evaporated to dryness under vacuum/on a hot plate/in an oven. 100 μL of Chloramine T reagent is added to each sample and standard and incubated at room temperature for 5 min. 100 μL of the DMAB reagent is then added to each well and incubated for 90 min. at 60° C. Absorbance is measured at 560 nm in a microplate reader. Total collagen concentration (C) is calculated as follows (C)=B/V×D μg/μL (B; amount of collagen in the sample well from Standard Curve (μg), V; sample volume added into the reaction well (μL), D: sample dilution factor).

Cough reduction Reduction of cough in subjects treated with the methods of the current disclosure can be determined by various methods. In some embodiments, the effectiveness of a dosage regimen can be determined by evaluation via a cough severity Numerical Rating Scale (NRS) test value, a Leicester Cough Questionnaire score, daytime cough frequency measured using cough count monitor device, 24-hour cough frequency measured using cough count monitor device, night-time cough frequency measured using cough count monitor device, cough quality of life questionnaire (CQLQ©) total value, Clinical Global Impression of Change (CGIC), PROMIS Item Bank v1.0-Fatigue Short Form 7a scale, St. George's Questionnaire for the IPF population (SGRQ-I) total score, EXAcerbation of Chronic pulmonary disease Tool (EXACT®) version 1.1 e-diary tool total score, Evaluating Respiratory Symptoms, (E-RS™) daily diary (the E-RS™ is an 11 respiratory symptoms item derivative instrument of the EXACT® tool) cough subscale score, chest symptoms subscale score as well as the E-RS™ total score or any combination thereof. In some embodiments, the effectiveness of a dosage regimen can be determined by evaluation via a daytime cough frequency measured using a cough count monitor device as a primary efficacy endpoint in association with secondary efficacy endpoints such as a Leicester Cough Questionnaire score.

Breathlessness reduction Reduction of breathlessness or dyspnea (including in IPF patients) in subjects treated with the methods of the current disclosure can be determined by various methods. In some embodiments, the effectiveness of a dosage regimen can be determined by evaluation via an Evaluating Respiratory Symptoms (E-RS™) breathlessness subscale score (that includes an assessment of breathlessness with activity (dyspnea)), Borg dyspnea scale value total score, Borg dyspnea scale domains (sensory-perceptual, affective distress or symptom impact), numerical rating scale dyspnea value, Modified Medical Research Council Scale, PROMIS Pool v1.0 Dyspnea Emotional Response Scale, PROMIS Item Bank v1.0 Dyspnea Severity-Short Form 10a Scale, PROMIS Item Bank v1.0 Dyspnea Characteristics Scale or any combination thereof.

Patients demonstrated a wide range of decreases in pulmonary function testing using the measures as described below that included both obstructive as well restrictive diseases in nature, as well as demonstrated by clinical testing.

Obstructive exhalation from the lungs is impeded due to airway resistance, causing a decreased flow of air.

Restrictive lung tissue and/or chest muscles fail to expand sufficiently, impeding airflow, mostly due to lower lung volumes.

Pulmonary flow testing (PFTs) include spirometry and plethysmography

Spirometry pulmonary flow from a subject is measured with a spirometer, a device with a mouthpiece designed to measure airflow.

Plethysmography pulmonary flow from a subject is measured by positioning the subject in a plethysmograph: an air-tight chamber with the appearance of a short, square telephone booth that can measure inhalation and exhalation via changes in pressure.

Measurements that can be obtained from PFT studies include the following:

Tidal volume (VT) the amount of air inhaled or exhaled during normal breathing.

Minute volume (MV) the total amount of air exhaled per minute.

Vital capacity (VC) the total air volume that can be exhaled after inhalation with maximum effort.

Functional residual capacity (FRC) the amount of air left in the lungs after exhaling normally.

Residual volume the amount of air left in the lungs after exhalation with maximum effort.

Total lung capacity the total volume of the lungs upon inhalation with maximum effort.

Forced vital capacity (FVC) the amount of air exhaled forcefully and quickly after inhalation with maximum effort.

Forced expiratory volume (FEV) the amount of air that expired during the first, second, and third seconds of the FVC test.

Forced expiratory flow (FEF) the average flow rate during the middle half of the FVC test.

Peak expiratory flow rate (PEFR) the fastest achievable rate of exhalation.

Lung diffusion capacity a test, similar to spirometry, for measuring how well oxygen moves from the patient's lungs into the patient's blood. The test can help diagnose a disease of the blood vessels between the patient's heart and lungs. It can show the amount of damage done by a disease such as emphysema, a disease in which the patient's air sacs (alveoli) are gradually destroyed.

Bronchial provocation test Triggers such as exercise, smoke, and dust can initiate labored breathing in asthmatics. A bronchial provocation test can help diagnose asthma and estimate the severity of the condition. To perform the test, the subject inhales a medication that narrows airways, after which a spirometry measurement is performed several times. Interpretation of these measurements by a doctor can indicate how airways narrow during an asthma attack.

Cardiopulmonary exercise stress test This test, generally given to subjects who may have heart disease or lung problems (which may present only during exercise), measures lung and heart strength. To perform the test, the subject walks on a treadmill or rides a stationary bicycle. During this period, both the heart rate and lung performance are monitored.

Interpretation of these measurements by a doctor can indicate the presence and/or severity of heart disease or lung problems.

Pulse oximetry test This painless test measures the oxygen content in the bloodstream. A probe is clipped to the subject's finger, earlobe, or another part of the skin. The probe uses light to measure the level of oxygen in red blood cells.

Arterial blood gas test This test measures the levels of gases such as oxygen and carbon dioxide in arterial blood. A nurse or technician withdraws blood, probably from the wrist of the subject.

Fractional exhaled nitric oxide test Certain variants of asthma present with elevated levels of nitric oxide (NO) in the body. The fractional exhaled nitric oxide test measures the NO content in exhaled air.

Body plethysmography Measures the volume of air that can be held in the lungs of a subject. The test is performed in a small, airtight room while the subject breathes against a mouthpiece.

DLCO (diffusing capacity of the lung for carbon monoxide) test Assesses how well lungs exchange gases. During the test you will inhale air containing a small amount of gas (e.g., carbon monoxide), hold your breath, then quickly breathe out. The amount of gas absorbed during the breath is measured through the gas exhaled.

Maximum inspiratory/expiratory pressures Determines respiratory muscle weakness by measuring the amount of pressure applied by the inspiratory and expiratory muscles.

Helium dilution FRC (functional residual capacity) determination This technique measures all the air in the lung that goes through gas exchange. Wearing nose clips, the subject breathes normally into a mouthpiece and then slowly exhales to empty the lungs. After repeating this a few times, you'll rest for five to ten minutes and then repeat.

Shunt Qualification

High altitude simulation testing Used for people with lung disease who are planning to travel via airplane. It helps determine if extra oxygen is needed while flying at high altitudes.

Bronchial provocation test Spirometry is used before and after inhalation of a breathing spray (e.g., methacholine) to assess the sensitivity of the airways in the lungs.

Arterial Blood Gases and Arterial Line Placement

Bronchodilator Evaluations

Sputum induction for microbiological analysis Helps create extra moisture in the airways in the lungs so patients can cough up secretions more easily. They are generally performed in a negative pressure space.

Pulmonary exercise tests allow the physician to evaluate lungs and heart under conditions of increased metabolic demand.

These decreases in pulmonary function as measured by the previous tests in different subsets of the population were also revealed in pulmonary exercise tests, including:

Six-minute walks Simple patient-paced test to assess functional capacity.

Rest and exercise test: Mild treadmill exercise at selected speeds to determine patient's oxygen needs with everyday exertion.

Incremental exercise test: Treadmill speed is increased in small increments to determine a patient's maximal exercise capacity while monitoring inspired and expired CO₂ and 02 gases.

As mentioned above in the above disclosure, SARS2 patients demonstrated both isolated changes in specific components of these PFT and pulmonary function tests and some patients across all aspects.

Patients demonstrate different patterns in changes in said measures over these time courses, including a wide range of continuous numerical values in the slope of these objective pulmonary measures described above, over time, of which some could be categorized as:
1. a relative decrease in change over time, categorized by a negative slope or a “decrease.”
2. a relative increase in change over time, categorized by a slope near zero or a “plateau.”
3. a relative increase in change over time, categorized by a slope near zero or a “decrease.”

Further, these patterns in slope could also be classified by the phase of the slopes as being generally:
1. monophasic
2. biphasic
3. triphasic
4. complex

For example, a negative slope or decrease in structural changes seen on imaging would correspond to changes in structural lung changes on imaging markedly, slowly or gradually resolving, or decreasing, or improving or lessened over time). Conversely, a negative slope or decrease in physiological or functional changes as measured using testing measures described above would gradually or markedly correspond to physiological and functional changes or markedly worsening, or pulmonary function or capacity decreasing or deteriorating over time). While these structural and physiological/functional changes generally tracked each other in an inverse manner, meaning, for example, that a decrease slope pattern in structural lung change would correlate with an increase slope pattern in pulmonary function testing or exercise testing over time (said structural and physiological/functional changes evident usually within 0.3 to 3 months of one another) and vice versa, there were a significant number (>23%) of cases in which no such relationship was observed (for a decrease slope pattern in imaging, showing improving or resolving pulmonary structural changes, however, no corresponding change, improvement, and/or increased slope pattern in PFTs or exercise testing, and vice versa for functional and imaging changes) and even, in a minority of cases (<15%) such changes were contradictory (meaning pulmonary changes for example, where there were improvements in structural changes (decrease slope pattern) but also decreased slope patterns on PFTs or exercise testing (i.e. improved structural changes but actual worsening in pulmonary functional/physiological measures, and vice versa for physiological and structural). Similarly, such findings of “consistency” and “inconsistency” were also generally observed across these phase patterns.

Further, post-SARS2 patients demonstrated these aforementioned decreases in pulmonary structure and function using said measures and further. Such changes included decreases in overall pulmonary function, pulmonary or lung reserve, and/or capacity using said tests and measures described equating to a full continuous spectrum of decreases. For example, some patients exhibited objective, or equivalent losses of <10%, 10-20%, 20-30%, 30-40%, >50%, etc. in pulmonary capacity or pulmonary reserve, and that these losses and their timing and slopes could also be described by these slope patterns and phase patterns described.

Further, counter to the current art, these changes were observed regardless of the clinical severity of their SARS2 infection. Specifically, even moderate, mild, and even clinical asymptomatic SARS2 infected patients (in addition to clinically severely infected SARS2 infected patients) all demonstrated these heretofore post-SARS2 infection pulmonary sequelae including, but not limited to the heretofore described losses in lung structure and function. Thus, all covid patients, irrespective of clinical severity, viral load, duration of viral infection or clinical symptoms, hospitalization course or absence of hospitalization, age, gender, prior comorbidities, or available treatments—all were susceptible to, and >15% of all SARS2 infected patients demonstrated these previously described losses in pulmonary function and/or structure.

TABLE 1
Summary of maximum viral RNA load per location and COVID-19 clinical
severity

| | | | |
|---|---|---|---|
| URT | LRT | Feces | Blood |

| | |
|---|---|
| Author | Maximum viral copies/mL [log 10] on the day after symptom onset |

Mild

| | | | | |
|---|---|---|---|---|
| Wölfel et al. | ~6.61 × 10⁸ | ~2.69 × 10⁸ | ~3.55 × 10⁷ | ND |
| [7] a | on Day 4 | on Day 6 | on Day 9 | |
| | (6.66 × 10⁸ | (7.11 × 10⁸ | | |
| | in publ.) | copies/swab in publ.) | | |
| Zou [13] # | ~2.19 × 10 7 | ND | ND | ND |
| | on Day 4 | | | |
| G. Lui et al. | 2.50 × 10⁶ | ND | 7.94 × 10³ | ND |
| [20],* | on Day 4 | | on Day 7 | |
| Zheng et | ND | ~2.00 × 10⁷ | ND | ND |
| al. [12], a | | on Day 11 | | |

Moderate-Severe

| | | | | |
|---|---|---|---|---|
| Wölfel et al. | ND | ND | ND | ND |
| [7] a | | | | |
| Zou [13] a | ~1.32 × 10⁸ | ND | ND | ND |
| | on Day 5 | | | |
| G. Lui et al. | 4.60 × 10⁹ | 3.45 × 10⁸ | 2.76 × 10⁶ | 1 × 10⁴ |
| [20],* | on Day 8 | on Day 11 | on Day 18 | on Day 3 |
| Zheng et al. | ND | ~1.82 × 10⁶ | ND | ND |
| [12] a | | on Day 4 | | |

LRT: Lower respiratory tract, ND: Not determined, URT: Upper respiratory tract;
*all subjects in this study received Lopinavir/Ritonavir; publ.: Publication; an estimated data as digitalized from the graph. Number in brackets refer to listing in the reference list at Table 4 of Weiss, A. et al., Spatial and temporal dynamics of SARS-CoV-2 in COVID-19 patients: A systematic review and meta-analysis, 58 EBIOMEDICINE 102916 (2020).

### Example 1

Inhaled corticosteroid and Beta2 agonist therapy during the acute phase transition from primary viral-mediated pneumocyte and lung parenchymal damage phase to induced innate and/or adaptive immune-mediated pneumocyte and lung parenchymal damage phase mitigates loss in lung function and pulmonary structure due to SARS2. This transition period can be determined in any number of ways, including but not limited to laboratory, blood, serum, urine, fecal, sputum, bronchial lavage, imaging, genomic, or tissue-based testing. This transition typically occurs clinically around 6-14 days from symptom onset, and in some instances can be demarcated in shifts to elevated levels of, or an increased slope in certain blood factors such as LDH, ferritin, complement, and C-reactive protein, or increasing degree or distribution of lung opacities consolidation, edema, lymphatic prominence, interstitial thickening, pulmonary emboli, pulmonary hypertension, large and medium-size vessel wall thickening or inflammation, and cardiac inflammation or enlargement, or heart failure. During this period, pharmacologic agents and combinations as disclosed herein can be administered as a means to limit, contain, or restrain inflammatory/immune mediated damage and limit the progression of SARS2 associated lung damage as described above both physiologically functionally and structurally. This drug can be administered in asymptomatic, mild, moderate, and severe SARS2 disease patients. Ideally, within 4-7 days of this transition from primary viral-mediated lung damage to immune-mediated (the acute phase of the infection).

A patient with mild clinical Covid symptoms was treated early during the acute phase of infection after viral PCR SARS-CoV-2 infection confirmation, with Symbicort twice a day—an inhaled corticosteroid (budesonide 160) and long-acting beta2 agonist (formoterol 4.5) from the initial SARS2 rt-PCR positive test, confirming their SARS2 infection. This patient was treated for 2 weeks during the clinical period before and upon discharge as continuously as maintenance for several weeks, after which the medication was then discontinued.

A similar patient but with moderate to severe disease (with markedly elevated CRP LDH and ferritin levels relative to hospital standard reference values) was treated later during the course of their infection after peak clinical severity with Advair 100/50 twice a day—an inhaled corticosteroid (fluticasone) and beta2 agonist (salmeterol) in addition to supportive medications and treatments. This patient was treated during their clinical period and then maintained on this therapy for 6 weeks afterward and then tapered down to an as-needed basis. At 3 and 4 months, respectively, both patients have reported and were noted to have significantly decreased loss in lung function by PFTs relative to others that had not had similar treatments and no evidence of lasting structural change by radiological imaging. In contrast, similar patients, as well as asymptomatic patients, demonstrated volume loss and mild to moderate lung fibrosis, loss in capacity and reserve of 20-30%, and increased exertional dyspnea, fatigue, and apparent hyperreactive airways.

### Example 2

Inhaled corticosteroid and Beta2 adrenoreceptor agonist therapy maintain lung function and structure during the post-acute phase, improves lung capacity and exertional tolerance, and mitigates or prevents progressive loss of lung function and pulmonary structure over time due to SARS2. This transition period can be determined in any number of ways, including but not limited to laboratory, blood serum, urine, fecal, sputum, imaging, bronchial lavage, genomic, or tissue-based testing. This transition, typically, though not exclusively, occurs clinically when peak structural findings on imaging (example, chest Xray, CT scan, or MRI) have reduced by around 50% or more from peak severity and/or when laboratory values have trended downwards for 2 successive days and/or are now near or below the upper bounds of normal for ferritin, LDH and CRP, and/or when oxygen requirements and support have diminished by 50% or greater from peak severity/requirement. During this period, pharmacologic agents and combinations as disclosed herein can be administered to limit, contain, improve or restrain inflammatory/immune mediated damage and limit the progression of SARS2 associated lung damage as described above both physiologically, functionally, and structurally. This can be administered in asymptomatic, mild, moderate, and severe SARS2 disease patients.

A patient with mild clinical COVID symptoms was treated at the end of the first week from time of confirmed COVID diagnosis, with Symbicort twice a day—an inhaled corticosteroid (budesonide 160) and long-acting beta2 agonist (formoterol 4.5). This patient was treated for 2 weeks during the clinical period before and upon discharge, after which the medication was then discontinued.

A similar patient was treated on day 10 with Advair 100/50 twice a day—an inhaled corticosteroid (fluticasone) and beta2 agonist (salmeterol). This patient was treated during their clinical period and then maintained on this therapy for 2 weeks afterward and then tapered down to an as-needed basis. At 3 and 4 months, respectively, both patients have reported and were noted to have significantly decreased loss in lung function by PFTs (relative to similar patients that were not treated in this manner) and no evidence of lasting structural change by radiological imaging. In contrast, similar patients, as well as some asymptomatic patients, who were not similarly treated, demonstrated volume loss and mild to moderate lung fibrosis, loss in capacity and reserve of 20-30%, and increased exertional dyspnea and hyperreactive airways.

### Example 3

Pirfenidone and Nintedanib therapy during the post-acute phase mitigates functional and structural lung loss, preserves lung function and structure, and can improve lung capacity and exertional tolerance in patients previously infected SARS2 with significant pulmonary functional and structural loss as a result of their severe infections. This transition period can be determined in any number of ways including but not limited to laboratory, blood serum, urine, fecal, sputum, bronchial lavage, imaging, genomic or tissue-based testing. These patients can also be characterized in one embodiment as those patients with prolonged hospitalization due to lung disease, prolonged ventilation, course of ECMO, high peaks during hospitalization 1.5 times above the upper of normal for LDH, or Ferritin, or CRP, >30% loss in pulmonary capacity or pulmonary reserve, significant scarring and/or fibrosis seen by CT imaging, MRI, PET imaging or chest x-ray, significant reductions in physiological and functional measures as assessed by the objective physiologic and functional assessments described above, and/or a progressive or worsening pattern of any of such measures over time. During this period, pharmacologic agents and combinations as disclosed herein can be administered to limit, contain, or restrain or reduce such damage and limit the progression of severe SARS2 associated lung damage as described above both physiologically functionally and structurally. A patient with severe clinical COVID symptoms and severe lung damage, and >40% loss of pulmonary reserve by objective testing was treated with pirfenidone starting 1 month after discharge. At 3 months, an improvement in PFTs and reduction in progression of structural lung damage by high-resolution CT (HRCT) imaging was observed. A similar patient was treated with a 50% loss of pulmonary reserve. Two months post-discharge showed slight structural damage and physiologic testing, and the patient was initiated on nintedanib. At 4 months, both the structural and physiologic declines appeared stabilized and arrested.

### Example 4

Ramakrishnan et al., Inhaled budesonide in the treatment of early COVID-19 illness: a randomized controlled trial, MEDRXIV PREPRINT, https://doi.org/10.1101/2021.02.04.21251134, last updated Feb. 8, 2021, reported that administrating 800 μg inhaled budesonide twice a day in adults aged 19-79 within seven days of the onset of mild COVID symptoms reduced the likelihood of needing urgent medical care and reduced recovery time.

Budesonide, the active component of PULMICORT TURBUHALER (budesonide) 200 μg, is a corticosteroid designated chemically as (RS)-11β,16α,17,21-tetrahydroxypregna-1,4-diene-3,20-dione cyclic 16,17-acetal with butyraldehyde. Budesonide is provided as a mixture of two epimers (22R and 22S). Budesonide is indicated for the maintenance treatment of asthma as prophylactic therapy in adult and pediatric patients six years of age or older. It is also indicated for patients requiring oral corticosteroid therapy for asthma. The starting dose for adults is 200-400 μg BID and going up to 400 μg BID, or starting at 400-800 μg BID and going up to 800 μg BID. The numbers are lower for children, who start at 200 μg or 400 μg BID and are recommended to go up to 400 μg BID. See Pulmocort Turbuhaler, https://www.rxlist.com/pulmicort-turbuhaler-drug.htm, (last viewed Mar. 4, 2021).

Patient symptoms were measured through self-reported evaluations, such as the Common Cold Questionnaire (CCQ), a metric developed by the Hunter Medical Research Institute which records general, nasal, throat, and chest cold-like symptoms and scores them based on severity, and the InFLUenza Patient-Reported Outcome (FluPRO®), a 32-item instrument developed by Evidera® that assesses the severity of influenza-like symptoms across the body. Additionally, patient conditions were assessed directly through pulse oximeter readings, body temperatures, and SARS-CoV-2 viral loads obtained with nasopharyngeal swabs by trained clinical staff.

In a study of 70 patients treated with inhaled budesonide only (BUD), one participant required medical intervention. In the control group of 69 patients receiving routine clinical treatments (“usual care,” or UC), ten participants required intervention. Patients in the BUD arm self-reported clinical recovery one day quicker than patients in the UC arm. On day 14 of treatment, self-reported symptoms were present in 10% of BUD participants than 30% of UC participants.

The primary outcome occurred in ten participants in the UC arm versus one participant in BUD arm (difference in proportions 0.131, 95% CI (0.043, 0.218), p=0.004), indicating a relative risk reduction of 90% for BUD. Self-reported clinical recovery was 1 day quicker with BUD than UC (median of 7 days versus 8 days, logrank test p=0.007). The mean time to recovery in days was 8 and 11 in the BUD and UC arm, respectively. At day 14, self-reported symptoms were present in 10% (n=7) of participants randomized to BUD compared to 30% (n=21) of participants randomized to UC (difference in proportion 0.204, p=0.003). Symptom resolution at day 14, as defined by the FLUPro® user manual, occurred in 82% and 72% of the BUD and UC arms, respectively (p=0.166). The median time to symptom resolution measured by the FLUPro® was 3 and 4 days in the BUD and UC arms, respectively (logrank test p=0.080). The mean change (95% CI) in FLUPro® total score between day 0 and day 14 in the BUD and UC are −0.65 (−0.80 to −0.50) and −0.54 (−0.69 to −0.40) respectively (mean difference of −0.10, 95% CI of the difference −0.21 to −0.00, p=0.044). FLUPro® domains showed that systemic symptoms were significantly greater in BUD than UC (supplementary table 3). The mean change in CCQ total score between day 0 and day 14 in the BUD and UC was −0.49 (−0.63 to −0.35) and −0.37 (−0.51, −0.24) respectively (mean difference of −0.12 (−0.21 to −0.02), p=0.016). In a ‘virtual twin’ study design, stochastic simulations demonstrated that the daily odds ratio reached the primary outcome, with BUD reduced by 3000%.

As can be seen, inhaled budesonide reduced the likelihood of requiring urgent care, emergency department consultation, or hospitalization. The inhaled glucocorticoid, budesonide, given for a short duration, may effectively treat early COVID-19 disease in adults. With a relative reduction of 90% of clinical deterioration, this effect is equivalent to the efficacy seen following COVID-19 vaccination. The broad inclusion criteria make this study intervention relevant to health care systems worldwide. A quicker resolution of fever, a known poor prognostic marker in COVID-19, and a faster self-reported and questionnaire reported symptom resolution. Fewer participants had persistent COVID-19 symptoms at 14 and 28 days after budesonide therapy compared to usual care.

Patients treated with budesonide had the less severe disease by self-measurement, 90% reduction in the need for a hospital visit, reduced symptoms, such as fever, fatigue, shortness of breath at 14 and 28 days. This study provides direct clinical support in actual patients for the benefits of inhaled steroids to reduce COVID-induced disease severity and/or sequelae.

### Example 5

Arnold et al., Patient outcomes after hospitalization with COVID-19 and implications for follow-up: results from a prospective UK cohort, 0 THORAX 1-4 (2020) reported a study of adults aged 18-94 with laboratory-confirmed SARS-CoV-2 infections found that a significant number of individuals experienced persistent symptoms and a decline in health-related quality of life (HRQoL) several months after the initial onset of disease. The symptoms surveyed included fatigue, loss of taste or smell (anosmia), head and body aches, difficulty breathing, sore throat, cough, runny nose, diarrhea, sweats, rash, chills, fevers, and nausea. A total of 177 individuals participated in the survey. Eleven were asymptomatic, 150 experienced mild illness, and 16 had moderate or severe disease requiring hospitalization during the initial infection phase. A group of 21 healthy individuals was simultaneously surveyed to act as a control.

Patients were followed-up with a median of 83 days (IQR 74-88 days) after hospital admission and 90 days (IQR 80-97 days) after COVID-19 symptom onset. Eighty-one (74%) patients reported at least one ongoing symptom: 39% breathlessness, 39% fatigue, and 24% insomnia (see FIG. 1). Sixteen (59%) patients in the mild COVID-19 group reported ongoing symptoms compared with 49 (75%) and 16 (89%) in the moderate and severe group, respectively.

Of the 15/110 (14%) patients with abnormal follow-up radiographs (n=10 moderate group, n=5 severe group), two worsened from hospital admission with higher radiographic severity scores (both had known previous interstitial lung disease). Findings seen included consolidation (one patient), reticulation (eight patients), atelectasis (collapsed lung, five patients), and pleural effusion (one patient). Nine patients showed fibrotic changes in two patients with moderate disease at baseline (other HRCT results: normal (four), minor persistent ground glass changes (two), pleural effusion (one)). Eleven patients had restrictive spirometry, and fifteen had a significant desaturation on the STS test, all within the severe or moderate group (see the online supplementary material). SF-36 scores demonstrated a reduction in reported health status across all domains compared with age-matched population norms.

The follow-up survey was completed in a median range of 169 days (31-300 days) after the initial onset. Persistent symptoms were reported by about 30% of individuals. Patients aged 65 and older reported persistent symptoms at a slightly higher rate (43.3%). The most common symptoms were fatigue (13.6%) and anosmia (13.6%). A total of 51 outpatients and hospitalized patients (30.7%) reported worse HRQoL than baseline versus four healthy/asymptomatic patients (12.5%). These findings support that there is a persistent sequalae after COVID-19 infection, even in mild and asymptomatic cases, as far out as 6 months compared to healthy controls.

### Example 6

Logue et al., Sequelae in Adults at 6 Months After COVID-19 Infection, 4 JAMA NETWORK OPEN 1-4 (2021), available at https://jamanetwork.com/journals/jamanetworkopen/fullarticle/2776560 reported a study of adults aged 32-71 in the UK with laboratory-confirmed SARS-CoV-2 infections which required hospitalization found that a significant proportion remained symptomatic 3 months following diagnosis. Patients were recruited at diagnosis and had their baseline demographics, comorbidities, and blood test results recorded. Follow-up was performed at a respiratory outpatient clinic 8-12 weeks after hospital admission. Chest radiographs, spirometry, exercise testing, blood tests, and quality-of-life and mental-wellbeing questionnaires were administered to determine the severity and nature of any abnormalities. Patients were subdivided into groups based on the severity of their disease (mild, moderate, or severe).

Out of 110 patients, 81 (74%) reported at least one ongoing symptom. The most common were fatigue (39%), breathlessness (39%), and insomnia (24%). The likelihood of ongoing symptoms increased proportionally to the severity of illness. Fifteen patients presented with abnormal radiographs on follow-up, with two worsening their initial condition at hospitalization. Eleven patients had restrictive spirometry, and fifteen showed significant desaturation upon exercise testing, all from within the moderate and severe illness groups. Blood testing revealed that 35 patients with significantly deranged liver and renal functions at admission had mostly (91%) returned to baseline at follow-up. Health-related quality-of-life (HRQoL) scores were reduced in all domains compared with age-matched population norms.

### Example 7

Huang et al., 6-month consequences of COVID-19 in patients discharged from hospital: a cohort study, 367 THE LANCET 220-232 (2021) reported a cohort study of adults aged 47-65 in Wuhan, Hubei, China with laboratory-confirmed SARS-CoV-2 infections which required hospitalization found that a significant number of patients displayed ongoing symptoms six months after initial onset. Patients were recruited from those discharged from Jin Yin-tan Hospital between Jan. 7, 2020, and May 29, 2020. Follow-up visits were performed at the outpatient clinic of Jin Yin-tan Hospital.

All patients were interviewed with a series of questionnaires to evaluate symptoms and HRQoL, underwent physical examinations and a walking test and received blood tests. Additionally, a stratified sampling procedure was used to select patients based on the severity of their disease to undergo pulmonary function tests, ultrasounds, and high-resolution chest computerized tomography (CT) scans. Patients who were enrolled in the Lopinavir Trial for Suppression of SARS-CoV-2 in China (LOTUS China) tested the effects of lopinavir-ritonavir treatment on clinical recovery time received SARS-CoV-2 antibody tests.

In total, 1733 patients were enrolled in the study, 76% reported at least one symptom at follow-up. Fatigue or muscle weakness (63%), sleep difficulties (26%), and hair loss (22%) were the most common reported symptoms. Anxiety or depression was reported among 23% of patients. Six-minute walking test showed that 23% of patients presented with a shorter walked distance than the lower limit of the normal range for their age group. Pulmonary function tests showed that patients with the most severe disease (those requiring high-flow nasal cannulas or ventilation) displayed a much higher rate of diffusion impairment (56%) than those who did not (27%). Chest CT scans revealed that about half (53%) of all patients displayed at least one abnormal CT pattern, with overall CT scores increasing proportionally with the severity of the disease. Of the 94 patients enrolled in the LOTUS China trial, the seropositivity and median titers of their neutralizing blood antibodies were significantly lower at follow-up (58.5% and 10.0, respectively) than they were during the acute phase (96.2% and 19.0, respectively).

A considerable proportion of participants (22-56% across different severity scales) had a pulmonary diffusion abnormality 6 months after symptom onset. This finding was consistent with the most common abnormal CT pattern was a pulmonary interstitial change (GGOs and irregular lines), which were similar to the long-term lung manifestations of SARS27 or influenza. However, the results from the Huang study did not suggest that corticosteroids could accelerate the recovery of lung injury based on the pulmonary function assessment or chest imaging.

### Example 8

A study of adults with a median age of 49 years (IQR 27 years) with COVID-19 from Hong Kong found that over half of patients with asymptomatic or mild cases demonstrated chest radiograph (CXR) abnormalities during active infection. These findings were also directly correlated with baseline reverse transcriptase-polymerase chain reaction (RT-PCR) SARS-CoV-2 testing at diagnosis. As measured by RT-PCR cycle threshold values, patients with higher viral loads were at significantly greater risk for these sequelae at 3 months post viral recovery.

Study participants were RT-PCR positive patients who recovered from SARS-CoV-2 infection between February and August 2020. These patients had clinical and CXR evaluations at initial diagnosis, during isolation with the active infection, and at ≥3 months after recovery and hospital discharge. The 3-month follow-up consisted of an outpatient clinic visit with face-to-face evaluation by a healthcare professional completed ≥3 months after recovery and discharge. Specific symptoms reported at this follow-up visit and diagnosis and upon recovery were recorded. CXRs performed in either posteroanterior or anteroposterior projections within 24 hours of the initial positive RT-PCR test, during an active infection, and at 3-month outpatient, follow-up was used for analyses.

COVID-19 clinical severity was assessed at initial presentation using an ordinal scale recommended by the World Health Organization's (WHO) R&D Blueprint expert group. The ordinal scale categories are: 0=no clinical or virological evidence of infection; 1=no limitations of activities; 2=limitation of activities; 3=hospitalized but not requiring oxygen therapy; 4=hospitalized and requiring oxygen by mask or nasal prongs; 5=requiring non-invasive ventilation or use of high-flow oxygen; 6=requiring intubation and mechanical ventilation; 7=requiring ventilation and additional organ support; and 8=death. For this study, a score of ≤3 was defined as a mild or asymptomatic disease. The 3-month follow-up consisted of an outpatient clinic visit with face-to-face evaluation by a healthcare professional completed ≥3 months after recovery and discharge. Specific symptoms reported at this follow-up visit and at diagnosis and upon recovery were recorded.

In total, 168 patients fulfilled the study criteria. Twenty-two patients (13.1%) were asymptomatic at presentation, of which 20 remained asymptomatic throughout their entire course of viral infection. The most common symptoms at diagnosis were respiratory symptoms (58.9%) for symptomatic patients, such as cough and fever (42.3%). At the 3-month follow-up, 22 (13.1%) patients had persistent symptoms, with respiratory symptoms being the most common (45.5%). Of these 22 patients, 15 (68%) had RT-PCR cycle threshold values <25 at initial diagnosis, indicating a high initial viral load.

During the active infection phase, 85 (50.6%) of patients eventually presented with abnormal CXRs. At the 3-month follow-up, 20 (11.9%) patients had persistent abnormal CXR findings. Three of the 22 (13.6%) asymptomatic patients at diagnosis had persistent CXR abnormalities at 3 months, accounting for 3 (15.0%) out of the 20 patients with abnormal 3-month follow-up CXRs. All 20 of the patients with abnormal 3-month CXRs also had abnormal CXRs during the active infection phase.

In summary, about 12% of all patients had persistent abnormalities and symptoms three months after initial recovery from the viral infection. About 50% of mild and asymptomatic COVID-19 patients had abnormal CXRs during active infection. Twenty-five percent of the mild and asymptomatic patients with an abnormal CXR at diagnosis were found to have persistent CXR abnormalities at 3-month follow-up. An RT-PCR cycle threshold value <25 and a maximum CXR severity score of greater the one predicted the symptoms and CXR abnormalities at 3-month follow-up, respectively.

No correlation was identified between initial RT-PCR cycle threshold values and CXR findings at the 3-month follow-up. Indeed, only two of the twelve mild and asymptomatic COVID-19 patients treated with steroids during active infection went on to have persistent symptoms three months after the initial viral recovery. Similarly, only three of the twelve patients with moderate or severe infection who were treated with steroids went on to have persistent abnormalities, suggesting that the lungs were damaged in both patient groups, as shown in the chest imaging at three months post viral.

Post COVID-19 patients did not express asthma or post-viral reactive airways. None of the preceding examples described their findings with the typical words or phrases consistent with asthma-fatigue, exertional dyspnea, shortness of breath, hyperreactive airways, bronchospasm, or other physical descriptions of a patient with asthma or COPD. Thus, based on the patients' symptoms alone, a physician would not have considered using inhaled steroids to treat the symptoms of the virus during infection or after the initial recovery.

It is also generally known and well-accepted that one should not dose patients with inhaled steroids who did not have asthma or COPD. A prominent reason is the side effects of chronic use. For example, Hanania, N. et al., Adverse Effects of Inhaled Corticosteroids, 98 AM. J. MED. 196-208 (1995) states that while inhaled steroids can treat asthma, they should be minimized in use and dose because of the side effects, including adrenal suppression, bone loss, skin thinning, increased cataract formation, decreased linear growth in children, metabolic changes, and behavioral abnormalities.

Another reason is the inability of inhaled steroids to effectively treat acute viral respiratory tract infections, even in a patient with asthma. Doull, I J M et al., Effect of inhaled corticosteroids on episodes of wheezing associated with viral infection in school age children: randomised double blind placebo controlled trial, 315 BMJ doi: https://doi.org/10.1136/bmj.315.7112.858 (last updated Oct. 4, 1997) reports a study that shows no clear benefit for inhaled steroids in kids with asthma and viral infection for reducing virus-associated wheezing. Systemic corticosteroids decreased the illness associated with such wheezing in adults, but in younger children, inhaled corticosteroids offered minimal benefits, either when given at the onset of symptoms or continuously. Thus, before learning the treatment methods disclosed herein, a person having skill in the art would not have treated a subject for a pulmonary disease or symptom, which is the consequence of previous exposure of the subject to SARS-CoV-2 by administering to the subject of an effective amount of one or more corticosteroids, such as inhaled budesonide.

### Example 9

A hypertensive 43-year-old female patient (BMI=44.1) was exposed to and tested positive for COVID in September 2021. The patient was previously vaccinated with both doses of the Pfizer/BioNTech vaccine in February 2021. The patient takes 25 mg lisinopril/12.5 mg hydrochlorothiazide once daily for blood pressure maintenance and 1000 mg metformin twice daily for insulin resistance.

Following infection, the patient developed fever, fatigue, shortness of breath, and persistent cough. One spray of fluticasone propionate (50 μg per metered dose) was administered nasally to each nostril, with the primary intent to both treat ongoing acute symptoms and to prevent the development of post-acute covid pulmonary sequalae. Treatment resulted in a subsidence of cough within about 5 minutes for 1-2 hours. Administration was repeated up to eight times daily per nostril during the acute phase. Four weeks after disease onset and following initial recovery, the patient's acute phase pulmonary symptoms significantly subsided, to only intermittent cough in the immediate post-acute phase. Administration was reduced to one spray per nostril twice daily. As in the acute phase, the cough subsided within about 5 minutes but for a longer time than in the acute phase, lasting 3-4 hours. Within 4 weeks, cough had mostly resolved. No other post-acute pulmonary sequelae presented or developed. The patient's pulmonary status had returned to pre-COVID baseline status.

### Example 10

Cho et al. reported a 100-patient prospective clinical study in “Small airways disease is a post-acute sequela of SARS-CoV-2 infection,” MedRxiv May 30,2021, incorporated herein by reference in its entirety. Covid-19 patients who remained symptomatic more than 30 days following initial diagnosis (post-acute pulmonary sequelae) were consistent with functional small airway disease (fSAD), including air trapping on imaging, clinically, and by pulmonary function tests. Further, this fSAD was independent of infection severity.

Acute COVID-19 infection in Cho was the twenty-one days following diagnosis. Patients were asked to retrospectively recount their symptoms during the acute phase of infection and detail whether these symptoms persisted using a structured questionnaire. The modified Medical Research Council (mMRC) dyspnea scale quantified breathlessness, where 0 indicates dyspnea only with strenuous exercise and 4 indicates dyspnea when dressing. Laboratory testing, pulmonary function testing, and chest imaging on their clinic visit were part of the clinical protocol. Pulmonary function testing and chest CT data were then a cohort of healthy non-smoking control subjects.

Patients and healthy subjects underwent non-contrast chest CT with an inspiratory scan coached to total lung capacity (TLC) and an expiratory scan coached to residual volume (RV). Texture analysis to quantify ground-glass opacities (GGO) as a percent of total lung volume at TLC by using grayscale patterns within CT images was performed. DPM (disease probability measure (VIDA diagnostics) was also performed, which quantifies the voxel-to-voxel difference in Hounsfield Units (HU) between matched inspiratory and expiratory images to estimate the probability of air trapping such that the probability is inversely proportional to the relative differences in HU19. When quantified using DPM analysis, air trapping reflects functional small airways disease (fSAD).

The median age of the patients in their study was 48 years (IQR, 36.3 to 60.5 years), and 66% were female. Patients hospitalized or in the ICU were significantly older than ambulatory patients (P<0.001 and P<0.01, respectively). At least one co-existing illness was present in 76 patients. Obesity (59%) and hypertension (27%) were the most common co-existing illnesses. Critically ill patients were more likely to have chronic kidney disease, chronic obstructive pulmonary disease (COPD), type 2 diabetes mellitus, and hypertension than ambulatory patients. The most common co-existing pulmonary disorder was asthma (26%), followed by COPD (6%) and interstitial lung disease (4%).

Most patients were never smokers (75%). Smoking was more common among hospitalized or ICU patients than ambulatory patients. Among former and current smokers, median pack-years were 8 (IQR, 4.8-25.5). The median time to follow-up in the post-COVID ambulatory clinic was 74.5 days (IQR, 45.8-118). The most common symptoms during acute COVID-19 infection were fatigue (83.7%), dyspnea (82.3%), and cough (71.4%). The median length of admission was four days (IQR, 2.5-7) for the hospitalized group and 18.5 days (IQR, 10.8-42.3) for the ICU group (P<0.001). None of the ambulatory patients required supplemental oxygen during acute illness. Ten (58.8%) of the hospitalized patients and all the critically ill patients required supplemental oxygen. The median maximal requirement of hospitalized patients requiring supplemental oxygen was 2 liters per minute (IQR, 0-2). Of the ICU patients, 14 (87.5%) required high-flow oxygen, 11 (68.8%) were mechanically ventilated, and 3 (18.8%) required extracorporeal membrane oxygenation (ECMO). The median maximal fraction of inspired oxygen for ICU patients was 1.0 (IQR, 0.65-1.0). The most commonly reported persistent symptoms at follow-up were dyspnea (73%), fatigue (56%), and cough (34%).

The median score on the mMRC dyspnea scale at follow-up was 2 (IQR, 0-2). Compared to the ambulatory group (38.8%), a higher proportion of patients in the hospitalized (70.6%) and ICU (75%) groups had an mMRC score greater than or equal to 2 (P<0.01). The median percent predicted pre-bronchodilator forced vital capacity (FVC) and forced expiratory volume in one second (FEV1) in post-acute COVID-19 patients were 95% (IQR 81-106) and 93% (IQR 78-104), respectively. Compared to the ambulatory group, the hospitalized and ICU groups had a lower FVC (P<0.01 and P<0.001, respectively) and FEV1 (P<0.05 and P<0.001, respectively). The FVC and FEV1 in ambulatory patients were not different than healthy controls. The median pre-bronchodilator FEV1/FVC was 0.8 (IQR, 0.76-0.84), and there were no differences across groups. No clinically significant response to bronchodilator was observed for FVC or FEV1 in any of the PASC groups.

The median percent predicted total lung capacity (TLC) was 96% (IQR, 83.5-109), and median residual volume was 82% (IQR, 64.3-98.8) in PASC patients. Compared to ambulatory patients, hospitalized and critically ill patients had lower TLC (P<0.01 and P<0.001, respectively). Critically ill patients also had lower RV than ambulatory patients (P<0.01). TLC and RV were similar between the ambulatory group and healthy controls. The median percent predicted diffusing capacity for carbon monoxide (DLCO) was 96% (IQR, 79-111.5) in PASC patients. Hospitalized and critically ill patients had a significantly lower DLCO than ambulatory patients (P<0.001 for both). Similar differences in spirometry, lung volumes, and DLCO were found in our age- and BMI-adjusted multivariate analysis.

Images from inspiratory and expiratory chest CT were available for analysis for 91 patients (91%). The most common abnormalities identified by the qualitative analysis were air trapping (58%), GGO (51%), and pulmonary nodules (35%). Hospitalized and critically ill patients were more likely to have bronchiectasis and architectural distortion, honeycombing, or scar than ambulatory patients. To more fully assess GGO and air trapping, a quantitative analysis of chest CT images was performed against healthy controls. Among hospitalized and ICU patients, the mean percent of total lung classified as GGO was 13.2% and 28.7%, respectively, and was higher than in ambulatory patients (3.7%, P<0.001 for both comparisons). Although the GGO observed in the ambulatory group was low, it was higher than healthy controls (0.06%; P<0.001). GGO as a percentage of total lung correlated with the percent predicted TLC (p=−0.6; P<0.001) and DLCO (p=−0.47; P<0.001). DPM analysis revealed that a substantial percentage of the total lung was affected by air trapping in all severity groups.

The amount of air trapping across PASC groups did not differ. The mean percentage of total lung affected by air trapping was 25.4%, 34.5%, and 27.2% in the ambulatory, hospitalized, and ICU groups and was higher in ambulatory patients than in healthy controls (7.3%; P<0.001). These differences persisted after adjusting for age and BMI. Air trapping can also be detected using lung volume measurements, specifically the RV to TLC ratio (RV/TLC). The RV/TLC ratio was higher in the hospitalized group than the ambulatory group (P<0.01) by plethysmography but did not differ across groups when measured by quantitative CT. RV/TLC calculated using plethysmography correlated with the presence of air trapping measured by DPM (p=0.6; P<0.001) as did RV/TLC calculated using quantitative CT (p=0.84; P<0.001). Hospitalized and ICU patients were more dyspneic than ambulatory patients as measured by the mMRC score, but the frequency of dyspnea and cough did not differ across severity groups. PASC patients who required hospitalization or ICU care during acute infection had restrictive physiology and impaired gas exchange on pulmonary function testing, characterized by a reduction in FVC, FEV1, TLC, and DLCO. On chest CT imaging, these patients were more likely to have bronchiectasis and architectural distortion, honeycombing or scar, and bronchiectasis than the ambulatory group.

In contrast to most published reports of post-acute COVID-19, most PASC patients in their cohort had mild disease during acute infection. Spirometry and lung volumes were normal in these patients and differed from a cohort of healthy control subjects. They observed a higher DLCO in the ambulatory group than healthy controls. Increases in DLCO can be driven by pulmonary capillary blood volume, as occurs in asthma and obesity. About a quarter of patients in the ambulatory group had asthma. The BMI of the ambulatory group was significantly higher than healthy controls in the presence of GGO and air trapping in ambulatory patients. Than hospitalized and critically ill patients, fewer patients in the ambulatory group had GGO on qualitative chest CT images, and a smaller percentage of the total lung was classified as GGO by texture analysis.

Texture analysis revealed that GGO as a percentage of the total lung was significantly higher in the ambulatory group than healthy controls, suggesting ongoing lung inflammation, edema, or fibrosis in PASC patients. A striking proportion of patients in all three PASC groups had air trapping on qualitative CT analysis. The percent of total lung classified as air trapped using DPM analysis did not differ across PASC groups. DPM measures significantly more air trapping in the ambulatory group than healthy controls. Air trapping occurs because of partial or complete airway obstruction in regions of the lung. They did not observe airflow obstruction by spirometry in any group, suggesting that air trapping in their cohort is due to small rather than large airways involvement.

Small airways, defined as non-cartilaginous airways with an internal diameter <2 mm, contribute little to total airway resistance. Thus, spirometry does not detect small airway disease until a large percentage (>75%) of all small airways are obstructed. While lung imaging does not readily identify small airways, studies have established air trapping on chest CT as a biomarker of functional small airways disease (fSAD). Taken together, their findings suggest that SARS-CoV-2 infection itself leads to fSAD and air trapping. Restrictive lung disease and impairment in gas exchange results from lung injury and ARDS, regardless of the underlying cause. The fSAD observed in PASC patients (PASC-fSAD) could result from direct infection of the small airways by SARS-CoV-2, even in patients with mild acute infection. In this case, PASC-fSAD may result from an ongoing injury-repair process, cellular debris, and/or abnormal mucus production. They did not observe a correlation between dyspnea and the severity of either GGO or air trapping measured by quantitative CT analysis. In addition to fSAD, other pathological processes may contribute to respiratory symptoms.

Based on Cho et al., PASC/Long Covid is most visibly a small airways disease. Beta 2 agonists do not seem to affect it much, at least from a physiological perspective. Either the B2 is not getting there, or that it is mostly inflammatory, or both. Smaller particle size of the drug (extrafine) is needed to get peripheral small airways disease distribution and, to some degree, the delivery device distributing those particles. Dosing of one or more corticosteroids can begin with current particle sizes, for example, formoterol fumarate dihydrate inhaler dosage (Symbicort is 4.5 μg) with beclomethasone dipropionate (QVAR is 40, 80, 150, or 320 μg). Then the dosing can be adjusted for extrafine particles (<2.1 μg MMAD) and/or geometric standard deviation (GSD) (<1.2 μg).

### Example 11

Numerous studies have shown that managing small airways disease depends on accurately delivering the drug to the small airways. These factors can be mitigated principally by adjusting particle and/or dose size, and distribution, generally favoring smaller or “extrafine” particles (defined <2.1 μg MMAD) and/or geometric standard deviation (GSD)<1.2 μg), and delivery devices that patients can readily and easily draw the drug cloud in relatively uniform distribution to the small airways and not to the mouth, oropharynx, trachea, large airways, or other undesired locations. Further, this dosing method can improve outcomes and control of small airways disease and symptoms and processes, including small airways obstruction, inflammation, hyperreactivity, bronchospasm, cough, mucus production, and the typical pulmonary/respiratory symptoms for asthma and COPD.

Further, lower doses (typically on the order of 1.5-2.5 fold reductions from standard non-extrafine formulations) and decreased side effects for the drugs can be used. For example, FOSTAIR Nexhaler (a next-generation multidose inhalation device) containing extrafine beclomethasone dipropionate 100 μg and formoterol fumarate dihydrate 6 μg has shown improved efficacy over non-extrafine formulations and standard delivery. Similarly, glycopyrronium/formoterol fumarate dihydrate 14.4/10 μg (equivalent to glycopyrrolate/formoterol fumarate 18/9.6 μg) delivered by pressurized metered-dose inhaler (pMDI) using novel co-suspension delivery technology. The co-suspension delivery technology uses a strong nonspecific association between micronized drug crystals and phospholipid-based porous particles when suspended together in the pMDI propellant hydrofluoroalkane (HFA) 134a (1,1,1,2-tetrafluoroethane), resulting in uniform suspensions and consistent dose delivery.

Similarly, high-dose regimens and long-term use of inhaled corticosteroids (ICSs) can cause side effects similar to those observed with systemic corticosteroid therapy, including candidiasis, cataracts, glaucoma, osteoporosis, bone fractures, diabetes, and pneumonia. These ICS-related systemic side effects are of particular importance in elderly patients due to the presence of comorbidities. This is also true for inhaled bronchodilators, which can increase the risk of cardiovascular events in patients at high risk. Further, it is infrequent that the lack of asthma control is a direct consequence of a condition of drug-resistant disease. It is more likely that an uncontrolled condition is related to the underestimation of disease severity by physicians and patients with consequent prescription of insufficient medications and a low degree of treatment adherence. Inflammatory and structural changes of the peripheral airways are also for lack of control in mild stages of asthma.

Managing chronic obstructive respiratory diseases is to use inhalation therapy to access the target site while directing delivery of the aerosolized drug to the airways to treat inflammation and relieve the obstruction. The inhaled route of administration aims to exploit the topical effect directly near the deposition site, minimizing the risk of systemic exposure. Indeed, oral administration carries the risk of adverse events due to the high bioavailability of the drug. To improve the deposition in the periphery of the bronchial tree, the aerodynamic size of a particle should be first established and modulated by the type of device used, the aerosol formulation, and the patient's inhalation technique.

The targeted site is obtained by complying with three conditions: 1) the drug delivery system assures generating an aerosol cloud containing particles able to penetrate the respiratory tract; 2) the aerosol formulation enables the drug to deposit along the respiratory tract, and 3) the deposition of the drug should translate into functional and clinical benefits.

The drug contained in the pressurized MDI (pMDI) is usually formulated as a suspension or a solution, which may variably affect the delivery characteristics, such as particle size, plume velocity, and duration. Because of the fast-moving aerosol produced with the pMDI, the risk of depositing the drug in the pharynx is high, thus reducing the clinical efficacy. The need to be shaken before inhalation allows the uniform distribution of the drug's solid powder particles to favor the homogeneity of the drug concentration and result in dose consistency. Moreover, the suspension formulations may release large particles (due to agglomerating the micronized ones), of which only 10%-15% of the total dose achieves the conductive and lower peripheral airways.⁴² Because of the particle size, suspension formulations need a relatively larger orifice diameter to avoid its blocking by the emitted suspension, leading to higher velocity and lower duration of the aerosol plume.

In certain embodiments, DPI is generally favored in these patients over p/MDI with spacer or nebulizer if possible. DPIs formulations consist of micronized drug particles blended with larger carrier particles, typically a-lactose monohydrate, which enhance powder flowability, dispersion and reduces particle agglomeration. Replacing chlorofluorocarbon (CFC) propellants with hydrofluoroalkane (HFA) has allowed a shift from suspension formulations to solution formulations, where the drug is more uniformly distributed and the shaking of the inhaler is no longer necessary. Thus, the new HFA solution aerosols allow the particle size to be modified within the respirable range (extrafine particles or coarser ones). This property leads to deeper penetration of the medication into the lung. For example, the Modulite® technology (Chiesi Farmaceutici SpA, Parma, Italy) can produce a slow-moving cloud containing particles of the required particle size. Modulite achieves this goal primarily by the geometry of the actuator orifice, influencing the cloud formation since a smaller orifice produces more refined spray with slow-moving clouds over a much longer period. The Modulite technology also uses a nonvolatile component to provide the targeted particle size. Indeed, the particle size depends on the drug concentration in a droplet, and other nonvolatile components added. Each droplet will eventually dry to result in a particle, the size of which depends on the concentration of the drug in the solution. These characteristics confirm that the Modulite technology can deliver drug particles within the desired range of particle sizes, allowing uniform distribution along the bronchial tree to reach the peripheral airways.

In dry powder inhalers (DPIs), the drug is not driven by the fuel but delivered by inhalation effort. Conversely, in certain embodiments, actuating the device and the amount of drug reaching the lower airways depends on the patient's peak inspiratory flow. In the elderly population, the ability to generate sufficient inspiratory flow across a DPI is compromised, irrespective of an obstructive airway alteration.

In certain embodiments, the one or more corticosteroids is administered from one of three types of DPIs: single-dose (e.g., HandiHaler® [Boehringer Ingelheim, Ingelheim, Germany]; Ultibro® Breezhaler® [Novartis International AG, Basel, Switzerland]; and Foradil® Aerolizer® [Merck Millipore, Billerica, Mass., USA]); multiple-dose (e.g., Advair Diskus® [GlaxoSmithKline plc, London, UK]); and reservoir (e.g., Symbicort Turbuhaler® [AstraZeneca plc, London, UK]) inhalers. MDIs and DPIs require different techniques that imply specific training for each device: slow and deep inhalation for the MDIs; and inhalation that is quick, powerful, and as deep as possible for the DPIs. The Fostair® NEXThaler® (Chiesi Farmaceutici SpA), the Relvar® (GlaxoSmithKline plc), and the DuoResp Spiromax® (Teva Pharmaceutical Industries Ltd, Petah Tikva, Israel) devices are intuitively designed multidose DPIs, conceived for straightforward open-inhale-close operation that ensures ease of use. Direct delivery of the aerosolized drug in the lower airways is advocated to treat inflammation and relieve obstruction in small airway disease patients.

Asthma control can be obtained in most patients with inhaled steroids alone or with LABAs. ICSs effectively control airway inflammation, reduce symptoms, improve quality of life and lung function, decrease airway hyperresponsiveness, reduce the frequency and severity of exacerbations, and reduce mortality. Randomized clinical trials with the LABA with corticosteroids have demonstrated that adding LABA to ICSs is more beneficial for asthma control and pulmonary function than increasing the dose of ICSs alone.

Distributing the drug along the bronchial tree translates into higher efficacy of the inhaled therapy for functional and clinical benefits and reducing adverse event rates. Single-agent extrafine formulations are on the market (beclomethasone, ciclesonide, and formoterol [F]), and one combination product is available (beclomethasone/F).⁴⁰ Extrafine solution pMDIs can deliver compounds with a mass median aerodynamic diameter smaller than that delivered by other available devices, significantly increasing peripheral airway drug deposition of the delivered dose.^{58,59} ICS extrafine formulations alone were superior to nonextrafine formulations in modulating functional and inflammatory parameters, reflecting small airway abnormalities. A2.6-fold nonextrafine beclomethasone dipropionate (BDP) improved in forced expiratory volume in the first second (FEV₁) as extrafine HFA-BDP.

BDP extrafine 100 μg/actuation alone is clinically equivalent to BDP nonextrafine pMDI 250μ/actuation in patients with moderate asthma, demonstrating a 1:2.5 equivalence ratio between extrafine and nonextrafine BDP. An open-label, 12-month randomized controlled trial showed that in asthmatic patients previously treated with nonextrafine CFC-BDP and switched to equipotent doses of extrafine HFA-BDP (one-half of the dose of the nonextrafine CFC-BDP), significantly improved in asthma quality of life, as than those patients who continued to receive nonextrafine CFC-BDP.

PASC/long COVID is a small airway disease and, thus, more similar to asthma. Studies show asthma seems to respond better to extrafine, in certain embodiments, the disease is treated with a decreased dose/extrafine of either steroid alone, ICS+LABA, or ICS+LABA+LAMA. For treating PASC/long COVID, a 1:2.5 dose equivalence for BDP draws the dose down to around 1.5-2-fold (50-33% of standard dose) for extrafine as defined by MMAD/particle size. A dose adjustment may not be needed for Symbicort Turbhaler, because it delivers a MMAD of 2.2 which is close to the extrafine <2.1 μM cutoff.

Ciclesonide also showed functional changes, suggesting effective peripheral airway penetration. Ciclesonide administration improved lung function and reduced airway hyperresponsiveness. In Ciclesonide demonstrated anti-inflammatory effects in the peripheral airways, as determined by decreased alveolar exhaled nitric oxide and reduced air trapping following a methacholine challenge in mild-to-moderate asthmatics. To date, no extrafine fixed combination of ciclesonide with LABA is available in the market.

The BDP/F HFA pMDI combination is an extrafine formulation, in which the BDP dose is 2.5-fold lower than the conventional BDP CFC product (100 m of BDP per actuation instead of 250 m of nonextrafine BDP). Reducing the BDP dose lowers the amount of drug deposited in the upper airway and contributes to systemic exposure, therefore potentially improving the efficacy/safety ratio. The efficacy of BDP/F fixed combination was first evaluated in a 3-month trial conducted in patients with moderate asthma who were still symptomatic despite receiving low-dose ICSs (up to 500 m/day BDP or equivalent). BDP/F given at one inhalation twice daily proved to be more effective at improving lung function than a double equipotent dosage of BDP nonextrafine.

BDP/F given as two inhalations twice daily was as effective as nonextrafine BDP and F administered via separate inhalers, and was superior to nonextrafine BDP alone in improving lung function. BDP/F was significantly superior to separate components for asthma control. After 24 weeks of treatment, extrafine BDP/F delivered by an HFA pMDI (400/24 μg) was superior in improving asthma control to combining the same drugs formulated as larger nonextrafine agents at equipotent doses (1,000 μg BDP+24 μg F). Moreover, in two randomized clinical studies with a similar design, extrafine combination of BDP/F was comparable to the nonextrafine combinations of budesonide (BUD)/F (Turbuhaler BUD/F) and fluticasone propionate/salmeterol (pMDI FP/S) at equipotent doses for lung function improvement as measured by morning peak expiratory flow changes. When functional parameters more related to peripheral airway abnormalities were considered, 12 weeks of extrafine BDP/F combination treatment was significantly superior to an equipotent dose of the nonextrafine FP/S combination in improving air trapping, estimated by a reduction in forced vital capacity. These results provide direct evidence of the extrafine ICS/LABA combinations' superiority over the nonextrafine combinations in improving small airway function. Data from clinical trials did not document any increased risk of systemic effects with either a single inhaled ICS extrafine formulation or a combination of ICS/LABA extrafine therapy compared with nonextrafine therapy. The more rapid bronchodilation with the extrafine formulation than with the FP/S group leads to more immediate relief of symptoms and a potentially greater adherence rate in clinical practice. The superiority of extrafine combination treatment in improving the functional parameters of the peripheral airways, as than nonextrafine combinations, was demonstrated in a double-blind, randomized study performed in 30 asthmatics for 12 weeks. The extrafine BDP/F combination treatment (either extra-fine beclomethasone/formoterol (BDP/F) 400/24 μg daily or fluticasone propionate/salmeterol (FP/S) 500/100 μg daily tended to be significantly superior to equipotent doses of nonextrafine FP/S in improving closing capacity measured by the single-breath N2 washout test. Tt significantly decreased the degree of airway hyperresponsiveness, suggesting a homogeneous distribution of the drug throughout the bronchial tree.

The proportion of patients achieving asthma control was significantly higher in the BDP/F extrafine group than in the nonextrafine BUD/F or FP/S group. Furthermore, this was achieved with a significantly lower ICS mean daily dose. This observation was confirmed in a larger observational study (PRospectIve Study on asthMA control [PRISMA] study⁷⁷), showing that the extrafine BDP/F combination was more efficacious than the larger particle BUD/F and FP/S formulations in achieving asthma control and improving quality of life, respectively.

In a study on patients with severe COPD, subjects were randomized to receive extrafine BDP/F (200/12 mg pMDI), BUD/F (400/12 mg DPI), or F (12 mg DPI) twice daily for 48 weeks. It was shown that the use of BDP/F for 48 weeks improved FEV₁ to the same degree as BUD/F with a nominal dose of beclomethasone, which was twofold lower than the equipotent daily dose of BUD.⁸²

In the FORWARD study, the aim was to test the superiority of extrafine BDP/F 100/6 mg, two inhalations twice a day, over extrafine F alone in severe COPD patients with a history of exacerbations. As than F alone, extrafine BDP/F was shown to significantly reduce the exacerbation rate, prolong the time to first exacerbation, improve lung function assessed by predose morning FEV1, and improve the St. George's Respiratory Questionnaire total score.

NEXThaler is an extrafine powder, multidose, breath-actuated inhaler incorporating a full-dose feedback system providing accurate dose metering and consistent full-dose release, independent of the respiratory flow. Therefore, the extrafine BDP/F fixed combination represents the only extrafine combination in both the pMDI and DPI formulations developed thus far. A scintigraphic study confirmed high lung deposition and homogeneous distribution throughout the entire bronchial tree, both in healthy subjects and asthma and COPD patients.

A higher percentage of asthma patients could use the NEXThaler without errors when than Diskus and Turbuhaler. In the same study, NEXThaler was rated as the easiest to open, prepare and set a dose, tell how many doses were left, and the easiest instructions for use to understand.

Patients received either extrafine BDP/FF/G 100/6/10 μg via pressurized metered-dose inhaler or non-extrafine budesonide/formoterol (BUD/FF) 160/4.5 μg via dry-powder inhaler, both administered as two puffs twice-daily for 24 weeks. They found that BDP/FF/G was superior to BUD/FF for pre-dose and 2-h post-dose FEV1 at Week 24 [adjusted mean differences 62 (95% CI 38, 85) mL and 113 (87, 140) mL; both p<0.001]. The annualized moderate/severe exacerbation rate was 43% lower with BDP/FF/G [rate ratio 0.57 (95% CI 0.42, 0.77); p<0.001]. 61.1% and 67.0% of patients with BDP/FF/G and BUD/FF/FF reported adverse events. Results were similar in the China subgroup. They concluded that in patients with COPD, FEV₁<50% and an exacerbation history despite maintenance therapy, treatment with extrafine BDP/FF/G improved bronchodilation and was more effective at preventing moderate/severe COPD exacerbations than BUD/FF.

An electric nebulizer was more efficacious for budesonide administration than dry powder delivery. The NE-C28 treatment deposited 2.36× more budesonide in the airway obstruction region than dry powder delivery systems. HFA+alcohol-pMDI is a super-small aerosol that can effectively reach and treat small airway obstructions. The MMADs of beclomethasone pMDI (Qvar, Sumitomo Dainippon Pharma Co., Ltd., Tokyo, Japan) and ciclesonide pMDI (Alvesco, Teijin Pharma, Tokyo, Japan) are 1.1 μm and 0.9 μm according to the manufacture's statement, respectively. Pulmonologists recommend that super-small steroid particles are efficacious in managing small airway (bronchial diameter <2 mm) obstruction. Accordingly, the use small aerosol released pMDI may be useful to small airway obstruction. The percentage of particles <2 μm in diameter deposited in the alveoli was 46.0% for the Turbuhaler, 42.0% for the Symbicort, 11.0% for the mesh-type NE-U22 suspension and 17% for the jet-type NE-C28 suspension. The percentages of particles <2 μm in diameter generated by the DPI (Pulmicort and Symbicort) were higher than those generated by the mesh- and jet-type electric nebulizers.

Thus, it is clear that these therapies (drug formulation and device) do not effectively reach the smaller airways in sufficient proportion to the total amount of drug delivered to effect the smaller peripheral airways that predominantly effect PASC/long COVID pulmonary patients as described.

### Example 12

Patients were initiated on standard pulmonary rehabilitation and prescribed ICS (inhaled corticosteroids) alone or in combination with B2 agonists (B2), including long-acting B2 agonists (LABA), including but not limited to, various formulations of budesonide, ciclesonide, mometasone, beclomethasone, or fluticasone, and/or a beta2 agonist chosen from formoterol, albuterol, levalbuterol, and salmeterol. Examples of prescribed ICS therapies to patients included, but were not limited to Advair Diskus at μg standard doses of 100 μg/50 μg, 250/50 or 500/50 one puff/actuation twice daily BID, or Seratide Diskus or Evohaler 50/25, 125/25, 250/25 with lactuation (BID), or Symbicort Turbohaler or Rapihaler/Vannair Inhaler or DuoResp Spiromax at standard doses of 80 μg/4.5 μg, 160/4.5, or 320/9-1-2 puffs/BID, as well as standard daily doses of other ICS such as Pulmocort, Flovent, QVAR, Alvesco, Arnuity, Asmanex, etc.). Patients who could not generate sufficient inspiratory flow to tolerate or effectively use a Dry Powder Inhaler (DPI) were then given similar medications and equivalent dosing via pressurized metered-dose inhaler (MDI) with a spacer or via nebulizer. For example, some patients were typically started on standard dosing with Symbicort, at standard dosing of 2 puffs/actuations BID. If the disease or symptoms were not adequately controlled, they were stepped up to 3 puffs BID and up to a maximum of 4 puffs BID. Other examples of dosing included modulating dosing using these standard dosing formulations as previously mentioned, achieving BID dosing and disease control while limiting steroid dose to the equivalent maximum of 160 μg×8 total received daily in a BID scheduling, and/or a formoterol maximum tolerated no greater than 4.5 ug×8 total received daily over BID schedule. These were typically adjusted and titrated over the initial 1-2 weeks and stepped up over two-week increments if stepping up was required, followed by a similar step-down strategy.

Treatments were largely dose-dependent. Those could be modulated accordingly and were strongly correlated with response. A similar strategy was applied to other ICS/B2 combination therapies, such as Seratide and Advair, where patients were typically started at standard dosing of Seretide Evohaler 25 microgram salmeterol/50 microgram of fluticasone dose inhalation powder, 25/125 or 25.250, pre-dispensed at 1-2 puffs/actuations BID or Seretide Diskus Accuhaler 50 microgram salmeterol/100 microgram of fluticasone dose inhalation powder, 50 microgram/250 microgram/dose inhalation powder, or 50 microgram/500 microgram/dose inhalation powder, pre-dispensed at 1 puff/actuation BID. If the disease or symptoms were not adequately controlled, they were stepped up to 2 puffs BID. This strategy broadly applied to all medications in this class listed.

If disease/symptoms were still not adequately controlled on these medications, then patients were switched to oral steroids, including but not limited to prednisolone, betamethasone, dexamethasone, methylprednisolone, and hydrocortisone, and followed up a similar step to achieve disease control while mitigating for drug-related adverse events, and then eventually stepped down/weaned and then switched to inhaled steroid therapy as described above. Upon follow up, which ranged from 1 week to multiple months post-therapy initiation for post-acute covid pulmonary sequelae, the vast majority of patients reported significant improvement both subjectively and objectively in their symptoms and disease controlled as assessed by standard measures such as mMRC, SGRQ, as well as in other measures such as 6 min walk tests, subjective assessments of daily walking distance, number of flights of stairs before experience dyspnea, ability to carry out daily activities, ability to walk versus drive, and related measures. These and objective measures guided treatment selection, dose, and dosing schedule. Patients typically were on therapy for 3-6 months on average before stepping down/weaning off. Some patients were on therapy at three months, and others extended to one year or maintained therapy beyond one year. The patient generally either was able to wean ICS-based therapy, with the remaining preferring to stay on therapy or have the therapy available PRN. Overall, the treatment followed a general strategy of initiation of therapy, followed by titration of therapeutic benefit to the adverse event while minimizing adverse events followed by maintenance at this dose until the therapeutic objective was achieved, followed by a step-down.

Objective data including but not limited to peak flow assessment, spirometry, inspirometry, and imaging such as chest x-ray, CT imaging, MRI data on these COVID patients at presentation showed FVC deficits from pre-infection baseline in the range of up 30-50% for most post-acute COVID symptomatic patients (severe patients tended to have a higher loss, while not severe tended to have less loss relative to severe) with FEV1 ranges in the same loss territory; FEF 25, 50 and 75 showed small airways involvement and some gas trapping was generally observed, as well as imaging findings consistent as seen and previously described by Cho et al. above, that together, was most consistent with small airways disease as a prominent feature, with components of air trapping, obstruction, and inflammation. Up similar deficits in DLco/Kco, when corrected for volume, were also observed. Findings on imaging were also consistent as described herein with COVID, including a range consisting of peripheral ground-glass opacities, areas of consolidation, fibrosis, bronchiectasis, inflammation, interstitial lung changes, and air trapping.

While there was a general correlation between the severity of pulmonary deficit during acute infection and deficit and pulmonary function, this was not absolute. There were cases where patients with mild or moderate disease had more profound deficits, and vice versa for patients with severe acute disease, similar to Cho at Example 10. Overall, while on their ICS/B2 therapy treatment course, these objective and subjective values generally significantly improved, with subjective measures significantly improved per patient report (a significant improvement on SGRQ or mMRC, in performance of daily activities, etc.). After therapy, when patients have completely weaned off these medications or using them infrequently or PRN, the significant majority of patients objective pulmonary function across the measures showing deficit had either stabilized (particularly in the most severe patients) and/or significantly improved, with absolute margins of improvement up to or exceeding 15-20% on average (relative improvements on average of 30%), and with some patients having more profound recovery with complete reversion to baseline/normal levels, in these objective pulmonary function measures. Parallel concordant findings were also often noted on imaging as well.

A subset of patients continued to subjectively and objectively progress in symptoms worsening and functional loss in the subsequent 1-3 months post-discharge/post-acute COVID phase. Further, intervention as described with these treatments in those with either evidence of post-acute COVID pulmonary sequelae or who were at high risk of developing post-acute COVID pulmonary symptoms—such treatments benefited these patients allowing for both a faster and more robust recovery than those that did not receive such treatment. Indeed, these patients continued to lose an additional 10-15% of absolute lung function (beyond their initial loss experienced during the acute infection phase) over the initial 1-3 months post-acute infection time. Conversely, those who did not receive these treatments typically did not recover the initial acute phase lung function loss or this other post-acute phase lung function loss. Conversely, those that were treated showed either a stabilization without experiencing the further post-acute covid lung function loss or even achieved significant improvement or recovery of lung function (i.e., they did not have the additional 10-15% loss in their intervening 1-3 months post-acute phase or were able to recover from that 10-15% post-acute phase lung loss, or were able to achieve lung function recovery beyond their initial post-acute functional lung loss, and/or were able to functionally, subjectively, objectively recover completely). Thus, early treatment of patients with either patient that had post-acute pulmonary sequelae from COVID or with high likelihood or risk for development benefitted from the aforementioned treatment regimens and strategies.

However, such standard treatments as described of these post COVID patients could also lead to increased risk of infection due to high steroid dose (oral infections including thrush, and pneumonia, high glucose and hypothalamic-pituitary-adrenal related effects, cataracts), B2 related exacerbations related to high B2 dosing and/or LABAs (tremors, headaches, palpitations, tachycardia, dizziness, chest pain, glaucoma, hypokalemia, dry throat and throat irritation, hoarseness, cough, angioedema, etc.) or poor control of their pulmonary symptoms/disease—even at high doses. In general, rate-limiting effects were more often titrated by controlling for excessive steroid dose those selected for the best steroid and dose with likely lowest complication profile, unless the patient was suffering from or at risk for B2 related side effects. Post-acute COVID patients, in general, reported significant immediate relief and overall benefit with little side effects from ICS+LABA based treatment as described. As a result, therapies had to be titrated down or switched to other therapies either within the same class or, in some cases, switched to different classes of therapies.

The biological, physiological, and immunological profiles of these post-COVID pulmonary patients based on these data suggested some features for asthma and hyperreactive airways disease, and COPD, yet also facets that appear nonoverlapping as well, with features for a more pronounced immune/autoimmune and hyperreactivity like component most notably focused in the peripheral/small airways all which prompted and guided these treatment strategies disclosed. Further, the cough component also is more pronounced and appears to play a significant role in post-COVID morbidity in these patients than was observed in typical asthma and COPD, which could often exacerbate, initiate, or worsen the symptoms of dyspnea, shortness of breath, cough, and chest pain. Indeed, the ability of a DPI to improve post-COVID cough and airways bronchial hyper-reactivity (BHR) or cough-variant asthma/airways (CVA) like features is an important component of the treatment strategy benefit patients

Thus, there is a significant degree of variability and inflammation-driven airways hypersensitivity, more dominant in the peripheral lung distributions and cough, in post-COVID patients that makes many elements of these post-acute sequelae reversible in the early months post-discharge as shown, by managing their treatment of both these overlapping and unique pathophysiologic features in post-COVID patients as described herein.

Other critical factors include, but are not limited to, median mass aerodynamic diameter (MMAD), particle size, geometric standard deviation (GSD), and simple to administer uniform drug cloud dispersion to effectively deliver these drug particles beyond the oropharynx and large airways, to the small airways are also factors in the mechanics of drug-delivery and efficiency of function. Some of this might be abrogated by higher dosing with nonextrafine (MMAD >2.1 μM) drug compositions alone or with more effective inhaler devices as described herein, but as shown, extrafine compositions (MMAD <2.1 μM or GSD <1.2) alone or with these newer inhaler devices (Fostair Nexhaler at 100/6 ug or 200/6 ug BID but stepping up or down as previously described), provide more uniform, homogenous, targeted distribution to the small airways and at lower doses with less adverse effects. Indeed, selecting the drugs (e.g., inhaled steroid+/−B2 agonist or LABA+/−antimuscarinic or LAMA)+dosage+drug particle size (MMAD, GSD)+solvent (HFA)+drug delivery device (pMDI, DPI, nebulizer, other) must target the small airways and is of particular relevance to the treatment of post-acute pulmonary sequelae of COVID. Particularly to deliver the minimum effective dose to the targeted regions to effect abrogation or mitigation of these disease processes as disclosed herein, including, but not limited to, selecting an ICS+/−LABA+/−LAMA, based on patient needs. For example, the MMAD and/or GSD are extrafine (<2.1 μM)/<1.2 respectively, and thus doses can be reduced potentially up to 50%, with an easy to use device that can distribute and disperse the drug and solvent uniformly to the peripheral lungs with lower plume velocities and over a significant time duration.

The key issues otherwise noted are peak-inspiratory airflow velocity and residual lung volumes. For DPI's this is a critical marker of whether a patient could inspire deeply and quickly enough to “pick up” the necessary, correct drug dosing. In certain embodiments, when the patient has decreased peak/inspiratory flows objectively or subjectively, the drug is delivered with an MDI with a spacer or pMDI. In those instances where these factors were rate-limiting (seen more frequently in the elderly, patients with more severe acute COVID, underlying comorbidities, etc.), DPIs were substituted for MDIs with spacers, nebulizers, and those next-generation inhalers (e.g., NeXT haler), as well as extrafine formulations (Fostair), are of benefit.

### Example 13

Some post-COVID patients, as previously described, were also treated with inhaled triple therapies (inhaled steroid, B2 agonist or long-acting B2 agonist (LABA), anti-muscarinic or long-acting anti-muscarinic agents (LAMA)) or LABA/LAMA or LABA only therapies with similar beneficial effect as described in the previous examples. These included but were not limited to patients with underlying COPD or emphysema or that had other features that suggested these disease-like features, smokers/past-smokers, pronounced shortness of breath or cough, productive coughs, or wheezing, or that were resistant to the dual ICS/B2 therapies. These included but were not limited to formulations including fluticasone, vilanterol umeclidinium at doses of 100/25/62.5 ug respectively, or 200/25/62.5 and Trelegy (Trelegy Ellipta) and its variants dosed one puff/actuation daily for MDIs or DPIs. Other triple therapies include, but are not limited to, other established ICS/LABA (long-acting B2 agonist agents)/LAMA combinations, which were used to similar effect. Wheezing and shortness of breath typically resolved, as did other features and subjective and objective features as previously described within the similar treatment and dosing/tapering schedules and time frames. However, similarly, there is an even higher risk of infection (e.g., thrush, pneumonia, etc.) in addition to the B2 agonism effects as previously described, as well as anti-muscarinic effects (dry mouth; constipation; blurred vision; drowsiness; nausea; vomiting; abdominal discomfort; difficulty micturating; palpitations; skin reactions). Thus, in some instances, patients benefit from less fluticasone dose to mitigate increased infection risks, or a lower dose and more localized delivery as afforded by extrafine drug particles, and or other steroids (e.g., beclomethasone), or LAMAs (tiotropium bromide 18 μg, and/or next-generation delivery devices or devices or formulations that allow more uniform drug delivery to the small airways, or both (e.g., Fostair pressurized MDI or NeXT haler (100/6 or 200/6 mg beclomethasone/formoterol, or Spiriva Respimat or Handihaler, Bevespri Aerosphere glycopyrrolate/formoterol 9/4.8 ug BID), Spiolto Respimat, Spriva or Berodual, or Enerzair Breezehaler).

### Example 14

Some patients with severe post-acute COVID had severe sequelae with pulmonary function and/or medical imaging studies suggesting a severe inflammatory, fibrotic, or restrictive lung disease component. Aspects shared some features with interstitial lung disease, some restrictive pneumoconioses, changes in the lung seen with rheumatoid arthritis, lupus, systemic sclerosis, ankylosing spondylitis, and similar related autoimmune conditions related lung diseases. Imaging showed in some radiological changes of interstitial thickening, honeycombing, bronchiectasis, ground-glass opacities, reticulations, peribronchial thickening, and features seen in interstitial pulmonary fibrosis, usual, nonspecific, desquamative, respiratory bronchiolitis, acute, or cryptogenic interstitial pneumonia changes. While the primary treatment goal was symptom/disease control, a primary concern is limiting progressive fibrosis radiologically (and pathologically and functionally/symptomatically) as a significant fraction of patients with a severe loss in pulmonary function and evidence of fibrosis continued to progress even after their acute infection/stabilization phase over the next 1-3 months. As such, patients benefited from anti-fibrotic agents (e.g., standard dosing of pirfenidone (267 mg tablets, 1 pill TID, stepped up after week one to 2 pills TID, then 3 pills TID, or nintedanib 100 or 150 mg BID). The patient was also frequently concomitantly treated with ICS-based therapies as previously described (e.g., ICS/LABA or ICS/LABA/LAMA, etc.).

To limit side effects, doses were of their anti-fibrotic medication were often significantly reduced, particularly in Asian patients, where doses could be reduced by as much as up to ⅓ of standard dosing (e.g., pirfenidone maximum dose one pill TID or (3 pills total daily), nintedanib 100 mg BID). Treating these patients in general and those requiring dose adjustment were well tolerated without major or significant adverse events. In general, patients that did not receive any treatment ICS-based therapy+/−anti-fibrotic agent tended to hit and stabilize at a nadir, while those treated with these therapies tended to see a 10-15% absolute improvement (or 30% relative improvement) on the whole. Further, CT imaging on follow-up generally revealed stabilization of disease or even mild improvement could be observed as early as 2-3 months post-therapy initiation, as well as a symptom/functional recovery or stabilization. Some patients continued maintenance therapy on these medications, while others could be weaned off. Patients were then either maintained on their ICS-based therapies according to similar schedules and requirements as described in the previous examples enclosed herein.

### Example 15.—Treating PASC Pulmonary Patients with Existing Treatments Using Standard Dosing and Delivery Devices

Following the methods disclosed herein, patients can be treated for PASC pulmonary sequalae using existing therapies of inhaled corticosteroids (Table 2), inhaled coformulations of corticosteroids and LABAs (Table 3), and inhaled coformulations of corticosteroids, LABAs, and an antimuscarinic agent (Table 4).

TABLE 2
Dosing instructions for treating PASC pulmonary patients with
standard dosing and delivery devices of inhaled corticosteroids

| Name and | Drug | Method of | | Delivered | Dosing with | Steroid Max |
|---|---|---|---|---|---|---|
| Manufacturer | component | delivery | Diluent | dosing | COPD | daily dose |
| Qvar RediHaler | beclomethasone | pressurized, | HFA- | adults: (1 or 2 puffs) | | 320 μg BID |
| (Teva) | dipropionate | metered-dose | 134a, Ethanol, | 40 μg to 80 μg BID | | |
| | | aerosol | Dehydrated | | | |
| Qvar Autohaler | beclomethasone | pressurized, | HFA- | adults: (1 or 2 puffs) | | 320 μg BID |
| (Teva) | dipropionate | metered-dose | 134a, Ethanol | 40 μg to 80 μg BID | | |
| | | aerosol | | | | |
| Flovent HFA | fluticasone | pressurized, | HFA-134a | adults: 2 puffs 88 μg | | 1320 μg BID |
| (GSK) | propionate | metered-dose | | BID | | |
| | | aerosol | | | | |
| Flixotide | fluticasone | pressurized, | HFA-134a ( | adults: 2 puffs 100 | adults: 2 puffs | 1320 μg BID |
| Evohaler (GSK) | propionate | metered-dose | | μg BID | 250 μg BID | |
| | | aerosol | | | | |
| Flixotide Diskus | fluticasone | dry powder | lactose | adults: 2 puffs (100 | adults: 2 puffs | 1320 μg BID |
| (GSK) | propionate | inhaler | monohydrate | μg to 250 mg) BID | of 250 mg | |
| | | | | | BID | |
| Flovent Rotadisc | fluticasone | dry powder | lactose | adults: 1 puff 100 | | 1320 μg BID |
| (GSK) | propionate | inhaler | monohydrate | μg BID | | |
| Arnuity Ellipta | fluticasone | dry powder | lactose | adults: 1 puff 100 | adults: 1 puff | 400 μg once |
| (GSK) | furoate | inhaler | monohydrate | μg once daily | 100 μg once | daily |
| | | | | | daily | |
| ArmonAir | fluticasone | dry powder | lactose | adults: 2 puffs 55 μg | | 1320 μg BID |
| Digihaler (Teva) | propionate | inhaler | monohydrate | BID | | |
| Pulmicort | budesonide | dry powder | lactose | adults: 2 puffs 180 | | 800 μg BID |
| Flexhaler | | inhaler | monohydrate | μg BID | | |
| (AstraZenaca) | | | | | | |
| Pulmicort | budesonide | dry powder | / | adults: 1 puff (200 | | 800 μg BID |
| Turbohaler | | inhaler | | μg) BID | | |
| (AstraZeneca) | | | | | | |
| Alvesco (Covis) | ciclesonide | pressurized, | HFA- | adults: 1 or 2 puffs | | 320 μg BID |
| | | metered-dose | 134a, Ethanol | (80 μg to 160 μg) | | |
| | | aerosol | | BID | | |
| Asmanex | mometasone | dry powder | lactose | adults: 2 puffs (100 | adults: 2 puffs | 220 μg BID |
| Twisthaler | | inhaler | monohydrate | μg to 200 μg) BID | (100 μg) BID | |
| (Organon Global) | | | | | | |
| Asmanex HFA | mometasone | pressurized, | HFA-227 | adults: 2 puffs (100 | adults: 2 puffs | 220 μg BID |
| (Organon Global) | | metered-dose | | μg to 200 μg) BID | (100 μg) BID | |
| | | aerosol | | | | |

TABLE 3
Dosing instructions for treating PASC pulmonary patients with standard
dosing and delivery devices of inhaled corticosteroids with LABA

| Name and | Drug | Method of | | Delivered | Dosing with | Steroid Max |
|---|---|---|---|---|---|---|
| Manufacturer | component | delivery | Diluent | dosing | COPD | daily dose |
| Symbicort | budesonide & | DPI (dry powder | lactose | adults: 1-2 puffs | adults: 2 puffs | 800 μg BID |
| (AstraZeneca) | formoterol | inhaled), MDI with | monohydrate | (160/4.5 μg) | (160/4.5 μg) BID | |
| | fumarate dihydrate | spacer, nebulizer | | BID | | |
| Duoresp Spiromax | budesonide & | dry powder inhaler | lactose | adults: 1-2 puffs | 1 puff (160/4.5 | 800 μg BID |
| (Teva) | formoterol | | monohydrate | (160/4.5 μg) | μg)BID | |
| | fumarate dihydrate | | | BID | | |
| Atectura | mometasone & | Inhalation powder, | lactose | adults: 1 puff | — | 220 μg BID |
| Breezhaler | indacaterol | hard capsule | monohydrate | (125 μg | | |
| (Novartis) | | (inhalation | | mometasone | | |
| | | powder) | | with 62.5, 127.5, | | |
| | | | | or 260 μg | | |
| | | | | indacaterol) | | |
| | | | | once daily | | |
| FOSTAIR | beclomethasone | inhalation powder | Norflurane (HFA- | adults: 1-2 puffs | 2 puffs (84.6/5 | 169.2 μg BID |
| NEXThaler | dipropionate & | | 134a) | (84.6/5 μg) BID | μg) BID | |
| (Trinity Chiesi) | formoterol | | | | | |
| | fumarate dihydrate | | | | | |
| Advair Diskus | fluticasone | Inhalation powder | lactose | — | adults: 1 puff (92, | 1320 μg BID |
| (GSK) | propionate & | | monohydrate | | 233 or 465 μg | |
| | salmeterol | | | | with 45 μg) BID | |
| | xinafoate | | | | | |
| Advair HFA | fluticasone & | pressurized | 1,1,1,2- | adults: 2 puffs | Same as non- | 230 μg BID |
| (Teva) | salmeterol | inhalation, | tetrafluoroethane | (45, 115, or 230 | COPD | |
| | | suspension | (HFA-134a) | fluticasone with | | |
| | | | | 21 μg | | |
| | | | | salmeterol) BID | | |
| Airduo Respiclick | fluticasone & | Inhalation powder | lactose | adults: 1 puff | Same as non- | 1320 μg BID |
| (Teva) | salmeterol | | monohydrate | (49, 100, or 202 | COPD | |
| | | | | μg fluticasone | | |
| | | | | with 12.75 μg | | |
| | | | | salmeterol) BID | | |
| Airduo Digihaler | fluticasone & | Inhalation powder | lactose | adults: 1 puff | Same as non- | 1320 μg BID |
| (Teva) | salmeterol | | monohydrate | (49, 100, or 202 | COPD | |
| | | | | μg fluticasone | | |
| | | | | with 12.75 μg | | |
| | | | | salmeterol) BID | | |
| Wixela Inhub | fluticasone & | Inhalation powder | lactose | — | adults: 1 puff (93, | 1320 μg BID |
| (Mylan) | salmeterol | | monohydrate | | 233, or 465 μg | |
| | | | | | fluticasone with | |
| | | | | | 45 μg salmeterol) | |
| | | | | | BID | |
| Seretide diskus | fluticasone & | Inhalation powder | lactose | adults: 1 puff | Same as non- | 1320 μg BID |
| (GSK) | salmeterol | | monohydrate | (92, 231, or 460 | COPD | |
| | | | | μg fluticasone | | |
| | | | | with 47 μg | | |
| | | | | salmeterol) BID | | |
| Aerivio ® | fluticasone & | Inhalation powder | lactose | adults: 1 puff | Same as non- | 1320 μg BID |
| Spiromax (Teva) | salmeterol | | monohydrate | (465/45) BID | COPD | |
| Sirdupla (Mylan) | fluticasone & | pressurized | HFA 134a | adults: 2 puffs | — | 1320 μg BID |
| | salmeterol | inhalation, | | (220/21) BID | | |
| | | suspension. | | | | |
| Airflusal Forspiro | fluticasone & | Inhalation powder | lactose | adults: 1 puff | Same as non- | 1320 μg BID |
| (Aeropharm) | salmeterol | | monohydrate | (465/45 μg) BID | COPD | |
| Breo Ellipta | fluticasone & | Inhalation powder | lactose | adults: 1 puff | Same as non- | 1320 μg BID |
| (GSK) | vilanterol | | monohydrate | (92 or 184 μg | COPD | |
| | | | | fluticasone with | | |
| | | | | 22 μg vilanterol) | | |
| | | | | once daily | | |
| Dulera (Merck) | mometasone & | pressurized, | anhydrous | adults: 2 puffs | Same as non- | 220 μg BID |
| | formoterol | metered-dose | alcohol | (100 or 200 μg | COPD | |
| | | aerosol | | mometasone | | |
| | | | | with 5 μg | | |
| | | | | formoterol) BID | | |

TABLE 4
Dosing instructions for treating PASC pulmonary patients with standard dosing and
delivery devices of inhaled corticosteroids with LABA and an antimuscarinic agent

| Name and | Drug | Method of | | Delivered | Dosing with | Steroid Max |
|---|---|---|---|---|---|---|
| Manufacturer | component | delivery | Diluent | dosing | COPD | daily dose |
| Trelegy Ellipta | fluticasone & | Inhalation powder | lactose | — | adults: 1 puff | 1320 μg BID |
| (GSK) | vilanterol | | monohydrate | | (92/55/22 μg) | |
| | trifenatate & | | | | once daily | |
| | furoateumeclidinium | | | | | |
| | bromide | | | | | |
| Breztri aerosphere | budesonide & | pressurized, | HFA 134a | — | adults: 2 puffs | 800 μg BID |
| (AstraZeneca) | formoterol & | metered-dose | | | (160/9/4.8 μg) | |
| | glycopyrrolate | aerosol | | | BID | |
| Trixeo Aerosphere | budesonide & | pressurized, | Norflurane 1,2- | — | adults: 2 puffs | 800 μg BID |
| (AstraZeneca) | formoterol & | metered-dose | distearoyl-sn- | | (160/7.2/5 μg) | |
| | glycopyrrolate | aerosol | glycero-3- | | BID | |
| | | | phosphocholine | | | |
| Trimbow (Trinity | beclomethasone & | pressurized | Ethanol | adults: 2 puffs | Same as non- | 320 μg BID |
| Chiesi) | formoterol fumarate | inhalation, solution | anhydrous | (87/5/9 μg) BID | COPD | |
| | dihydrate & | | | | | |
| | glycopyrronium | | | | | |
| | bromide | | | | | |
| Enerzair | mometasone & | inhalation powder, | lactose | adults: 1 puff | — | 220 μg BID |
| Breezhaler | indacaterol & | hard capsules | monohydrate | (136/114/58 μg) | | |
| (Novartis) | glycopyrronium | | | once daily | | |
| FF/UMEC/VI | fluticasone furoate & | pressurized | — | — | adults: 1 puff | 400 μg |
| (GSK) | vilanterol & | inhalation, solution | | | (100/25/62.5 | once daily |
| | umeclidinium | | | | μg) once daily | |
| | bromide | | | | | |
| B/F/G (Trinity | beclomethasone & | pressurized | — | adults: 1 puff | — | 320 μg BID |
| Chiesi) | formoterol & | inhalation, solution | | (200/6//12.5 μg) | | |
| | glycopyrronium | | | once daily | | |

### Example 16.—Treating PASC Pulmonary Patients with Modifying Doses and Devices of Existing Therapies

Following the methods disclosed herein, patients can be treated for PASC pulmonary sequalae using modified doses and devices existing therapies of inhaled corticosteroids, inhaled coformulations of corticosteroids and LABAs, and inhaled coformulations of corticosteroids, LABAs, and an antimuscarinic agent (Table 5).

TABLE 5
Dosing instructions for treating PASC pulmonary patients with
modified dosing and delivery devices of inhaled corticosteroids

| Name and | Steroid | | | | Steroid Max | |
|---|---|---|---|---|---|---|
| Manufacturer | component | Diluent | Method of delivery | Delivered dosing | Daily | |
| Qvar RediHaler | beclomethasone | HFA- | pressurized, metered- | adults: 1 or 2 puffs | 320 | μg BID |
| (Teva) | dipropionate | 134a, Ethanol, | dose aerosol | (30-60 μg) BID | | |
| | | Dehydrated | | | | |
| Qvar Autohaler | beclomethasone | HFA- | pressurized, metered- | adults: 1 or 2 puffs | 320 | μg BID |
| (Teva) | dipropionate | 134a, Ethanol | dose aerosol | (30-60 μg) BID | | |
| Flovent HFA | fluticasone | HFA-134a | pressurized, metered- | adults: 2 puffs (40- | 1320 | μg BID |
| (GSK) | propionate | | dose aerosol | 300 μg) BID | | |
| Flixotide | fluticasone | HFA-134a | pressurized, metered- | adults: 2 puffs (40- | 1320 | μg BID |
| Evohaler (GSK) | propionate | | dose aerosol | 300 μg) BID | | |
| Flovent Diskus | fluticasone | lactose | dry powder inhaler | adults: 2 puffs (40- | 1320 | μg BID |
| (GSK) | propionate | monohydrate | | 300 μg) BID | | |
| Flixotide Diskus | fluticasone | lactose | dry powder inhaler | adults: 2 puffs (40- | 1320 | μg BID |
| (GSK) | propionate | monohydrate | | 300 μg) BID | | |
| Flovent Rotadisc | fluticasone | lactose | dry powder inhaler | adults: 1 puffs (40- | 1320 | μg BID |
| (GSK) | propionate | monohydrate | | 300 μg) BID | | |
| Arnuity Ellipta | fluticasone | lactose | dry powder inhaler | adults: 1 puffs (40- | 400 μg once | |
| (GSK) | furoate | monohydrate | | 300 μg) BID | daily | |
| ArmonAir | fluticasone | lactose | dry powder inhaler | adults: 2 puffs (40- | 1320 | μg BID |
| Digihaler (Teva) | propionate | monohydrate | | 300 μg) BID | | |
| Pulmicort | budesonide | lactose | dry powder inhaler | adults: 2 puffs (30- | 800 | μg BID |
| Flexhaler | | monohydrate | | 450 μg) BID | | |
| (AstraZeneca) | | | | | | |
| Pulmicort | budesonide | / | dry powder inhaler | adults: 1 (70-250 | 800 | μg BID |
| Turbohaler | | | | μg) puff BID | | |
| (AstraZeneca) | | | | | | |
| Alvesco (Covis) | ciclesonide | HFA- | pressurized, metered- | adults: 1 or 2 puffs | 320 | μg BID |
| | | 134a, Ethanol | dose aerosol | (70-250 μg) BID | | |
| Asmanex | mometasone | lactose | dry powder inhaler | adults: 2 puffs (70- | 220 | μg BID |
| Twisthaler | | monohydrate | | 360 μg) BID | | |
| (Organon Global) | | | | | | |
| Asmanex HFA | mometasone | HFA-227 | pressurized, metered- | adults: 2 puffs (70- | 220 | μg BID |
| (Organon Global) | | | dose aerosol | 360 μg) BID | | |
| Symbicort | budesonide | lactose | DPI (dry powder | adults: 1-2 puffs | 800 | μg BID |
| (AstraZeneca) | | monohydrate | inhaled), MDI with | (30-350 μg) BID | | |
| | | | spacer, nebulizer | | | |
| Duoresp | budesonide | lactose | dry powder inhaler | adults: 1-2 puffs | 800 | μg BID |
| Spiromax (Teva) | | monohydrate | | (70-360 μg) BID | | |
| Atectura | mometasone | lactose | Inhalation powder, hard | adults: 1 puff (60- | 220 | μg BID |
| Breezhaler | | monohydrate | capsule (inhalation | 360 μg) once daily | | |
| (Novartis) | | | powder). | | | |
| FOSTAIR | beclomethasone | Norflurane (HFA- | inhalation powder | adults: 1-2 puffs | 169.2 | μg BID |
| NEXThaler | dipropionate | 134a) | | (30-160 μg) BID | | |
| (Trinity Chiesi) | | | | | | |
| Advair Diskus | fluticasone | lactose | Inhalation powder | adults: 1-2 puffs | 1320 | μg BID |
| (GSK) | propionate | monohydrate | | (40-300 μg) BID | | |
| Advair HFA | fluticasone | 1,1,1,2- | pressurized inhalation, | adults: 2 puffs (40- | 230 | μg BID |
| (Teva) | | tetrafluoroethane | suspension | 300 μg) BID | | |
| | | (HFA-134a) | | | | |
| Airduo Respiclick | fluticasone | lactose | Inhalation powder | adults: 1 puff (40- | 1320 | μg BID |
| (Teva) | | monohydrate | | 300 μg) BID | | |
| Airduo Digihaler | fluticasone | lactose | Inhalation powder | adults: 1 puff (40- | 1320 | μg BID |
| (Teva) | | monohydrate | | 300 μg) BID | | |
| Wixela Inhub | fluticasone | lactose | Inhalation powder | adults: 1 puff (40- | 1320 | μg BID |
| (Mylan) | | monohydrate | | 300 μg) BID | | |
| Seretide diskus | fluticasone | lactose | Inhalation powder | adults: 1 puff (40- | 1320 | μg BID |
| (GSK) | | monohydrate | | 300 μg) BID | | |
| Aerivio ® | fluticasone | lactose | Inhalation powder | adults: 1 puff (40- | 1320 | μg BID |
| Spiromax (Teva) | | monohydrate | | 300 μg) BID | | |
| Sirdupla (Mylan) | fluticasone | HFA 134a | pressurized inhalation, | adults: 1 puff (40- | 1320 | μg BID |
| | | | suspension. | 300 μg) BID | | |
| Airflusal Forspiro | fluticasone | lactose | Inhalation powder | adults: 1 puff (40- | 1320 | μg BID |
| (Aeropharm) | | monohydrate | | 300 μg) BID | | |
| Breo Ellipta | fluticasone | lactose | Inhalation powder | adults: 1 puff (40- | 1320 | μg BID |
| (GSK) | | monohydrate | | 300 μg) BID | | |
| Relvar Ellipta | fluticasone | lactose | Inhalation powder | adults: 1 puff (40- | 1320 | μg BID |
| (GSK) | | monohydrate | | 360 μg) BID | | |
| Dulera (Merck) | mometasone | anhydrous | pressurized, metered- | adults: 2 puffs (70- | 220 | μg BID |
| | | alcohol | dose aerosol | 360 μg) BID | | |
| Trelegy Ellipta | fluticasone | lactose | Inhalation powder | adults: 1 or 2 puffs | 1320 | μg BID |
| (GSK) | | monohydrate | | (40-300 μg) BID | | |
| Breztri aerosphere | budesonide | HFA 134a | pressurized, metered- | adults: 1 or 2 puffs | 800 | μg BID |
| (AstraZeneca) | | | dose aerosol | (30-350 μg) BID | | |
| Trixeo | budesonide | Norflurane 1,2- | pressurized, metered- | adults: 1 or 2 puffs | 800 | μg BID |
| Aerosphere | | distearoyl-sn- | dose aerosol | (30-350 μg) BID | | |
| (AstraZeneca) | | glycero-3- | | | | |
| | | phosphocholine | | | | |
| Trimbow (Trinity | beclomethasone | Ethanol | pressurized inhalation, | adults: 2 puffs (30- | 320 | μg BID |
| Chiesi) | | anhydrous | solution | 160 μg) BID | | |
| Enerzair | mometasone | lactose | inhalation powder, hard | adults: 1 puff (70- | 220 | μg BID |
| Breezhaler | | monohydrate | capsules | 360 μg) once daily | | |
| (Novartis) | | | | | | |
| PTO27 | budesonide | | | adults: 1 puff (30- | 800 | μg BID |
| (AstraZeneca) | | | | 350) BID | | |
| FF/UMEC/VI | fluticasone furoate | | pressurized inhalation, | adults: 1 puff (40- | 400 μg once | |
| (GSK) | | | solution | 300 μg) once daily | daily | |
| B/F/G (Trinity | beclomethasone | | pressurized inhalation, | adults: 1 puff (30 - | 320 | μg BID |
| Chiesi) | | | solution | 250 μg)once daily | | |

### Example 17.—Treating PASC Pulmonary Patients with Modified Formulations

Following the methods disclosed herein, patients can be treated for PASC pulmonary sequalae using formulations with modified drug particulate size, solvent, drug formulation distribution, dispersal rates, and duration of dispersal. Suitable examples include inhaled corticosteroids, inhaled coformulations of corticosteroids and LABAs, and inhaled coformulations of corticosteroids, LABAs, and an antimuscarinic agent (Table 6).

TABLE 6
Dosing instructions for treating PASC pulmonary patients with inhaled
corticosteroid formulations comprising extrafine particles.

| Name and | Steroid | | Method of | | | |
|---|---|---|---|---|---|---|
| Manufacturer | component | Diluent | delivery | Delivered dosing | Steroid Daily Max | |
| Qvar RediHaler | beclomethasone | HFA- | pressurized, | adults: 1 or 2 puffs | 320 | μg BID |
| (Teva) | dipropionate | 134a, Ethanol, | metered-dose | (40-80 μg BID | | |
| | | Dehydrated | aerosol | | | |
| Qvar Autohaler | beclomethasone | HFA- | pressurized, | adults: 1 or 2 puffs | 320 | μg BID |
| (Teva) | dipropionate | 134a, Ethanol | metered-dose | (40-80 μg BID | | |
| | | | aerosol | | | |
| Flovent HFA | fluticasone | HFA-134a | pressurized, | adults: 2 puffs (88 | 1320 | μg BID |
| (GSK) | propionate | | metered-dose | μg) BID | | |
| | | | aerosol | | | |
| Flixotide Evohaler | fluticasone | HFA-134a ( | pressurized, | adults: 2 puffs (100 | 1320 | μg BID |
| (GSK) | propionate | | metered-dose | μg) BID | | |
| | | | aerosol | | | |
| Flovent Diskus | fluticasone | lactose | dry powder | adults: 2 puffs (100 | 1320 | μg BID |
| (GSK) | propionate | monohydrate | inhaler | μg) BID | | |
| Flixotide Diskus | fluticasone | lactose | dry powder | adults: 2 puffs (100- | 1320 | μg BID |
| (GSK) | propionate | monohydrate | inhaler | 250 mg) BID | | |
| Flovent Rotadisc | fluticasone | lactose | dry powder | adults: 1 puff (100 | 1320 | μg BID |
| (GSK) | propionate | monohydrate | inhaler | μg) BID | | |
| Arnuity Ellipta | fluticasone | lactose | dry powder | adults: 1 puff (100 | 400 μg once daily | |
| (GSK) | furoate | monohydrate | inhaler | μg) once daily | | |
| ArmonAir | fluticasone | lactose | dry powder | adults: 2 puffs (55 | 1320 | μg BID |
| Digihaler (Teva) | propionate | monohydrate | inhaler | μg) BID | | |
| Pulmicort | budesonide | lactose | dry powder | adults: 2 puffs (180 | 800 | μg BID |
| Flexhaler | | monohydrate | inhaler | μg) BID | | |
| (AstraZeneca) | | | | | | |
| Pulmicort | budesonide | / | dry powder | adults: 1 puff (200 | 800 | μg BID |
| Turbohaler | | | inhaler | μg) BID | | |
| (AstraZeneca) | | | | | | |
| Alvesco (Covis) | ciclesonide | HFA- | pressurized, | adults: 1 or 2 puffs | 320 | μg BID |
| | | 134a, Ethanol | metered-dose | (80-160 μg) BID | | |
| | | | aerosol | | | |
| Asmanex | mometasone | lactose | dry powder | adults: 2 puffs (100- | 220 | μg BID |
| Twisthaler | | monohydrate | inhaler | 200 μg) BID | | |
| (Organon Global) | | | | | | |
| Asmanex HFA | mometasone | HFA-227 | pressurized, | adults: 2 puffs (100- | 220 | μg BID |
| (Organon Global) | | | metered-dose | 200 μg) BID | | |
| | | | aerosol | | | |
| Symbicort | budesonide | lactose | DPI (dry powder | adults: 1-2 puffs | 800 | μg BID |
| (AstraZeneca) | | monohydrate | inhaled), MDI | (12-315 μg) BID | | |
| | | | with spacer, | | | |
| | | | nebulizer | | | |
| Duoresp Spiromax | budesonide | lactose | dry powder inhaler | adults: 1-2 puffs | 800 | μg BID |
| (Teva) | | monohydrate | | (12-315 μg) BID | | |
| Atectura | mometasone | lactose | Inhalation powder, | adults: 1 puff (24- | 220 | μg BID |
| Breezhaler | | monohydrate | hard capsule | 324 μg) once daily | | |
| (Novartis) | | | (inhalation powder). | | | |
| FOSTAIR | beclomethasone | Norflurane (HFA- | inhalation powder | adults: 1-2 puffs | 169.2 | μg BID |
| NEXThaler | dipropionate | 134a) | | (12-144 μg) BID | | |
| (Trinity Chiesi) | | | | | | |
| Advair Diskus | fluticasone | lactose | Inhalation powder | adults: 1-2 puffs | 1320 | μg BID |
| (GSK) | propionate | monohydrate | | (16-270 μg) BID | | |
| Advair HFA | fluticasone | 1,1,1,2- | pressurized | adults: 2 puffs (16- | 230 | μg BID |
| (GSK) | | tetrafluoroethane | inhalation, | 270 μg) BID | | |
| | | (HFA-134a) | suspension | | | |
| Airduo Respiclick | fluticasone | lactose | Inhalation powder | adults: 1 puff (4- | 1320 | μg BID |
| (Teva) | | monohydrate | | 67.5 μg) BID | | |
| Airduo Digihaler | fluticasone | lactose | Inhalation powder | adults: 1 puff (4- | 1320 | μg BID |
| (Teva) | | monohydrate | | 67.5 μg) BID | | |
| Wixela Inhub | fluticasone | lactose | Inhalation powder | | 1320 | μg BID |
| (Mylan) | | monohydrate | | | | |
| Seretide diskus | fluticasone | lactose | Inhalation powder | adults: 1 puff BID | 1320 | μg BID |
| (GSK) | | monohydrate | | | | |
| Aerivio ® | fluticasone | lactose | Inhalation powder | adults: 1 puff BID | 1320 | μg BID |
| Spiromax (Teva) | | monohydrate | | | | |
| Sirdupla (Mylan) | fluticasone | HFA 134a | pressurized | adults: 2 puffs BID | 1320 | μg BID |
| | | | inhalation, | | | |
| | | | suspension. | | | |
| Airflusal Forspiro | fluticasone | lactose | Inhalation powder | adults: 1 puff BID | 1320 | μg BID |
| (Areopharm) | | monohydrate | | | | |
| Breo Ellipta | fluticasone | lactose | Inhalation powder | adults: 1 puff (16- | 1320 | μg BID |
| (GSK) | | monohydrate | | 270 μg) once daily | | |
| Relvar Ellipta | fluticasone | lactose | Inhalation powder | adults: 1 puff (16 - | 1320 | μg BID |
| (GSK) | | monohydrate | | 324 μg) once daily | | |
| Dulera (Merck) | mometasone | anhydrous | pressurized, | adults: 2 puffs (28- | 220 | μg BID |
| | | alcohol | metered-dose | 324 μg) BID | | |
| | | | aerosol | | | |
| Trelegy Ellipta | fluticasone | lactose | | adults: 1-2 puffs | 1320 | μg BID |
| (GSK) | | monohydrate | | (16-270 μg) BID | | |
| Breztri aerosphere | budesonide | HFA 134a | pressurized, | adults: 1-2 puffs | 800 | μg BID |
| (AstraZeneca) | | | metered-dose | (12-315 μg) BID | | |
| | | | aerosol | | | |
| Trixeo Aerosphere | budesonide | Norflurane 1,2- | pressurized, | | 800 | μg BID |
| (AstraZeneca) | | distearoyl-sn- | metered-dose | | | |
| | | glycero-3- | aerosol | | | |
| | | phosphocholine | | | | |
| Trimbow (Trinity | beclomethasone | Ethanol | pressurized | adults: 2 puffs (12- | 320 | μg BID |
| Chiesi) | | anhydrous | inhalation, | 144 μg) BID | | |
| | | | solution | | | |
| Enerzair | mometasone | lactose | inhalation | adults: 1 puff(28- | 220 | μg BID |
| Breezhaler | | monohydrate | powder, hard | 324 μg) once daily | | |
| (Novartis) | | | capsules | | | |
| PTO27 | budesonide | | | adults: 1 puff (40- | 3200 | μg |
| (AstraZeneca) | | | | 225 μg) BID | | |
| FF/UMEC/VI | fluticasone furoate | | pressurized | adults: 1-2 puffs | 400 μg once daily | |
| (GSK) | | | inhalation, | (16-270 μg) BID | | |
| | | | solution | | | |
| B/F/G (Trinity | beclomethasone | | pressurized | adults: 1 puff (12- | 320 | μg BID |
| Chiesi) | | | inhalation, | 225 μg) once daily | | |
| | | | solution | | | |

### Example 18

Post-COVID cough was a facet that characterized many of these patients and often contributed to their medical condition and symptoms. This could often be managed as described in the aforementioned examples, alone or in various combinations with the other therapies previously described. Additionally, these patients' cough could be well managed with cough mixtures or tablets such as promethazine, codeine, and ephedrine (e.g., 12.5, 25 or 50 mg doses, or 25 or 50 mg tablets) twice a day or PRN, codeine, or dextromethorphan, or hydrocodone/pseudoephedrine/chlorpheniramine (Zutripro or Hydron PSC liquid 5 ml q4-6 5 ml), or intranasal therapies such as Avamys (fluticasone 27.5 μg spray or intranasal suspension), or as previously described, alone or with the inhaled or oral therapies described previously. Patients with cough were treated with these or cough suppressants such as codeine and/or dextromethorphan-based medications alone or in combination with inhaled therapies. It was observed that in these patients, where cough was a prominent part of their post-COVID sequelae, they both subjectively and objectively responded better to these therapies than to the therapies above alone. In certain embodiments, the method further comprises administering a cough elixir.

All references to patents, patent documents, articles, and internet citations are incorporated by references in their entireties for all purposes.

What is claimed is:

1. A method of treating a pulmonary disease which is the consequence of previous exposure of a subject to a severe acute respiratory syndrome coronavirus-2 (SARS-CoV-2), the method comprising administering to the subject of an effective amount of one or more corticosteroids.

2. The method of claim 1, wherein the one or more corticosteroids are chosen from hydrocortisone, methylprednisolone, budesonide, beclomethasone, fluticasone, fluticasone furoate, fluticasone propionate, mometasone, mometasone furoate, and vamorolone.

3. The method of claim 2, wherein the one or more corticosteroids is budesonide.

4. The method of claim 2, wherein the one or more corticosteroids is administered twice daily.

5. The method of claim 1, wherein the one or more corticosteroids are administered immediately after exposure to SARS-CoV-2 before initial recovery.

6. The method of claim 1, wherein the one or more corticosteroids are administered after initial recovery.

7. The method of claim 1, wherein the one or more corticosteroids are administered continuously for a period of time.

8. The method of claim 1, wherein the one or more corticosteroids are administered when the subject expresses a symptom which is the consequence of the previous exposure of the SARS-CoV-2 after initial recovery or before the subject partakes in an activity that may precipitate the symptom.

9. The method of claim 1 further comprising administering one or more leukotriene antagonists.

10. The method of claim 9, wherein the one or more leukotriene antagonists are chosen from zafirlukast, montelukast, and zileuton.

11. The method of claim 1 further comprising administering one or more beta-agonists.

12. The method of claim 1 further comprising administering one or more methylxanthines.

13. The method of claim 12, wherein the methylxanthine is chosen from theophylline, dyphylline, and aminophylline.

14. The method of claim 1 further comprising administering one or more bronchodilators.

15. The method of claim 14, wherein the one or more bronchodilators comprises an anticholinergic.

16. The method of claim 10, wherein the one or more bronchodilators comprises an antimuscarinic.

17. The method of claim 16, wherein the antimuscarinic is chosen from tiotropium, ipratropium, aclidinium, glycopyrronium, and/or salts thereof.

18. The method of claim 14, wherein the one or more bronchodilators comprises a beta-2 agonist.

19. The method of claim 18, wherein the beta-2 agonist is chosen from salbutamol, albuterol, salmeterol, and formoterol.

20. The method of claim 1, wherein the exposure of the subject to the SARS-CoV-2 virus is confirmed by a positive titer for the SARS-CoV-2, by a positive titer for antibodies to the SARS-CoV-2, or by one or more recognized clinical symptoms associated with infection by SARS-CoV-2.

21. The method of claim 20, wherein one or more recognized clinical symptoms are chosen from cough, shortness of breath, difficulty breathing, asthma, fever, chills, repeated shaking, muscle pain, headache, sore throat, loss of taste, loss of smell, blood clotting, stroke, pernio, and chilblain.

22. A method of reducing, delaying the onset of, or preventing symptoms which are the consequence of previous exposure of a subject to a severe acute respiratory syndrome coronavirus-2 (SARS-CoV-2), the method comprising administering to the subject of an effective amount of one or more corticosteroids.

23. The method of claim 22, wherein the one or more corticosteroids are chosen from hydrocortisone, methylprednisolone, budesonide, beclomethasone, fluticasone, fluticasone furoate, fluticasone propionate, mometasone, mometasone furoate, and vamorolone.

24. The method of claim 23, wherein the one or more corticosteroids is budesonide.

25. The method of claim 23, wherein the one or more corticosteroids is administered twice daily.

26. The method of claim 22, wherein the symptoms are chosen from cough, shortness of breath, difficulty breathing, asthma, fever, chills, repeated shaking, muscle pain, headache, sore throat, loss of taste, loss of smell, bloodclotting, stroke, pernio, and chilblain.

27. The method of claim 22, wherein the one or more corticosteroids are administered immediately after exposure to SARS-CoV-2 before initial recovery.

28. The method of claim 22, wherein the one or more corticosteroids are administered after initial recovery.

29. The method of claim 22, wherein the one or more corticosteroids are administered continuously for a period of time.

30. The method of claim 22, wherein the one or more corticosteroids are administered when the subject expresses a symptom which is the consequence of the previous exposure of the SARS-CoV-2 after initial recovery or before the subject partakes in an activity that may precipitate the symptom.
